SEQUENCE LISTING (55 sequences)

SEQ ID NO: 1 - DNA, 20 bp, Artificial Sequence
  TALE 28 Left DNA binding target
tgctgctatgcctcatcttc

SEQ ID NO: 2 - DNA, 16 bp, Artificial Sequence
  TALE 28 Right DNA binding target
ctggactatcaaggta

SEQ ID NO: 3 - DNA, 19 bp, Artificial Sequence
  TALE 33 Left DNA binding target
tcctgctgctatgcctcat

SEQ ID NO: 4 - DNA, 19 bp, Artificial Sequence
  TALE 33 Right DNA binding target
cttctggactatcaaggta

SEQ ID NO: 5 - DNA, 48 bp, Artificial Sequence
  TALE 28 DNA binding target
tgctgctatgcctcatcttcttgttggttcttctggactatcaaggta

SEQ ID NO: 6 - DNA, 51 bp, Artificial Sequence
  TALE 33 DNA binding target
tcctgctgctatgcctcatcttcttattggttcttctggactatcaaggta

SEQ ID NO: 7 - protein, 627 aa, Artificial Sequence
  TALE 28 Left DNA binding sequence
LTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGG

SEQ ID NO: 8 - protein, 491 aa, Artificial Sequence
  TALE 28 Right DNA binding sequence
LTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGG

SEQ ID NO: 9 - protein, 593 aa, Artificial Sequence
  TALE 33 Left DNA binding sequence
LTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGG

SEQ ID NO: 10 - protein, 593 aa, Artificial Sequence
  TALE 33 Right DNA binding sequence
LTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGG

SEQ ID NO: 11 - protein, 198 aa, Artificial Sequence
  FokI Nuclease
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 12 - RNA, 129 nt, Artificial Sequence
  TEV 5′UTR
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauag

SEQ ID NO: 13 - RNA, 28 nt, Artificial Sequence
  AT1G58420 5′UTR
auuauuacaucaaaacaaaaagccgcca

SEQ ID NO: 14 - RNA, 245 nt, Artificial Sequence
  ARC5-2 5′UTR
cuuaagggggcgcugccuacggagguggcagccaucuccuucucggcaucaagcuuaccauggugccccaggcccugcucuuggucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuac

SEQ ID NO: 15 - RNA, 177 nt, Artificial Sequence
  HCV 5′UTR
ugagugucguacagccuccaggccccccccucccgggagagccauaguggucugcggaaccggugaguacaccggaauugccgggaagacuggguccuuucuuggauaaacccacucuaugcccggccauuugggcgugcccccgcaagacugcuagccgaguaguguuggguugcg

SEQ ID NO: 16 - RNA, 89 nt, Artificial Sequence
  Human Albumin 5′UTR
aauuauugguuaaagaaguauauuagugcuaauuucccuccguuuguccuagcuuuucucuucugucaaccccacacgccuuuggcaca

SEQ ID NO: 17 - RNA, 569 nt, Artificial Sequence
  EMCV 5′UTR
cucccuccccccccccuaacguuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacguguguuuagucgagguuaaaaaacgucuaggccccccgaaccacggggacgugguuuuccuuugaaaaacacgaugauaau

SEQ ID NO: 18 - RNA, 158 nt, Artificial Sequence
  XBG 3′UTR
cuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacau

SEQ ID NO: 19 - RNA, 166 nt, Artificial Sequence
  Human haptoglobin 3′UTR
ugcaaggcuggccggaagcccuugccugaaagcaagauuucagccuggaagagggcaaaguggacgggaguggacaggaguggaugcgauaagaugugguuugaagcugaugggugccagcccugcauugcugagucaaucaauaaagagcuuucuuuugacccau

SEQ ID NO: 20 - RNA, 143 nt, Artificial Sequence
  Human Apolipoprotein E 3′UTR
acgccgaagccugcagccaugcgaccccacgccaccccgugccuccugccuccgcgcagccugcagcgggagacccuguccccgccccagccguccuccugggguggacccuaguuuaauaaagauucaccaaguuucacgca

SEQ ID NO: 21 - RNA, 220 nt, Artificial Sequence
  HCV 3′UTR
uagagcggcaaacccuagcuacacuccauagcuaguuucuuuuuuuuuuguuuuuuuuuuuuuuuuuuuuuuuuuuuuuuuuuuuuuuuccuuucuuuuccuucuuuuuuuccucuuuucuugguggcuccaucuuagcccuagucacggcuagcugugaaagguccgugagccgcaugacugcagagagugccguaacuggucucucugcagaucaugu

SEQ ID NO: 22 - RNA, 170 nt, Artificial Sequence
  Mouse albumin 3′UTR
acacaucacaaccacaaccuucucaggcuacccugagaaaaaaagacaugaagacucaggacucaucuuuucuguugguguaaaaucaacacccuaaggaacacaaauuucuuuaaacauuugacuucuugucucugugcugcaauuaauaaaaaauggaaagaaucuac

SEQ ID NO: 23 - RNA, 110 nt, Artificial Sequence
  Human alpha globin 3′UTR
gcuggagccucgguagccguuccuccugcccgcugggccucccaacgggcccuccuccccuccuugcaccggcccuuccuggucuuugaauaaagucugagugggcagca

SEQ ID NO: 24 - RNA, 123 nt, Artificial Sequence
  EMCV 3′UTR
uagugcagucacuggcacaacgcguugcccgguaagccaaucggguauacacggucgucauacugcagacaggguucuucuacuuugcaagauagucuagaguaguaaaauaaauaguauaag

SEQ ID NO: 25 - protein, 1073 aa, Artificial Sequence
  TALEN 28 Left protein
MDYKDHDGDYKDHDIDYKDDDDKGGSGPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 26 - protein, 937 aa, Artificial Sequence
  TALEN 28 Right protein
MDYKDHDGDYKDHDIDYKDDDDKGGSGPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 27 - protein, 1039 aa, Artificial Sequence
  TALEN 33 Left protein
MDYKDHDGDYKDHDIDYKDDDDKGGSGPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 28 - protein, 1039 aa, Artificial Sequence
  TALEN 33 Right protein
MDYKDHDGDYKDHDIDYKDDDDKGGSGPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 29 - DNA, 20 bp, Artificial Sequence
  Forward primer
cctaggaccccttctcgtgt

SEQ ID NO: 30 - DNA, 20 bp, Artificial Sequence
  Reverse primer
actgagccaggagaaacggg

SEQ ID NO: 31 - protein, 7 aa, Artificial Sequence
  SV40 Large T-Antigen NLS
PKKKRKV

SEQ ID NO: 32 - protein, 16 aa, Artificial Sequence
  nucleoplasmin NLS
KRPAATKKAGQAKKKK

SEQ ID NO: 33 - protein, 25 aa, Artificial Sequence
  EGL-13 NLS
MSRRRKANPTKLSENAKKLAKEVEN

SEQ ID NO: 34 - protein, 9 aa, Artificial Sequence
  c-Myc proto-oncoprotein NLS
PAAKRVKLD

SEQ ID NO: 35 - protein, 9 aa, Artificial Sequence
  TUS-protein NLS
KLKIKRPVK

SEQ ID NO: 36 - protein, 8 aa, Artificial Sequence
  FLAG tag
DYKDDDDK

SEQ ID NO: 37 - protein, 8 aa, Artificial Sequence
  FLAG tag
EYKEEEEK

SEQ ID NO: 38 - protein, 6 aa, Artificial Sequence
  hexa-histidine
HHHHHH

SEQ ID NO: 39 - protein, 10 aa, Artificial Sequence
  myc tag
EQKLISEEDL

SEQ ID NO: 40 - protein, 9 aa, Artificial Sequence
  influenza HA tag
YPYDVPDYA

SEQ ID NO: 41 - protein, 9 aa, Artificial Sequence
  AcV5 tag
SWKDASGWS

SEQ ID NO: 42 - protein, 13 aa, Artificial Sequence
  ALFA-tag
SRLEEELRRRLTE

SEQ ID NO: 43 - protein, 15 aa, Artificial Sequence
  AviTag
GLNDIFEAQKIEWHE

SEQ ID NO: 44 - protein, 13 aa, Artificial Sequence
  E-tag
GAPVPYPDPLEPR

SEQ ID NO: 45 - protein, 15 aa, Artificial Sequence
  S-tag
KETAAAKFERQHMDS

SEQ ID NO: 46 - protein, 8 aa, Artificial Sequence
  Strep-tag
WSHPQFEK

SEQ ID NO: 47 - protein, 11 aa, Artificial Sequence
  T7 tag
MASMTGGQQMG

SEQ ID NO: 48 - protein, 10 aa, Artificial Sequence
  Ty1 tag
EVHTNQDPLD

SEQ ID NO: 49 - protein, 14 aa, Artificial Sequence
  V5 tag
GKPIPNPLLGLDST

SEQ ID NO: 50 - protein, 11 aa, Artificial Sequence
  VSV-G tag
YTDIEMNRLGK

SEQ ID NO: 51 - protein, 8 aa, Artificial Sequence
  Xpress tag
DLYDDDDK

SEQ ID NO: 52 - protein, 763 aa, Artificial Sequence
  TALE 28 Left DNA binding sequence with N-terminal
VDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGG

SEQ ID NO: 53 - protein, 627 aa, Artificial Sequence
  TALE 28 Right DNA binding sequence with N-terminal
VDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGG

SEQ ID NO: 54 - protein, 729 aa, Artificial Sequence
  TALE 33 Left DNA binding sequence with N-terminal
VDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGG

SEQ ID NO: 55 - protein, 729 aa, Artificial Sequence
  TALE 33 Right DNA binding sequence with N-terminal
VDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGG

(19) United States
(12) Patent Application Publication
(10) Pub. No.: US 2024/0316090 A1
(43) Pub. Date: Sep. 26, 2024

(54) TRANSCRIPTION ACTIVATOR-LIKE EFFECTOR NUCLEASES (TALENS) TARGETING HBV

(71) Applicants: ARCTURUS THERAPEUTICS, INC., San Diego, CA (US); JANSSEN PHARMACEUTICALS, INC., Titusville, NJ (US)

(72) Inventors: Ramón Díaz TRELLES, San Diego, CA (US); Man Lu LAM, Titusville, NJ (US); Kiyoshi TACHIKAWA, San Diego, CA (US); Priya Prakash KARMALI, San Diego, CA (US); Padmanabh CHIVUKULA, San Diego, CA (US)

(21) Appl. No.: 18/259,535

(22) PCT Filed: Dec. 10, 2021
(86) PCT No.: PCT/US2021/062749

§ 371 Date: Jun. 27, 2023

Related U.S. Application Data
(60) Provisional application No. 63/131,145, filed on Dec. 28, 2020.

(51) Int. Cl. A61K 31/7105; A61K 45/06; A61P 31/20; C12N 9/22; C12N 15/90

(52) U.S. Cl. CPC A61K 31/7105; A61K 45/06; A61P 31/20; C12N 9/22; C12N 15/907

(57) ABSTRACT

Nucleic acid molecules encoding transcription activator like effector nucleases (TALENs) for targeting a hepatitis B virus (HBV) genome are described. Also described are compositions, host cells, lipids, and pharmaceutical compositions containing the TALENs. Methods for treating hepatitis infection, particularly in individuals having chronic HBV infection, using the pharmaceutical compositions of the invention are also described.

### CROSS-REFERENCE TO RELATED APPLICATION

This application claims the benefit of U.S. Provisional Patent Application No. 63/131,145, filed Dec. 28, 2020, which is incorporated by reference herein in its entirety.

### FIELD OF THE INVENTION

The present disclosure relates generally to the field of molecular biology and genetic engineering, including the use of transcription activator-like effector nuclease (TALEN) sequences for gene targeting and regulating gene expression.

### REFERENCE TO SEQUENCE LISTING SUBMITTED ELECTRONICALLY

This application contains a sequence listing, which is submitted electronically via EFS-Web as an ASCII formatted sequence listing with a file name “065814_11515_Sequence_Listing_ST25” and a creation date of Dec. 7, 2021 and having a size of 91 kb. The sequence listing submitted via EFS-Web is part of the specification and is herein incorporated by reference in its entirety.

### BACKGROUND OF THE INVENTION

Hepatitis B virus (HBV) is a small 3.2-kb hepatotropic DNA virus that encodes four open reading frames and seven proteins. About two billion people are infected with HBV, and approximately 240 million people have chronic hepatitis B infection (chronic HBV), characterized by persistent virus and subvirus particles in the blood for more than 6 months (Cohen et al. J. Viral Hepat. (2011) 18(6), 377-83). Persistent HBV infection leads to T-cell exhaustion in circulating and intrahepatic HBV-specific CD4+ and CD8+ T-cells through chronic stimulation of HBV-specific T-cell receptors with viral peptides and circulating antigens. As a result, T-cell polyfunctionality is decreased (i.e., decreased levels of IL-2, tumor necrosis factor (TNF)-α, IFN-γ, and lack of proliferation).

Chronic HBV (CHB) is currently treated with IFN-α and nucleoside or nucleotide analogs, but there is no ultimate cure due to the persistence in infected hepatocytes of an intracellular viral replication intermediate called covalently closed circular DNA (cccDNA), which plays a fundamental role as a template for viral RNAs, and thus new virions. It is thought that induced virus-specific T-cell and B-cell responses can effectively eliminate cccDNA-carrying hepatocytes. Current therapies targeting the HBV polymerase suppress viremia, but offer limited effect on cccDNA that resides in the nucleus and related production of circulating antigen. The most rigorous form of a cure may be elimination of HBV cccDNA from the organism, which has neither been observed as a naturally occurring outcome nor as a result of any therapeutic intervention. However, loss of HBV surface antigens (HBsAg) is a clinically credible equivalent of a cure, since disease relapse can occur only in cases of severe immunosuppression, which can then be prevented by prophylactic treatment. Thus, at least from a clinical standpoint, loss of HBsAg is associated with the most stringent form of immune reconstitution against HBV.

Hepatitis D virus (HDV) infection only occurs in the context of co-infection with HBV as it requires the presence of HBsAg for HDV to form infectious virus particles. Co-infection is associated with earlier development of liver cirrhosis, increased risk for development of hepatocellular carcinoma (HCC) and increased liver-related and overall mortality.

It would therefore be useful to have compositions and methods that would enable targeted cleavage of the HBV genome in chronically infected patients, with high efficiency and reduced cytotoxicity.

### SUMMARY OF THE INVENTION

Accordingly, there is an unmet medical need in the treatment of hepatitis, particularly hepatitis B virus (HBV), and more particularly chronic HBV, for targeted gene editing of HBV cccDNA, specifically HBsAg. The invention satisfies this need by providing compositions and methods for inducing directed cleavage of specific DNA sequences.

In a general aspect, provided herein is a method for treating hepatitis infection in a subject in need thereof, comprising administering to the subject a combination of mRNA molecules comprising:
(1) a first mRNA molecule comprising a polynucleotide sequence encoding a first transcription activator like effector nuclease (TALEN) monomer comprising a first TALE DNA binding domain and a first nuclease catalytic domain, wherein the first TALE DNA binding domain is capable of binding to a first half-site sequence of a target nucleic acid sequence within an HBV genome; and
(2) a second mRNA molecule comprising a polynucleotide sequence encoding a second TALEN monomer comprising a second TALE DNA binding domain and a second nuclease catalytic domain, wherein the second TALE DNA binding domain is capable of binding to a second half-site sequence of the target nucleic acid sequence;
wherein the first TALEN monomer and the second TALEN monomer are capable of forming a dimer that cleaves the target nucleic acid sequence when the first TALE DNA binding domain binds to the first half-site sequence and the second TALE DNA binding domain binds to the second half-site sequence,
preferably, the first nuclease catalytic domain is a first FokI nuclease catalytic domain and the second nuclease catalytic domain is a second FokI nuclease catalytic domain.

In certain embodiments, the target nucleic acid sequence is within the sequence that encodes HBsAg and HBV polymerase (pol), preferably,
(a) the first half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 1, and the second half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 2; or
(b) the first half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 3, and the second half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 4; or
(c) the target nucleic acid sequence comprises the polynucleotide sequence at least 90% identical, such as at least 90%, 91%, 92%, 93%, 94%, 95%, 96%, 97%, 98%, 99% or 100% identical, to the nucleic acid sequence of SEQ ID NO: 5 or SEQ ID NO: 6.

In certain embodiments, the first and second mRNA molecules each further comprise one or more, preferably all, of a 5′ cap, a 5′-UTR, a sequence encoding a nuclear localization signal, a sequence encoding an N-terminal domain, a sequence encoding a C-terminal domain, a 3′-UTR, and a poly adenosine tail.

In certain embodiments, the first TALE DNA binding domain comprises an amino acid sequence at least 90% identical to SEQ ID NO: 7 or SEQ ID NO: 9, the second TALE DNA binding domain comprises an amino acid sequence at least 90% identical to SEQ ID NO: 8 or SEQ ID NO: 10, respectively, and the first and second FokI nuclease catalytic domain each comprise an amino acid sequence at least 90% identical to SEQ ID NO: 11.

In certain embodiments, the HBV genome is of one or more of genotypes A, B, C, D, E, F, G, H, I and J.

In certain embodiments, the method further comprises administering to the subject a second therapeutic composition, preferably comprising an anti-HBV agent.

In certain embodiments, the subject has an HBV infection, preferably a chronic HBV infection.

In certain embodiments, the subject is co-infected with HBV and HDV.

In another general aspect, provided herein is a composition comprising a combination of mRNA molecules encapsulated in lipid nanoparticles comprising:
(1) a first mRNA molecule comprising a polynucleotide sequence encoding a first transcription activator like effector nuclease (TALEN) monomer comprising a first TALE DNA binding domain and a first nuclease catalytic domain, wherein the first TALE DNA binding domain is capable of binding to a first half-site sequence of a target nucleic acid sequence within an HBV genome; and
(2) a second mRNA molecule comprising a polynucleotide sequence encoding a second TALEN monomer comprising a second TALE DNA binding domain and a second nuclease catalytic domain, wherein the second TALE DNA binding domain is capable of binding to a second half-site sequence of the target nucleic acid sequence;
wherein the first TALEN monomer and the second TALEN monomer are capable of forming a dimer that cleaves the target nucleic acid sequence when the first TALE DNA binding domain binds to the first half-site sequence and the second TALE DNA binding domain binds to the second half-site sequence,
preferably, the first nuclease catalytic domain is a first FokI nuclease catalytic domain and the second nuclease catalytic domain is a second FokI nuclease catalytic domain.

In certain embodiments, the target nucleic acid sequence is within the sequence that encodes HBsAg and HBV polymerase (pol), preferably,
(a) the first half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 1, and the second half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 2; or
(b) the first half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 3, and the second half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 4, or
(c) the target nucleic acid sequence comprises the polynucleotide sequence at least 90% identical, such as at least 90%, 91%, 92%, 93%, 94%, 95%, 96%, 97%, 98%, 99% or 100% identical, to the nucleic acid sequence of SEQ ID NO: 5 or SEQ ID NO: 6.

In certain embodiments, the first and second mRNA molecules each further comprise one or more, preferably all, of a 5′ cap, a 5′-UTR, a sequence encoding a nuclear localization signal, a sequence encoding an N-terminal domain, a sequence encoding a C-terminal domain, a 3′-UTR, and a poly adenosine tail.

In certain embodiments, the first TALE DNA binding domain comprises an amino acid sequence at least 90% identical to SEQ ID NO: 7 or SEQ ID NO: 9, the second TALE DNA binding domain comprises an amino acid sequence at least 90% identical to SEQ ID NO: 8 or SEQ ID NO: 10, respectively, and the first and second FokI nuclease catalytic domain each comprise an amino acid sequence at least 90% identical to SEQ ID NO: 11.

In certain embodiments, the HBV genome is of one or more of genotypes A, B, C, D, E, F, G, H, I and J.

In certain embodiments, the lipid nanoparticles encapsulating the combination of mRNA molecules comprise a cationic lipid and at least one other lipid selected from the group consisting of anionic lipids, zwitterionic lipids, neutral lipids, steroids, polymer conjugated lipids, phospholipids, glycolipids, and combinations thereof.

In another general aspect, provided herein is a combination of:
(1) a first nucleic acid, preferably a first mRNA molecule, comprising a polynucleotide sequence encoding a first transcription activator like effector nuclease (TALEN) monomer comprising a first TALE DNA binding domain and a first FokI nuclease catalytic domain, wherein the first TALE DNA binding domain is capable of binding to a first half-site sequence of a target nucleic acid sequence within an HBV genome, and the first half-site sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 1; and
(2) a second nucleic acid, preferably a second mRNA molecule, comprising a polynucleotide sequence encoding a second transcription activator like effector nuclease (TALEN) monomer comprising a second TALE DNA binding domain and a second FokI nuclease catalytic domain, wherein the second TALE DNA binding domain is capable of binding to a second half-site sequence of the target nucleic acid sequence, and the second half-site sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 2;
wherein the first TALEN monomer and the second TALEN monomer are capable of forming a dimer that cleaves the target nucleic acid sequence when the first TALE DNA binding domain binds to the first half-site sequence and the second TALE DNA binding domain binds to the second half-site sequence.

In certain embodiments, the HBV genome is of one or more of genotypes A, B, C, D, E, F, G, H, I and J.

In another general aspect, provided herein is a composition comprising a combination of a first nucleic acid and a second nucleic acid as described herein, wherein the first and second nucleic acids are separately or jointly encapsulated in lipid nanoparticles.

In another general aspect, provided herein is a nucleic acid molecule encoding at least one of a first mRNA molecule and a second mRNA molecule as described herein.

In another general aspect, provided herein is an isolated host cell comprising a nucleic acid molecule as described herein.

In particular embodiments, the host cell is a hepatocyte.

In another general aspect, provided herein is a pharmaceutical composition comprising a composition as described herein, a combination as described herein, a nucleic acid molecule as described herein, or an isolated host cell as described herein, and a pharmaceutically acceptable carrier.

In another general aspect, provided herein is a method of cleaving a target nucleic acid sequence in an HBV genome, comprising contacting the HBV genome with a composition as described herein, a combination as described herein, a nucleic acid molecule as described herein, an isolated host cell as described herein, or a pharmaceutical composition as described herein.

In another general aspect, provided herein is a method for inducing gene editing of a target nucleic acid sequence in an HBV genome, comprising contacting the HBV genome with a composition as described herein, a combination as described herein, a nucleic acid molecule as described herein, an isolated host cell as described herein, or a pharmaceutical composition as described herein.

In certain embodiments, the HBV genome is of one or more of genotypes A, B, C, D, E, F, G, H, I and J.

In another general aspect, provided herein is a method for treating a hepatitis infection in a subject in need thereof, comprising administering to the subject a pharmaceutical composition as described herein.

In another general aspect, provided herein is a method for reducing infection and/or replication of HBV in a subject, comprising administering to the subject the pharmaceutical composition as described herein.

In certain embodiments, the method further comprises administering to the subject a second therapeutic composition, preferably comprising an anti-HBV agent.

In certain embodiments, the subject has an HBV infection, preferably a chronic HBV infection.

In certain embodiments, the subject is co-infected with HBV and HDV.

In certain embodiments, an expression level of one or more of HBsAg, HBeAg, HBV DNA, HBV cccDNA, or integrated HBV DNA is reduced in the subject. In particular embodiments, the expression level is a hepatocyte level, a nuclear or cellular level, a liver level, a serum level, or a plasma level.

In another general aspect, provided herein is a method of producing a TALEN comprising transcribing the nucleic acid molecule as described herein, in vitro or in vivo.

In another general aspect, provided herein is a pharmaceutical composition as described herein for use in treating a hepatitis B virus (HBV)-induced disease in a subject in need thereof, preferably wherein the subject has chronic HBV infection.

In certain embodiments, the HBV-induced disease is selected from the group consisting of advanced fibrosis, cirrhosis and hepatocellular carcinoma (HCC), optionally in combination with another therapeutic agent, preferably another anti-HBV agent.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

The patent or application file contains at least one drawing executed in color. Copies of this patent or patent application publication with color drawing(s) will be provided by the Office upon request and payment of the necessary fee.

FIG. 1A-FIG. 1C show TALEN 28 and 33 target sites within HBV DNA genome. FIG. 1A: Schematic representation of the HBV genome and the overlapping open reading frames of the viral proteins (preC/Core, pol, preS1, preS2, HBsAg, and HBx). TALEN 28 and 33 target a site within the gene that encodes HBsAg/pol. FIG. 1B: HBV DNA sequences targeted by the left and right TALEN arms of TALEN28 (top, SEQ ID NO: 5) and TALEN33 (bottom, SEQ ID NO: 6). FIG. 1C: Schematic representation of the TALEN mRNA construct. The HBV DNA binding domain is located within an array of 15-19 TALE repeats, each of them containing two amino acids known as Repeat Variable Diresidues (RVDs) designed to target the HBV sequence.

FIG. 2A-FIG. 2B show HBV TALEN 28 target sequence conservation analysis. Conservation logos were generated based on the abundancy of each targeted nucleotide for TALEN 28 left (FIG. 2A) and right (FIG. 2B) arms. The higher the conservation, the larger the size of the abundant nucleotide(s).

FIG. 3A-FIG. 3B show HBV TALEN 33 target sequence conservation analysis. Conservation logos generated based on abundancy of each targeted nucleotide for TALEN 33 left (FIG. 3A) and right (FIG. 3B) arms. The higher the conservation, the larger the size of the abundant nucleotide(s).

FIG. 4A-FIG. 4C show that TALEN 28 and TALEN 33 target broad HBV genotype coverage. The left TALEN of each of TALEN 28 (FIG. 4A) and TALEN 33 (FIG. 4B) targets a highly conserved sequence, with a single mismatch within the right TALEN arm observed in genotype (GT) A and GT C. FIG. 4C shows DNA cleavage results of in vitro translated TALEN proteins against HBV DNA isolates from GTs A through H.

FIG. 5A-FIG. 5D show the effects of TALEN 28 and TALEN 33 in HepG2.2.15 cells. FIG. 5A: TALEN plasmids were transcribed in vitro and transfected into cells. 24 hours after transfection, nuclei were stained with DAPI, and TALEN expression in nuclei was confirmed by immunofluorescence. 6 days later, cells were assessed for the effects of TALENs activity on HBsAg secreted protein levels in the cell culture media (FIG. 5B), cell viability (FIG. 5C) and HBV DNA gene editing (FIG. 5D).

FIG. 6A-FIG. 6E show the effect of TALEN 28 in HBV infected primary human hepatocytes (PHH). FIG. 6A: Schematic illustration of HBV infection and TALEN mRNA transfection using PHHs. PHHs were treated with or without TALEN 28 mRNA pairs on day 5 post HBV infection. FIG. 6B: Expression of TALEN 28 was detected by immunofluorescence (red) and cell nuclei were stained by using DAPI (blue) at 6 hours post transfection. FIG. 6C: Antiviral effect against HBsAg and HBeAg production from TALEN 28 treated cells. Levels of HBsAg and HBeAg were determined on day 11 post infection. FIG. 6D: TALEN-induced targeted cccDNA sequence disruptions were examined by T7E1 digestion assays 6 days after TALENs treatment. Asterisks indicate disrupted sequences. FIG. 6E: Gene edited cccDNA showed mostly deletions within the TALEN 28 target sequence. Sequence was analyzed by next generation sequencing.

FIG. 7A-FIG. 7D show the effect of TALEN 33 in HBV infected PHHs. FIG. 7A: Schematic illustration of HBV infection and TALEN mRNA transfection using PHHs. PHHs were treated with or without TALEN 33 mRNA pairs on day 5 post HBV infection. FIG. 7B: Expression of TALEN 33 was detected by immunofluorescence (green) and cell nuclei were stained by using DAPI (blue) at 6 hours post transfection. FIG. 7C: Antiviral effect against HBsAg and HBeAg production from TALEN 33 treated cells. Levels of HBsAg and HBeAg in the presence or absence of TALEN 33 were determined on day 11 post infection. Data were calculated from at least 3 independent studies and represent average values with error bars. FIG. 7D: TALEN-induced targeted cccDNA sequence disruptions were examined by T7E1 digestion assays. Asterisks indicate disrupted sequences.

FIG. 8 shows that TALEN 33 edited cccDNA from PHHs infected with HBV GT A to D. A T7E1 assay was performed on cccDNA extracted from HBV infected PHHs, either treated (+) or not treated (−) with TALEN 33 encoding mRNA pairs. Asterisks indicate disrupted sequences.

FIG. 9A-FIG. 9C show that TALEN 33 mRNA pairs encapsulated into lipid nanoparticles (LNP-HBV TALENS) reduced serum HBsAg (FIG. 9A) and serum HBV DNA (FIG. 9B), and showed dose-dependent gene editing activity (FIG. 9C) in AAV-HBV infected mice.

FIG. 10A-FIG. 10B shows a comparison of LNP-HBV TALEN to the HBV treatment entecavir (ETV) in AAV-HBV infected mice. Sustained reduction of plasma HBsAg (FIG. 10A) and HBV DNA (FIG. 10B) over 91 days was observed with a single dose of LNP-HBV TALEN, but not the non-HBV targeting TTR TALEN. ETV administered QD for 7 days reduced plasma HBV DNA, but viral rebound was observed following treatment cessation.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications, and publications cited herein are incorporated by reference as if set forth fully herein.

It must be noted that as used herein and in the appended claims, the singular forms “a,” “an,” and “the” include plural reference unless the context clearly dictates otherwise.

Unless otherwise stated, any numerical value, such as a % sequence identity or a % sequence identity range described herein, are to be understood as being modified in all instances by the term “about.” Thus, a numerical value typically includes ±10% of the recited value. For example, a dosage of 10 mg includes 9 mg to 11 mg. As used herein, the use of a numerical range expressly includes all possible subranges, all individual numerical values within that range, including integers within such ranges and fractions of the values unless the context clearly indicates otherwise.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise,” and variations such as “comprises” and “comprising,” will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the invention can be replaced with the term “consisting of” or “consisting essentially of” to vary scopes of the disclosure.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. The term “complementary nucleotide bases” means a pair of nucleotide bases that form hydrogen bonds with each other. Adenine (A) pairs with thymine (T) or with uracil (U) in RNA, and guanine (G) pairs with cytosine (C). Complementary segments or strands of nucleic acid that hybridize (i.e. join by hydrogen bonding) with each other. By “complementary” is meant that a nucleic acid can form hydrogen bond(s) with another nucleic acid sequence either by traditional Watson-Crick or by other non-traditional modes of binding. Nucleic acid molecules can have any three-dimensional structure. A nucleic acid molecule can be double-stranded or single-stranded (e.g., a sense strand or an antisense strand). Non-limiting examples of nucleic acid molecules include genes, gene fragments, exons, introns, messenger RNA (mRNA), transfer RNA, ribosomal RNA, siRNA, micro-RNA, tracrRNAs, crRNAs, guide RNAs, ribozymes, cDNA, recombinant polynucleotides, branched polynucleotides, nucleic acid probes and nucleic acid primers. A nucleic acid molecule may contain unconventional or modified nucleotides. The terms “polynucleotide sequence” and “nucleic acid sequence” as used herein interchangeably refer to the sequence of a polynucleotide molecule.

The phrases “percent (%) sequence identity” or “% identity” or “% identical to” when used with reference to an amino acid sequence describe the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 90%, 91%, 92%, 93%, 94%, 95%, 97%, 98%, 99%, or 100% identity over the full-length of the amino acid sequences) may be determined, when the sequences are compared and aligned for maximum correspondence as measured using a sequence comparison algorithm as known in the art, or when manually aligned and visually inspected. The same determination may be made for nucleotide sequences. The sequences which are compared to determine sequence identity may thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g using the NCBI BLAST algorithm (Altschul S F, et al (1997), Nucleic Acids Res. 25:3389-3402).

The term “binding” or “specific binding” as used herein refers to sequence-specific, non-covalent interactions between macromolecules (e.g., between a protein and a nucleic acid). Not all components of a binding interaction need be sequence-specific (e.g., contacts with phosphate residues in a DNA backbone), as long as the interaction as a whole is sequence-specific.

As used herein, the terms and phrases “combination,” “in combination,” “in combination with,” “co-delivery,” and “administered together with” in the context of the administration of two or more therapies or components to a subject refers to simultaneous administration of two or more therapies or components, such as two nucleic acid molecules, e.g., mRNA molecules, or a therapeutic composition and a lipid. “Simultaneous administration” can be administration of the two components at least within the same day. When two components are “administered together with” or “administered in combination with,” they can be administered in separate compositions sequentially within a short time period, such as 24, 20, 16, 12, 8 or 4 hours, or within 1 hour, or they can be administered in a single composition at the same time. The use of the term “in combination with” does not restrict the order in which therapies or components are administered to a subject. For example, a first therapy or component (e.g. first nucleic acid molecule) can be administered prior to (e.g., 5 minutes to one hour before), concomitantly with or simultaneously with, or subsequent to (e.g., 5 minutes to one hour after) the administration of a second therapy or component (e.g., second nucleic acid molecule). In some embodiments, a first therapy or component (e.g. first nucleic acid molecule) and a second therapy or component (e.g., second nucleic acid molecule) are administered in the same composition. In other embodiments, a first therapy or component (e.g. first nucleic acid molecule) and a second therapy or component (e.g., second nucleic acid molecule) are administered in separate compositions.

The term “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

The term “delivery” refers to the act or manner of delivering a compound, substance, entity, moiety, cargo or payload.

The term “delivery agent” refers to any substance which facilitates, at least in part, the in vivo delivery of a polynucleotide to targeted cells.

The term “engineered” refers to a molecule designed to have a feature or property, whether structural or chemical, that varies from a starting point, wild type or native molecule.

The term “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

The term “lipid” means an organic compound that comprises an ester of fatty acid and is characterized by being insoluble in water, but soluble in many organic solvents. Lipids are usually divided into at least three classes: (1) “simple lipids,” which include fats and oils as well as waxes; (2) “compound lipids,” which include phospholipids and glycolipids; and (3) “derived lipids” such as steroids.

The term “lipid delivery vehicle” means a lipid formulation that can be used to deliver a therapeutic nucleic acid (e.g., mRNA) to a target site of interest (e.g., cell, tissue, organ, and the like). The lipid delivery vehicle can be a nucleic acid-lipid particle, which can be formed from a cationic lipid, a non-cationic lipid (e.g., a phospholipid), a conjugated lipid that prevents aggregation of the particle (e.g., a PEG-lipid), and optionally cholesterol. Typically, the therapeutic nucleic acid (e.g., mRNA) may be encapsulated in the lipid portion of the particle, thereby protecting it from enzymatic degradation.

The term “lipid encapsulated” means a lipid particle that provides a therapeutic nucleic acid such as an mRNA with full encapsulation, partial encapsulation, or both. In a preferred embodiment, the nucleic acid (e.g., mRNA) is fully encapsulated in the lipid particle.

As used herein, a “non-naturally occurring” nucleic acid or polypeptide refers to a nucleic acid or polypeptide that does not occur in nature. A “non-naturally occurring” nucleic acid or polypeptide can be synthesized, treated, fabricated, and/or otherwise manipulated in a laboratory and/or manufacturing setting. In some cases, a non-naturally occurring nucleic acid or polypeptide can comprise a naturally occurring nucleic acid or polypeptide that is treated, processed, or manipulated to exhibit properties that were not present in the naturally occurring nucleic acid or polypeptide, prior to treatment. As used herein, a “non-naturally occurring” nucleic acid or polypeptide can be a nucleic acid or polypeptide isolated or separated from the natural source in which it was discovered, and it lacks covalent bonds to sequences with which it was associated in the natural source. A “non-naturally occurring” nucleic acid or polypeptide can be made recombinantly or via other methods, such as chemical synthesis.

As used herein, the term “operably linked” refer to a linkage or a juxtaposition wherein the components so described are in a relationship permitting them to function in their intended manner. For example, a regulatory sequence operably linked to a nucleic acid sequence of interest is capable of directing the transcription of the nucleic acid sequence of interest, or a signal sequence operably linked to an amino acid sequence of interest is capable of secreting or translocating the amino acid sequence of interest over a membrane.

As used herein, “subject” means any animal, preferably a mammal, most preferably a human, who will be or has been treated by a method according to an embodiment of the application. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more preferably a human. A human subject can include a patient. The compounds, compositions, and methods described herein can be useful in both human therapy and veterinary applications for species that can be chronically infected by HBV. In some embodiments, the subject has an HBV infection, more particularly a chronic HBV (CHB) infection. The subject can have a CHB, with or without viral co-infection. As used herein, a “viral co-infection” refers to an infection with at least two types of virus. A “viral co-infection” can be an infection with at least two types of virus simultaneously. A “viral co-infection” can also be a superinfection, wherein an infection with one or more types of virus is in addition to a pre-existing infection with one or more other types of virus. In some embodiment, the subject has an HBV-HDV co-infection. Preferably, the subject has a CHB-HDV co-infection. More preferably, the subject has a viral co-infection with CHB and chronic HDV.

A “target site” or “target sequence” is a nucleic acid sequence that defines a portion of a nucleic acid to which a binding molecule or domain will bind, provided sufficient conditions for binding exist. A “half-site sequence” of a target sequence as used herein refers to a portion of the target sequence to which a binding molecule or domain will bind, provided sufficient conditions for binding exist. There are generally two half-site sequences within a target nucleic acid sequence, which may be separated by a spacer sequence. For example, a first TALE DNA binding domain can bind to a first half-site sequence of a target nucleic acid and a second TALE DNA binding domain can bind to a second half-site sequence of the same target nucleic acid. Preferably, the first half-site sequence and the second half-site sequence are different and are separated by a spacer sequence.

The description herein is directed to various embodiments of the application. The discussion of any embodiment is meant only to be exemplary and is not intended to suggest that the scope of the disclosure, including the claims, is limited to these examples. For example, while embodiments of nucleic acid sequences of the application described herein may contain particular components, including, but not limited to, certain TALE DNA binding domains, nuclease catalytic domains, etc. arranged in a particular order, those having ordinary skill in the art will appreciate that the concepts disclosed herein may equally apply to other components arranged in other orders that can be used in nucleic acid sequences of the application. The application contemplates use of any of the applicable components in any combination having any sequence that can be used in nucleic acid sequences of the application, whether or not a particular combination is expressly described.

### Hepatitis

Hepatitis is an inflammation of the liver, most commonly caused by a viral infection. There are five main hepatitis viruses, referred to as types A, B, C, D and E. Hepatitis A and E are typically caused by ingestion of contaminated food or water. Hepatitis B, C and D usually occur as a result of parenteral contact with infected body fluids (e.g., from blood transfusions or invasive medical procedures using contaminated equipment). Hepatitis B is also transmitted by sexual contact.

Hepatitis A virus (HAV) is an enterically transmitted viral disease that causes fever, malaise, anorexia, nausea, abdominal discomfort and jaundice. HAV is normally acquired by fecal-oral route, by either person-to-person contact, ingestion of contaminated food or water or transmission by pooled plasma products. The absence of a lipid envelope makes HAV very resistant to physicochemical inactivation, and the virus can withstand conventional heat treatment of blood products. The development of sensitive and specific diagnostic assays to identify HAV antigens and/or antibodies in infected individuals as well as nucleic acid-based tests to detect viremic samples to exclude them from transfusion represents an important public health challenge.

Hepatitis A virus (HAV) is a small, nonenveloped, spherical virus classified in the genus Hepatovirus of the Picomaviridae family. The HAV genome consists of a single-strand, linear, 7.5 kb RNA molecule encoding a polyprotein precursor that is processed to yield the structural proteins and enzymatic activities required for viral replication. HAV encodes four capsid proteins (A, B, C and D) which contain the major antigenic domains recognized by antibodies of infected individuals. In addition to the capsid proteins, antigenic domains have been reported in nonstructural proteins such as 2A and the viral encoded protease.

Hepatitis B virus (HBV) infects humans and may result in two clinical outcomes. In the majority of clinical infections in adults (90-95%), the virus is cleared after several weeks or months, and the patient develops a lifelong immunity against re-infection. In the remaining cases, however, the virus is not eliminated from the tissues, and the patient remains chronically infected. The sequelae of chronic infection are serious: such individuals are highly likely to develop scarring of the liver tissue (cirrhosis) and may eventually develop hepatocellular carcinoma. HBV is transmitted via infected blood or other body fluids, especially saliva and semen, during delivery, sexual activity, or sharing of needles contaminated by infected blood.

Chronic hepatitis B (CHB) infection is the most common cause of liver cirrhosis and hepatocellular carcinoma (HCC), with an estimated 500,000-900,000 deaths per year. Continuing HBV replication increases the risk of progression to cirrhosis and HCC.

Hepatitis C virus (HCV) is the causal agent for a largely chronic liver infection originally identified as non-A, non-B hepatitis. HCV has infected about four million people in the United States and 170 million worldwide, about four times as many as HIV and accounts for 90 to 95% of the hepatitis attributable to blood transfusion. It is presumed that the primary route of infection is through contact with contaminated bodily fluids, especially blood, from infected individuals. HCV infection is one of the primary causes of liver transplantation in the United States and other countries. Approximately 40-50% of the liver transplants in the United States are based on HCV infections. The disease frequently progresses to chronic liver damage. While the pathology of HCV infection affects mainly the liver, the virus is found in other cell types in the body including peripheral blood lymphocytes.

HCV is an RNA virus of the Flaviviridae, genus Hepacivirus, and is most closely related to the pestiviruses, BVDV and GBV-B. The HCV genome is composed of a single positive strand of RNA, approximately 9.6 kb in length. The HCV genome possesses a continuous, translational open reading frame (ORF) that encodes a polyprotein of about 3,000 amino acids. The structural protein(s) appear to be encoded in approximately the first quarter of the N-terminus region of the ORF, the remainder coding for non-structural proteins. The polyprotein serves as the precursor to at least 10 separate viral proteins critical for replication and assembly of progeny viral particles. The organization of structural and non-structural proteins in the HCV polyprotein is as follows: C-E1-E2-p7-NS2-NS3-NS4a-NS4b-NS5α-NS5b.

The hepatitis delta virus (HDV) is a satellite RNA virus dependent on hepatitis B surface antigens to assemble its envelope and form new virions to propagate infection. HDV has a small 1.7 Kb genome making it the smallest known human virus. The hepatitis D circular genome is unique among animal viruses because of the high GC nucleotide content. However, HDV is the most severe form of viral hepatitis. Compared with other agents of viral hepatitis, acute HDV infection is more often associated with fulminant hepatitis, a rapidly progressive, often fatal form of the disease in which massive amounts of the liver are destroyed. Chronic type D hepatitis is typically characterized by necroinflammatory lesions, similar to chronic HBV infection, but is more severe, and frequently progresses rapidly to cirrhosis and liver failure, accounting for the disproportionate association of chronic HDV infection with terminal liver disease. Although HDV infection affects fewer individuals than HBV alone, the resulting acute or chronic liver failure is a common indication for liver transplantation in Europe as well as North America. Chronic HDV disease affects 15 million persons worldwide, about 70,000 of whom are in the U.S. The Centers for Disease Control estimates 1,000 deaths annually in the U.S. due to HDV infection.

The HDV virion is composed of a ribonucleoprotein core and an envelope. The core contains HDV-RNA, and hepatitis delta antigen (HDAg), which is the only protein encoded by this virus. The envelope is formed by the surface antigen protein (hepatitis B surface antigen, or HBsAg) of the helper virus, hepatitis B. The envelope is the sole helper function provided by HBV. HDV is able to replicate its RNA within cells in the absence of HBV, but requires HBsAg for packaging and release of HDV virions, as well as for infectivity. As a result of the dependence of HDV on HBV, HDV infects individuals only in association with HBV.

Hepatitis E virus (HEV) is the causative agent of hepatitis E, a form of acute viral hepatitis that is endemic to many resource-limited regions of the world. It is estimated that about 2 billion people, which is about a third of the world population, live in areas endemic for HEV and are at risk for infection. In these areas, hepatitis E is the major form of acute hepatitis; in India for example about 50% of acute hepatitis is due to HEV.

HEV is a small non-enveloped virus with a size of 27-34 nm and is classified as a Hepevirus in the family Hepeviridae. The HEV genome is a single-stranded RNA of 7.2 kb that is positive-sense, with a 5′-methylguanine cap and a 3′ poly(A) stretch, and contains three partially overlapping open reading frames (ORFs)-called orf1, orf2 and orf3. HEV orf1, a polyprotein of 1693 amino acids, encodes the viral nonstructural functions. Functional domains identified in the HEV nonstructural polyprotein include (starting from the N-terminal end)-methyltransferase (MeT), papain-like cysteine protease (PCP), RNA helicase (Hel) and RNA dependent RNA polymerase (RdRp). HEV orf2 encodes a viral capsid protein of 660 amino acids, which is believed to encapsidate the viral RNA genome. HEV orf3 is believed to express a 114 amino acid protein that is dispensable for replication in vitro and is believed to function as a viral accessory protein, likely affecting the host response to infection.

GBV-C, or hepatitis G virus (HGV), like HCV, belongs to the Flaviviridae family. GBV-C has a global distribution, with a high prevalence in the United States donor population, and can be spread by transfusion of contaminated blood and sexual contact, similar to HCV and HBV. Currently, GBV-C can be diagnosed only by detecting its RNA in the serum by polymerase chain reaction. The clinical significance of GBV-C infection with respect to acute or chronic hepatitis is not well understood, but the preponderance of other evidence suggests that GBV-C does not cause hepatitis in humans. The genome of the virus is represented by single-chain RNA with positive polarity. The GBV-C genome is similar to HCV RNA in its organization, i.e. the structural genes are located at the genomic 5′ region and non-structural genes are at the 3′ end. The untranslated region at the 5′ end may serve as an internal ribosomal embarkation site, which ensures translation of an RNA coding region.

### Hepatitis B Virus (HBV)

As used herein “hepatitis B virus” or “HBV” refers to a specific virus of the hepadnaviridae family. HBV is a small (e.g., 3.2 kb) hepatotropic DNA virus that encodes four open reading frames and seven proteins. The seven proteins encoded by HBV include small (S), middle (M), and large (L) surface antigens (HBsAg) or envelope (Env) proteins, pre-core protein, core protein, viral polymerase (Pol), and HBx protein. HBV expresses three surface antigens, or envelope proteins, L, M, and S, with S being the smallest and L being the largest, and M in the middle. The L protein includes the Pre-S1-Pre-S2-S domains, the M protein the Pre-S2-S domains and the S protein only the S domain. Core protein is the subunit of the viral nucleocapsid. Pol is needed for synthesis of viral DNA (reverse transcriptase, RNaseH, and primer), which takes place in nucleocapsids localized to the cytoplasm of infected hepatocytes. PreCore is the core protein with an N-terminal signal peptide and is proteolytically processed at its N and C termini before secretion from infected cells, as the so-called hepatitis B e-antigen (HBeAg). HBx protein is required for efficient transcription of covalently closed circular DNA (cccDNA). HBx is not a viral structural protein. All viral proteins of HBV have their own mRNA except for core and polymerase, which share an mRNA. With the exception of the protein pre-Core, none of the HBV viral proteins are subject to post-translational proteolytic processing.

The HBV virion contains a viral envelope, nucleocapsid, and single copy of the partially double-stranded DNA genome. The nucleocapsid comprises 120 dimers of core protein and is covered by a lipid membrane embedded with the S, M, and L viral envelope or surface antigen proteins. After entry into the cell, the virus is uncoated and the capsid-containing relaxed circular DNA (rcDNA) with covalently bound viral polymerase migrates to the nucleus. During that process, phosphorylation of the core protein induces structural changes, exposing a nuclear localization signal enabling interaction of the capsid with so-called importins. These importins mediate binding of the core protein to nuclear pore complexes upon which the capsid disassembles and polymerase/rcDNA complex is released into the nucleus. Within the nucleus the rcDNA becomes deproteinized (removal of polymerase) and is converted by host DNA repair machinery to a covalently closed circular DNA (cccDNA) genome (further converted into a minichromosome with the addition of histones and other host factors) from which overlapping transcripts encode for HBeAg, HBsAg, Core protein, viral polymerase and HBx protein. Core protein, viral polymerase, and pre-genomic RNA (pgRNA) associate in the cytoplasm and self-assemble into immature pgRNA-containing capsid particles, which further convert into mature rcDNA-capsids and function as a common intermediate that is either enveloped and secreted as infectious virus particles or transported back to the nucleus to replenish and maintain a stable cccDNA pool.

To date, HBV is divided into four serotypes (adr, adw, ayr, ayw) based on antigenic epitopes present on the envelope proteins, and into eight genotypes (A, B, C, D, E, F, G, and H) based on the sequence of the viral genome. Two new genotypes, I and J, have recently been identified. The HBV genotypes are distributed over different geographic regions. For example, the most prevalent genotypes in Asia are genotypes B and C. Genotype D is dominant in Africa, the Middle East, and India, whereas genotype A is widespread in Northern Europe, sub-Saharan Africa, and West Africa.

As used herein, a patient or subject having a “chronic HBV infection” “chronic hepatitis B virus (CHB) infection”, “CHB”, “CHB infection” or “CHB virus infection” refers to the ordinary meaning in the art, more particularly to a patient or subject chronically infected with HBV and having detectable HBsAg (with or without HBeAg) in the blood for six or more months after HBV detection. CHB infection can be classified into four phases which typically, but not always, progress from one to the next: (I) HBeAg-positive chronic infection (previously known as immune tolerant), (II) HBeAg-positive chronic hepatitis (previously known as immune active), (III) HBeAg-negative chronic infection (previously known as inactive carrier), and (IV) HBeAg-negative chronic hepatitis (previously known as reactivation. The different phases of chronic HBV infection can also be characterized by differences in viral load, liver enzyme levels (necroinflammatory activity), HBeAg, or HBsAg load or presence of antibodies to these antigens. cccDNA levels in untreated subjects stay relatively constant at approximately 10 to 50 copies per cell, but may be as low as 1 to 2 copies per cell when suppressed by nucleos(t)ide analogue therapy, even though viremia can vary considerably. The persistence of the cccDNA species leads to chronicity. In some embodiment, a chronic HBV infection can be characterized the laboratory criteria published by the Centers for Disease Control and Prevention (CDC), such as: (i) negative for IgM antibodies to hepatitis B core antigen (IgM anti-HBc) and positive for hepatitis B surface antigen (HBsAg), hepatitis B e antigen (HBeAg), or nucleic acid test for hepatitis B virus DNA, or (ii) positive for HBsAg or nucleic acid test for HBV DNA, or positive for HBeAg two times at least 6 months apart.

In some embodiments of the application, a target nucleic acid sequence is within an HBV genome. In some embodiments, a TALE DNA binding domain is capable of binding to a target nucleic acid sequence within an HBV genome. In some embodiments of the application, a target nucleic acid sequence is within a nucleic acid sequence that encodes an HBV antigen. In some embodiments, a TALE DNA binding domain is capable of binding to a target nucleic acid sequence within a nucleic acid sequence that encodes an HBV antigen. In some embodiments, a first and second half-site sequence of a target nucleic acid sequence is within a nucleic acid sequence that encodes an HBV antigen. In some embodiments, a TALE DNA binding domain is capable of binding to a half-site sequence of a target nucleic acid sequence within an HBV genome. A TALEN monomer comprising a TALE DNA binding domain capable of binding to a target nucleic acid sequence within an HBV genome is also referred to as an “HBV TALEN” throughout the application. A nucleic acid sequence of an HBV genome of any genotype can be a target nucleic acid sequence for TALE DNA binding. In some embodiments, the target nucleic acid sequence is within an HBV genome of one or more of genotypes A, B, C, D, E, F, G, H, I and J. In some embodiments, the HBV genome is of one or more of genotypes A, B, C, D, E, F, G and H.

In some embodiments of the application, the target nucleic acid sequence is within the nucleic acid sequence that encodes HBV core protein. In particular embodiments, the TALE DNA binding domain is capable of binding to a target nucleic acid sequence within a nucleic acid sequence that encodes HBV core protein. In some embodiments, a first and second half-site sequence of the target nucleic acid sequence is within the nucleic acid sequence that encodes HBV core protein.

In some embodiments of the application, the target nucleic acid sequence is within the nucleic acid sequence that encodes HBV polymerase (pol). In particular embodiments, the TALE DNA binding domain is capable of binding to a target nucleic acid sequence within a nucleic acid sequence that encodes HBV pol. In some embodiments, a first and second half-site sequence of the target nucleic acid sequence is within the nucleic acid sequence that encodes HBV pol.

In some embodiments of the application, the target nucleic acid sequence is within the nucleic acid sequence that encodes an HBV surface antigen (HBsAg). In particular embodiments, the TALE DNA binding domain is capable of binding to a target nucleic acid sequence within a nucleic acid sequence that encodes HBsAg. In some embodiments, a first and second half-site sequence of the target nucleic acid sequence is within the nucleic acid sequence that encodes HBsAg.

### TALENs

Transcription Activator-Like Effector Nucleases (TALENs) are artificial restriction enzymes generated by fusing the TAL effector DNA binding domain to a DNA cleavage domain. These reagents enable efficient, programmable, and specific DNA cleavage and represent powerful tools for genome editing in situ. Transcription activator-like effectors (TALEs) can be quickly engineered to bind practically any DNA sequence. The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left TALEN and a right TALEN, which references the handedness of DNA. See U.S. Ser. No. 12/965,590; U.S. Ser. No. 13/426,991 (U.S. Pat. No. 8,450,471); U.S. Ser. No. 13/427,040 (U.S. Pat. No. 8,440,431); U.S. Ser. No. 13/427,137 (U.S. Pat. No. 8,440,432); and U.S. Ser. No. 13/738,381, all of which are incorporated by reference herein in their entirety.

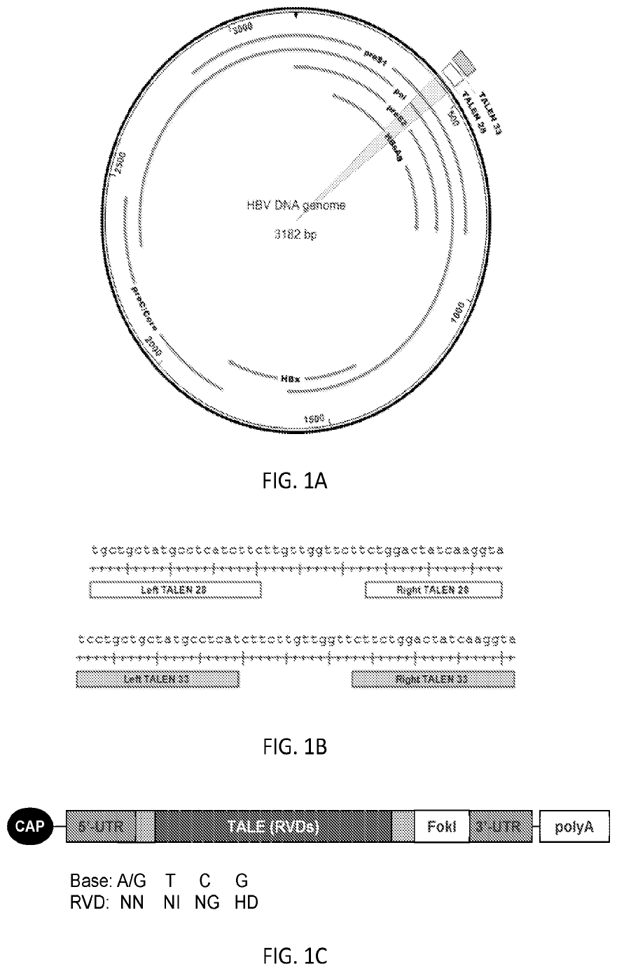

TAL effectors are proteins secreted by plant pathogenic Xanthomonas bacteria. The DNA binding domain contains highly conserved repeat segments of 33-34 amino acid sequence with the exception of the 12th and 13th amino acids. These two locations are highly variable (termed the Repeat Variable Diresidue (RVD)) and show a strong correlation with specific nucleotide recognition (See FIG. 1C). This simple relationship between amino acid sequence and DNA recognition has allowed for the engineering of specific DNA binding domains by selecting a combination of repeat segments containing the appropriate RVDs. Slight changes in the RVD and the incorporation of “nonconventional” RVD sequences can improve targeting specificity.

The non-specific DNA cleavage domain from the end of the FokI endonuclease can be used to construct hybrid nucleases that are active in a yeast assay. These reagents are also active in plant cells and in animal cells. Initial TALEN studies used the wild-type FokI cleavage domain, but some subsequent TALEN studies also used FokI cleavage domain variants with mutations designed to improve cleavage specificity and cleavage activity. The FokI domain functions as a dimer, requiring two TALEN constructs with unique DNA binding domains for sites in the target genome with proper orientation and spacing. Both the number of amino acid residues between the TALE DNA binding domain and the FokI cleavage domain (C-terminal domain) and the number of bases between the two individual TALEN binding sites (spacer) are parameters for achieving high levels of activity. The number of amino acid residues in the C-terminal domain, between the TALE DNA binding domain and the FokI cleavage domain, may be modified by introducing or deleting amino acids according to the number of bases in the spacer sequence. The spacer in the DNA target sequence can be from about 12 to about 30 nucleotides.

The relationship between amino acid sequence and DNA recognition of the TALE binding domain allows for designable proteins. Once the TALEN genes have been assembled they can, for example, be inserted into plasmids or viral constructs, which are then used to transfect the target cell where the gene products are expressed and enter the nucleus to access the genome. TALENs can be used to edit genomes by inducing double-strand breaks (DSBs) in the DNA. Any method and any combination of methods can be used to express TALENs in target cells and effect genome editing, for example. In some embodiments, DNA that includes one or more TALEN genes, such as plasmid DNA, is transfected into target cells. Cells can be transfected with one or more plasmids including one or more TALEN genes. In some embodiments, target cells are transduced with viral vectors delivering TALEN genes. Cells can be transduced with one or more viral vectors, with each viral vector delivering one or more TALEN genes. In some embodiments, TALEN mRNAs are prepared by in vitro transcription (IVT). In some embodiments, in vitro transcribed TALEN mRNAs are transfected into target cells. Cells can be transfected with one or more mRNAs encoding one or more TALEN genes.

In some embodiments, TAL nuclease monomers bind to one of two DNA half-site sequences of a target nucleic acid sequence. Preferably, the half-site sequences are separated by a spacer sequence. This spacing allows the nuclease catalytic domains to dimerize and create a DSB in the spacer sequence between the half-site sequences. Preferably, a first half-site sequence and a second half-site sequence are different sequences and are separated by a spacer sequence.

In some embodiments, a DSB will induce an indel. An “indel” as used herein refers to a mutation resulting from an insertion, deletion or combination thereof. As will be appreciated by those skilled in the art, an indel in the coding region of a genomic sequence will result in a frameshift mutation, unless the length of the indel is a multiple of three. In some embodiments, a DSB will induce a point mutation. “Point mutation” as used herein refers to the substitution of one of the nucleotides. Preferably, the indel or point mutation is within the spacer sequence of a target nucleic acid sequence.

In some embodiments, cleavage of the target nucleic acid sequence results in decreased expression of a target gene. In some embodiments, the decreased expression of a target gene is a decreased mRNA level. In some embodiments, the decreased expression of a target gene is a decreased protein level or decreased protein functionality. The term “decreased” is generally used herein to mean a statistically significant decreased amount. However, for avoidance of doubt, “decreased” means a decrease by at least 10% as compared to a reference level, for example decreased by at least about 20%, or at least about 30%, or at least about 40%, or at least about 50%, or at least about 60%, or at least about 70%, or at least about 75%, or at least about 80%, or at least about 90%, or up to and including a 100% decrease (i.e., absent level as compared to a reference sample), or any decrease between 10% and 100% as compared to a reference level. The reference level can be, for example, the level of expression in an untreated sample or subject, or a sample or subject with a control treatment. The term “statistically significant” or “significantly” refers to statistical significance and generally means two standard deviations (2SD) below normal, or lower, concentration of the marker. The term refers to statistical evidence for the presence of a difference. It is defined as a probability of making a decision to reject the null hypothesis when the null hypothesis is actually true. The decision is often made using the p-value.

In some embodiments, compositions or combinations of the disclosure comprise a first TALEN monomer comprising a first TALE DNA binding domain and a first nuclease catalytic domain, wherein the first TALE DNA binding domain is capable of binding to a first half-site sequence of a target nucleic acid sequence within an HBV genome, and a second TALEN monomer comprising a second TALE DNA binding domain and a second nuclease catalytic domain, wherein the second TALE DNA binding domain is capable of binding to a second half-site sequence of the target nucleic acid sequence, wherein the first TALEN monomer and the second TALEN monomer are capable of forming a dimer that cleaves the target nucleic acid sequence when the first TALE DNA binding domain binds to the first half-site sequence and the second TALE DNA binding domain binds to the second half-site sequence. The first TALEN monomer and the second TALEN monomer can form a dimer through their catalytic domains. Preferably, the first nuclease catalytic domain is a first FokI nuclease catalytic domain and the second nuclease catalytic domain is a second FokI nuclease catalytic domain. Preferably, the first half-site sequence and the second half-site sequence are different sequences and are separated by a spacer sequence.

In some embodiments, the target nucleic acid sequence is within the sequence that encodes HBsAg and HBV polymerase (pol). Preferably, the first half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 1, and the second half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 2; or the first half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 3, and the second half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 4; or the target nucleic acid sequence comprises a polynucleotide sequence at least 90% identical, such as at least 90%, 91%, 92%, 93%, 94%, 95%, 96%, 97%, 98%, 99% or 100% identical, to the nucleic acid sequence of SEQ ID NO: 5 or SEQ ID NO: 6.

In some embodiments, the first and second TALE DNA binding domains each comprise repeat units, and each repeat unit comprises a hypervariable region of two amino acids, known as Repeat Variable Diresidues (RVDs) that determines recognition of a base pair in the target nucleic acid sequence. In some embodiments, the first and second TALE DNA binding domains each comprise from 15 to 20 RVDs, such as 15, 16, 17, 18, 19, or 20 RVDs.

In some embodiments, the first TALE DNA binding domain comprises an amino acid sequence at least 90% identical to the amino acid sequence of SEQ ID NO: 7, such as at least 90%, 91%, 92%, 93%, 94%, 95%, 96%, 97%, 98%, 99% or 100% identical to the amino acid sequence of SEQ ID NO: 7, and the second TALE DNA binding domain comprises an amino acid sequence at least 90% identical to SEQ ID NO: 8, such as at least 90%, 91%, 92%, 93%, 94%, 95%, 96%, 97%, 98%, 99% or 100% identical to the amino acid sequence of SEQ ID NO: 8; or the first TALE DNA binding domain comprises an amino acid sequence at least 90% identical to the amino acid sequence of SEQ ID NO: 9, such as at least 90%, 91%, 92%, 93%, 94%, 95%, 96%, 97%, 98%, 99% or 100% identical to the amino acid sequence of SEQ ID NO: 9, and the second TALE DNA binding domain comprises an amino acid sequence at least 90% identical to SEQ ID NO: 10, such as at least 90%, 91%, 92%, 93%, 94%, 95%, 96%, 97%, 98%, 99% or 100% identical to the amino acid sequence of SEQ ID NO: 10; and the first and second FokI nuclease catalytic domain each comprise an amino acid sequence at least 90% identical to SEQ ID NO: 11, such as at least 90%, 91%, 92%, 93%, 94%, 95%, 96%, 97%, 98%, 99% or 100% identical, to the amino acid sequence of SEQ ID NO: 11. In some embodiments, the first TALE DNA binding domain comprises the amino acid sequence of SEQ ID NO: 7, the second TALE DNA binding domain comprises the amino acid sequence of SEQ ID NO: 8, and the first and second FokI nuclease catalytic domain each comprise the amino acid sequence of SEQ ID NO: 11. In some embodiments, the first TALE DNA binding domain comprises the amino acid sequence of SEQ ID NO: 9, the second TALE DNA binding domain comprises the amino acid sequence of SEQ ID NO: 10, and the first and second FokI nuclease catalytic domain each comprise the amino acid sequence of SEQ ID NO: 11. In some embodiments, the first TALE DNA binding domain consists of the amino acid sequence of SEQ ID NO: 7, the second TALE DNA binding domain consists of the amino acid sequence of SEQ ID NO: 8, and the first and second FokI nuclease catalytic domain each consist of the amino acid sequence of SEQ ID NO: 11. In some embodiments, the first TALE DNA binding domain consists of the amino acid sequence of SEQ ID NO: 9, the second TALE DNA binding domain consists of the amino acid sequence of SEQ ID NO: 10, and the first and second FokI nuclease catalytic domain each consist of the amino acid sequence of SEQ ID NO: 11.

In some embodiments, a first transcription activator like effector nuclease (TALEN) monomer comprises a first TALE DNA binding domain and a first FokI nuclease catalytic domain, wherein the first TALE DNA binding domain is capable of binding to a first half-site sequence of a target nucleic acid sequence within an HBV genome, and the first half-site sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 1 or SEQ ID NO: 3, and a second transcription activator like effector nuclease (TALEN) monomer comprising a second TALE DNA binding domain and a second FokI nuclease catalytic domain, wherein the second TALE DNA binding domain is capable of binding to a second half-site sequence of the target nucleic acid sequence, and the second half-site sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 2 or SEQ ID NO: 4, respectively, wherein the first TALEN monomer and the second TALEN monomer are capable of forming a dimer that cleaves the target nucleic acid sequence when the first TALE DNA binding domain binds to the first half-site sequence and the second TALE DNA binding domain binds to the second half-site sequence. In some embodiments, the target nucleic acid sequence is within an HBV genome of one or more of one or more of genotypes A, B, C, D, E, F, G, H, I, and/or J. In some embodiments, the HBV genome is of one or more of genotypes A, B, C, D, E, F, G, and/or H.

In one aspect, the application provides a method of cleaving a target nucleic acid sequence in an HBV genome, comprising contacting the HBV genome with any composition, combination, nucleic acid molecule, isolated host cell, or pharmaceutical composition described herein. In another aspect, the application provides a method for inducing gene editing of a target nucleic acid sequence in an HBV genome, comprising contacting the HBV genome with any composition, combination, nucleic acid molecule, isolated host cell, or pharmaceutical composition described herein. In some embodiments, the HBV genome is of one or more of genotypes A, B, C, D, E, F, G, H, I and J. In some embodiments, the HBV genome is of one or more of genotypes A, B, C, D, E, F, G, and H.

### Polynucleotides and Vectors

In a general aspect, the application provides a combination of first nucleic acid, preferably a first mRNA molecule, comprising a polynucleotide sequence encoding a first transcription activator like effector nuclease (TALEN) monomer comprising a first TALE DNA binding domain and a first FokI nuclease catalytic domain, wherein the first TALE DNA binding domain is capable of binding to a first half-site sequence of a target nucleic acid sequence within an HBV genome, and the first half-site sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 1; and a second nucleic acid, preferably a second mRNA molecule, comprising a polynucleotide sequence encoding a second transcription activator like effector nuclease (TALEN) monomer comprising a second TALE DNA binding domain and a second FokI nuclease catalytic domain, wherein the second TALE DNA binding domain is capable of binding to a second half-site sequence of the target nucleic acid sequence, and the second half-site sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 2; wherein the first TALEN monomer and the second TALEN monomer are capable of forming a dimer that cleaves the target nucleic acid sequence when the first TALE DNA binding domain binds to the first half-site sequence and the second TALE DNA binding domain binds to the second half-site sequence.

A nucleic acid molecule can comprise any non-naturally occurring polynucleotide sequence encoding an HBV TALEN of the application, which can be made using methods known in the art in view of the present disclosure. A polynucleotide can be in the form of RNA, preferably mRNA, or in the form of DNA obtained by recombinant techniques (e.g., cloning) or produced synthetically (e.g., chemical synthesis). The DNA can be single-stranded or double-stranded, or can contain portions of both double-stranded and single-stranded sequence. The DNA can, for example, comprise genomic DNA, cDNA, or combinations thereof. The polynucleotide can also be a DNA/RNA hybrid. The polynucleotides and vectors of the application can be used for recombinant protein production, expression of the protein in host cell, or the production of viral particles.

In another general aspect, the application provides a combination of mRNA molecules comprising: a first mRNA molecule comprising a polynucleotide sequence encoding a first transcription activator like effector nuclease (TALEN) monomer comprising a first TALE DNA binding domain and a first nuclease catalytic domain, wherein the first TALE DNA binding domain is capable of binding to a first half-site sequence of a target nucleic acid sequence within an HBV genome; and a second mRNA molecule comprising a polynucleotide sequence encoding a second TALEN monomer comprising a second TALE DNA binding domain and a second nuclease catalytic domain, wherein the second TALE DNA binding domain is capable of binding to a second half-site sequence of the target nucleic acid sequence; wherein the first TALEN monomer and the second TALEN monomer are capable of forming a dimer that cleaves the target nucleic acid sequence when the first TALE DNA binding domain binds to the first half-site sequence and the second TALE DNA binding domain binds to the second half-site sequence.

Preferably, the first nuclease catalytic domain is a first FokI nuclease catalytic domain and the second nuclease catalytic domain is a second FokI nuclease catalytic domain. The first and second FokI nuclease catalytic domains can be the same or different. In some embodiments, a FokI nuclease catalytic domain comprises an amino acid sequence at least about 90% identical to the amino acid sequence of SEQ ID NO: 11, such as 90%, 91%, 92%, 93%, 94%, 95%, 96%, 97%, 98%, 99%, or 100% identical to the amino acid sequence of SEQ ID NO: 11. In some embodiments, each FokI nuclease catalytic domain independently comprises an amino acid sequence at least about 90% identical to the amino acid sequence of SEQ ID NO: 11, such as 90%, 91%, 92%, 93%, 94%, 95%, 96%, 97%, 98%, 99%, or 100% identical to the amino acid sequence of SEQ ID NO: 11. In some embodiments, each FokI nuclease catalytic domain consists of the amino acid sequence of SEQ ID NO: 11.

In some embodiments, the first and second mRNA molecules each further comprise one or more, preferably all, of a 5′ cap, a 5′-UTR, a sequence encoding a nuclear localization signal, a sequence encoding an N-terminal domain, a sequence encoding a C-terminal domain, a 3′-UTR, and a poly adenosine tail.

Preferably, an mRNA as described herein comprises a 5′ cap. 5′-ends capped with various groups and their analogues are known in the art. A Cap structure on the 5′-end of mRNAs, which is present in all eukaryotic organisms (and some viruses) is important for stabilizing mRNAs in vivo. Naturally occurring Cap structures comprise a ribo-guanosine residue that is methylated at position N7 of the guanine base. This 7-methylguanosine (m7G) is linked via a 5′- to 5′-triphosphate chain at the 5′-end of the mRNA molecule. The presence of the m7Gppp fragment on the 5′-end is essential for mRNA maturation as it protects the mRNAs from degradation by exonucleases, facilitates transport of mRNAs from the nucleus to the cytoplasm and plays a key role in assembly of the translation initiation complex (Cell 9:645-653, (1976); Nature 266:235, (1977); Federation of Experimental Biologists Society Letter 96:1-11, (1978); Cell 40:223-24, (1985); Prog. Nuc. Acid Res. 35:173-207, (1988); Ann. Rev. Biochem. 68:913-963, (1999); and J Biol. Chem. 274:30337-3040, (1999)).

Only those mRNAs that carry the Cap structure are active in Cap dependent translation; “decapitation” of mRNA results in an almost complete loss of their template activity for protein synthesis (Nature, 255:33-37, (1975); J. Biol. Chem., vol. 253:5228-5231, (1978); and Proc. Natl. Acad. Sci. USA, 72:1189-1193, (1975)).

Another element of eukaryotic mRNA is the presence of 2′-O-methyl nucleoside residues at transcript position 1 (Cap 1), and in some cases, at transcript positions 1 and 2 (Cap 2). The 2′-O-methylation of mRNA provides higher efficacy of mRNA translation in vivo (Proc. Natl. Acad. Sci. USA, 77:3952-3956 (1980)) and further improves nuclease stability of the 5′-capped mRNA. The mRNA with Cap 1 (and Cap 2) is a distinctive mark that allows cells to recognize the bona fide mRNA 5′ end, and in some instances, to discriminate against transcripts emanating from infectious genetic elements (Nucleic Acid Research 43: 482-492 (2015)).

Some examples of 5′ cap structures and methods for preparing mRNAs comprising the same are given in WO2015/051169A2, WO/2015/061491, US 2018/0273576, and U.S. Pat. Nos. 8,093,367, 8,304,529, and 10,487,105. The 5′ cap may be selected from m7GpppA, m7GpppC; unmethylated cap analogs (e.g., GpppG); dimethylated cap analog (e.g., m2,7GpppG), a trimethylated cap analog (e.g., m2,2,7GpppG), dimethylated symmetrical cap analogs (e.g., m7Gpppm7G), or anti reverse cap analogs (e.g., ARCA; m7, 2′OmeGpppG, m72′dGpppG, m7,3′OmeGpppG, m7,3′dGpppG and their tetraphosphate derivatives) (see, e.g., Jemielity, J. et al., RNA 9: 1108-1122 (2003). The 5′ cap may be an ARCA cap (3′-OMe-m7G(5′)pppG). The 5′ cap may be an mCAP (m7G(5′)ppp(5′)G, N7-Methyl-Guanosine-5′-Triphosphate-5′-Guanosine). The 5′ cap may be resistant to hydrolysis. In some embodiments, the 5′ cap is m7GpppAmpG, which is known in the art. In some embodiments, the 5′ cap is m7GpppG or m7GpppGm, which are known in the art. Structural formulas for embodiments of 5′ cap structures are provided below.

In some embodiments, an mRNA described herein comprises a 5′ cap having the structure of Formula (Cap I):

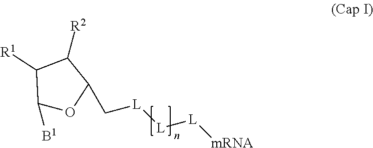

wherein B¹ is a natural or modified nucleobase; R¹ and R² are each independently selected from a halogen, OH, and OCH₃; each L is independently selected from the group consisting of phosphate, phophorothioate, and boranophosphate wherein each L is linked by diester bonds; n is 0 or 1; and mRNA represents an mRNA of the present disclosure linked at its 5′ end. In some embodiments B¹ is G, m⁷G, or A. In some embodiments, n is 0. In some embodiments, n is 1. In some embodiments, B¹ is A or m⁶A and R¹ is OCH₃; wherein G is guanine, m⁷G is 7-methylguanine, A is adenine, and m⁶A is N⁶-methyladenine.

In some embodiments, an mRNA described herein comprises a 5′ cap having the structure of Formula (Cap II):

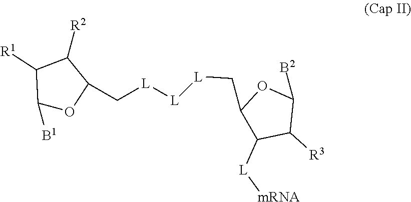

wherein B¹ and B² are each independently a natural or modified nucleobase; R¹, R², and R³ are each independently selected from a halogen, OH, and OCH₃: each L is independently selected from the group consisting of phosphate, phophorothioate, and boranophosphate wherein each L is linked by diester bonds; mRNA represents an mRNA of the present disclosure linked at its 5′ end; and n is 0 or 1. In some embodiments, at least one of R¹, R², and R³ is OH. In some embodiments B¹ is G, m⁷G, or A. In some embodiments, n is 0. In some embodiments, n is 1. In some embodiments, B¹ is A or m⁶A and R¹ is OCH₃; wherein G is guanine, m⁷G is 7-methylguanine, A is adenine, and m⁶A is N⁶-methyladenine.

In some embodiments, an mRNA described herein comprises a 5′ cap having the structure of Formula (Cap III):

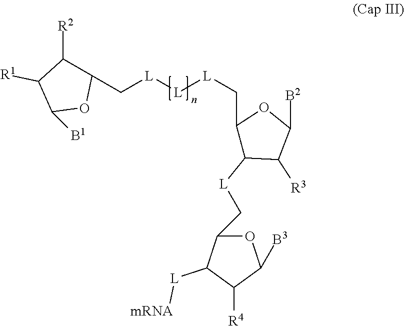

wherein B¹, B², and B³ are each independently a natural or modified nucleobase; R¹, R², R³, and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; n is 0 or 1. In some embodiments, at least one of R¹, R², R³, and R⁴ is OH. In some embodiments B¹ is G, m⁷G, or A. In some embodiments, B¹ is A or m⁶A and R¹ is OCH₃; wherein G is guanine, m⁷G is 7-methylguanine, A is adenine, and m⁶A is N⁶-methyladenine. In some embodiments, n is 1.

In some embodiments, an mRNA described herein comprises a m7GpppG 5′ cap analog having the structure of Formula (Cap IV):

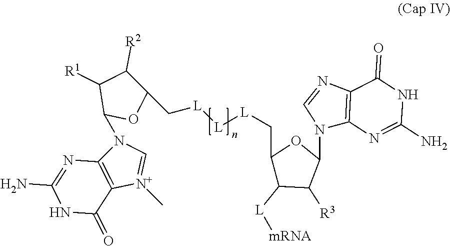

wherein, R¹, R², and R³ are each independently selected from a halogen, OH, and OCH₃. each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R³ is OH. In some embodiments, the 5′ cap is m⁷GpppG wherein R¹, R², and R³ are each OH, n is 1, and each L is a phosphate. In some embodiments, n is 1. In some embodiments, the 5′ cap is m7GpppGm, wherein R¹ and R² are each OH, R³ is OCH₃, each L is a phosphate, mRNA is a CFTR mRNA of the present disclosure linked at its 5′ end, and n is 1.

In some embodiments, an mRNA described herein comprises a m7GpppAmpG 5′ cap analog having the structure of Formula (Cap V):

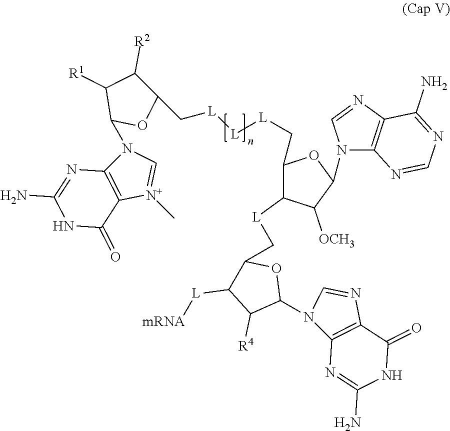

wherein, R¹, R², and R⁴ are each independently selected from a halogen, OH, and OCH₃; each L is independently selected from the group consisting of a phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R⁴ is OH. In some embodiments, the compound of Formula Cap V is m⁷GpppAmpG, wherein R¹, R², and R⁴ are each OH, n is 1, and each L is a phosphate. In some embodiments, n is 1.

Preferably, an mRNA described herein further comprises a 5′ untranslated region (UTR) sequence. As is understood in the art, the 5′- and/or 3′-UTR may affect an mRNA's stability or efficiency of translation. The 5′-UTR may be derived from an mRNA molecule known in the art to be relatively stable (e.g., histone, tubulin, globin, glyceraldehyde 1-phosphate dehydrogenase (GAPDH), actin, or citric acid cycle enzymes) to increase the stability of the translatable oligomer. In other embodiments, a 5′-UTR sequence may include a partial sequence of a cytomegalovirus (CMV) immediate-early 1 (IE1) gene.

Preferably, the 5′-UTR comprises a sequence selected from the 5′-UTRs of human IL-6, alanine aminotransferase 1, human apolipoprotein E, human fibrinogen alpha chain, human transthyretin, human haptoglobin, human alpha-1-antichymotrypsin, human antithrombin, human alpha-1-antitrypsin, human albumin, human beta globin, human complement C3, human complement C5, SynK (thylakoid potassium channel protein derived from the cyanobacteria, Synechocystis sp.), mouse beta globin, mouse albumin, and a tobacco etch virus, or fragments of any of the foregoing. Preferably, the 5′-UTR is derived from a tobacco etch virus (TEV). Preferably, an mRNA described herein comprises a 5′-UTR sequence that is derived from a gene expressed by Arabidopsis thaliana. Preferably, the 5′-UTR sequence of a gene expressed by Arabidopsis thaliana is AT1G58420. Preferred 5′-UTR sequences comprise SEQ ID NOs: 12-17. Preferably the 5′-UTR sequence comprises SEQ ID NO: 13 (AT1G58420).

Preferably an mRNA as described herein comprises a 3′-UTR. Preferably, the 3′-UTR comprises a sequence selected from the 3′-UTRs of alanine aminotransferase 1, human apolipoprotein E, human fibrinogen alpha chain, human haptoglobin, human antithrombin, human alpha globin, human beta globin, human complement C3, human growth factor, human hepcidin, MALAT-1, mouse beta globin, mouse albumin, and Xenopus beta globin, or fragments of any of the foregoing. Preferably, the 3′-UTR is derived from Xenopus beta globin. Preferred 3′-UTR sequences include SEQ ID NOs: 18-24.

Preferably, an mRNA as described herein comprises a 3′ tail region, which can serve to protect the mRNA from exonuclease degradation. The tail region may be a 3′poly(A) and/or 3′poly(C) region. Preferably, the tail region is a 3′poly(A) tail. As used herein a “3′poly(A) tail” or “poly adenosine tail” is a polymer of sequential adenosine nucleotides that can range in size from, for example: 10 to 250 sequential adenosine nucleotides; 60-125 sequential adenosine nucleotides, 90-125 sequential adenosine nucleotides, 95-125 sequential adenosine nucleotides, 95-121 sequential adenosine nucleotides, 100 to 121 sequential adenosine nucleotides, 110-121 sequential adenosine nucleotides; 112-121 sequential adenosine nucleotides; 114-121 adenosine sequential nucleotides; and 115 to 121 sequential adenosine nucleotides. Preferably a 3′ poly adenosine tail as described herein comprise 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125 sequential adenosine nucleotides. 3′ Poly(A) tails can be added using a variety of methods known in the art, e.g., using poly(A) polymerase to add tails to synthetic or in vitro transcribed RNA. Other methods include the use of a transcription vector to encode poly A tails or the use of a ligase (e.g., via splint ligation using a T4 RNA ligase and/or T4 DNA ligase), wherein poly(A) may be ligated to the 3′ end of a sense RNA. Preferably, a combination of any of the above methods is utilized.

In some embodiments, the first and second mRNA molecules each comprise a 5′ cap, a 5′-UTR, a sequence encoding a nuclear localization signal, a sequence encoding an N-terminal domain, a sequence encoding a DNA binding domain, a sequence encoding a nuclease catalytic domain, a sequence encoding a C-terminal domain, a 3′-UTR, and a poly adenosine tail. In some embodiments, the first and second mRNA molecules each comprise a 5′ cap, a 5′-UTR, a sequence encoding an N-terminal domain, a sequence encoding a DNA binding domain, a sequence encoding a nuclease catalytic domain, a sequence encoding a C-terminal domain, a 3′-UTR, and a poly adenosine tail. In some embodiments, the first and second mRNA molecules each comprise a 5′ cap, a 5′-UTR, a sequence encoding a nuclear localization signal, a sequence encoding an N-terminal domain, a sequence encoding a DNA binding domain, a sequence encoding a C-terminal domain, a 3′-UTR, and a poly adenosine tail. In some embodiments, the first and second mRNA molecules each comprise a 5′ cap, a 5′-UTR, a sequence encoding an N-terminal domain, a sequence encoding a DNA binding domain, a sequence encoding a C-terminal domain, a 3′-UTR, and a poly adenosine tail.

The application also relates to a vector comprising a combination of nucleic acids encoding HBV TALENs. As used herein, a “vector” is a nucleic acid molecule used to carry genetic material into another cell, where it can be replicated and/or expressed. Any vector known to those skilled in the art in view of the present disclosure can be used. Examples of vectors include, but are not limited to, plasmids, viral vectors (bacteriophage, animal viruses, and plant viruses), cosmids, and artificial chromosomes (e.g., YACs). Preferably, a vector is a DNA plasmid. A vector can be a DNA vector or an RNA vector. One of ordinary skill in the art can construct a vector of the application through standard recombinant techniques in view of the present disclosure.

A vector of the application can be an expression vector. As used herein, the term “expression vector” refers to any type of genetic construct comprising a nucleic acid coding for an RNA capable of being transcribed. Expression vectors include, but are not limited to, vectors for recombinant protein expression, such as a DNA plasmid or a viral vector, and vectors for delivery of nucleic acid into a subject for expression in a tissue of the subject, such as a DNA plasmid or a viral vector. It will be appreciated by those skilled in the art that the design of the expression vector can depend on such factors as the choice of the host cell to be transformed, the level of expression of protein desired, etc.

Vectors of the application can contain a variety of regulatory sequences. As used herein, the term “regulatory sequence” refers to any sequence that allows, contributes or modulates the functional regulation of the nucleic acid molecule, including replication, duplication, transcription, splicing, translation, stability and/or transport of the nucleic acid or one of its derivatives (i.e., mRNA) into the host cell or organism. In the context of the disclosure, this term encompasses promoters, enhancers and other expression control elements (e.g., polyadenylation signals and elements that affect mRNA stability).

In some embodiments of the application, a vector is a non-viral vector. Examples of non-viral vectors include, but are not limited to, DNA plasmids, bacterial artificial chromosomes, yeast artificial chromosomes, bacteriophages, etc. Preferably, a non-viral vector is a DNA plasmid. A “DNA plasmid”, which is used interchangeably with “DNA plasmid vector,” “plasmid DNA” or “plasmid DNA vector,” refers to a double-stranded and generally circular DNA sequence that is capable of autonomous replication in a suitable host cell. DNA plasmids used for expression of an encoded polynucleotide typically comprise an origin of replication, a multiple cloning site, and a selectable marker, which for example, can be an antibiotic resistance gene. Examples of DNA plasmids suitable that can be used include, but are not limited to, commercially available expression vectors for use in well-known expression systems (including both prokaryotic and eukaryotic systems), such as pSE420 (Invitrogen, San Diego, Calif.), which can be used for production and/or expression of protein in Escherichia coli; pYES2 (Invitrogen, Thermo Fisher Scientific), which can be used for production and/or expression in Saccharomyces cerevisiae strains of yeast; MAXBAC® complete baculovirus expression system (Thermo Fisher Scientific), which can be used for production and/or expression in insect cells; pcDNA™ or pcDNA3™ (Life Technologies, Thermo Fisher Scientific), which can be used for high level constitutive protein expression in mammalian cells; and pVAX or pVAX-1 (Life Technologies, Thermo Fisher Scientific), which can be used for high-level transient expression of a protein of interest in most mammalian cells. The backbone of any commercially available DNA plasmid can be modified to optimize protein expression in the host cell, such as to reverse the orientation of certain elements (e.g., origin of replication and/or antibiotic resistance cassette), replace a promoter endogenous to the plasmid (e.g., the promoter in the antibiotic resistance cassette), and/or replace the polynucleotide sequence encoding transcribed proteins (e.g., the coding sequence of the antibiotic resistance gene), by using routine techniques and readily available starting materials. (See e.g., Sambrook et al., Molecular Cloning a Laboratory Manual, Second Ed. Cold Spring Harbor Press (1989)).

Preferably, a DNA plasmid is an expression vector suitable for protein expression in mammalian host cells. Expression vectors suitable for protein expression in mammalian host cells include, but are not limited to, pcDNA™, pcDNA3™, pVAX, pVAX-1, ADVAX, NTC8454, etc. Preferably, an expression vector is based on pVAX-1, which can be further modified to optimize protein expression in mammalian cells. pVAX-1 is commonly used plasmid in DNA vaccines, and contains a strong human intermediate early cytomegalovirus (CMV-IE) promoter followed by the bovine growth hormone (bGH)-derived polyadenylation sequence (pA). pVAX-1 further contains a pUC origin of replication and kanamycin resistance gene driven by a small prokaryotic promoter that allows for bacterial plasmid propagation.

A vector of the application can also be a viral vector. In general, viral vectors are genetically engineered viruses carrying modified viral DNA or RNA that has been rendered non-infectious, but still contains viral promoters and transgenes, thus allowing for translation of the transgene through a viral promoter. Because viral vectors are frequently lacking infectious sequences, they require helper viruses or packaging lines for large-scale transfection. In certain embodiments, a vector as described herein is, for instance, a recombinant adenovirus, a recombinant retrovirus, a recombinant pox virus such as a vaccinia virus (e.g., Modified Vaccinia Ankara (MVA)), a recombinant alphavirus such as Semliki forest virus, a recombinant paramyxovirus, such as a recombinant measles virus, or another recombinant virus. Examples of viral vectors that can be used include, but are not limited to, adenoviral vectors, adeno-associated virus vectors, pox virus vectors, enteric virus vectors, Venezuelan Equine Encephalitis virus vectors, Semliki Forest Virus vectors, Tobacco Mosaic Virus vectors, lentiviral vectors, etc. In certain embodiments, a vector as described herein is an MVA vector. The vector can also be a non-viral vector.

In some embodiments, a viral vector is an adeno-associated viral (AAV) vector. The term “AAV” includes AAV of any serotype and AAV of any species, such as AAV1, AAV2, AAV3, AAV4, AAV 5, AAV6, AAV7, AAV8, AAV9, AAV10, AAV11, AAV12, rh10, and hybrids thereof, avian AAV, bovine AAV, canine AAV, equine AAV, primate AAV, non-primate AAV, and ovine AAV. In some embodiments, a viral vector is an adenovirus vector, e.g., a recombinant adenovirus vector. A recombinant adenovirus vector can for instance be derived from a human adenovirus (HAdV, or AdHu), or a simian adenovirus such as chimpanzee or gorilla adenovirus (ChAd, AdCh, or SAdV) or rhesus adenovirus (rhAd). Preferably, an adenovirus vector is a recombinant human adenovirus vector, for instance a recombinant human adenovirus serotype 26, or any one of recombinant human adenovirus serotype 5, 4, 35, 7, 48, etc. In other embodiments, an adenovirus vector is a rhAd vector, e.g. rhAd51, rhAd52 or rhAd53. A recombinant viral vector useful for the application can be prepared using methods known in the art in view of the present disclosure. For example, in view of the degeneracy of the genetic code, several nucleic acid sequences can be designed that encode the same polypeptide. A polynucleotide encoding an HBV TALEN of the application can optionally be codon-optimized to ensure proper expression in the host cell (e.g., bacterial or mammalian cells). Codon-optimization is a technology widely applied in the art, and methods for obtaining codon-optimized polynucleotides will be well known to those skilled in the art in view of the present disclosure.

A vector of the application, e.g., a DNA plasmid or a viral vector (particularly AAV, such as AAV7, AAV8, AAV9, or any other AAV with liver tropism, including hybrid AAV, or an adenoviral vector), can comprise any regulatory elements to establish conventional function(s) of the vector, including but not limited to replication and expression of the HBV TALEN(s) encoded by the polynucleotide sequence of the vector. Regulatory elements include, but are not limited to, a promoter, an enhancer, a polyadenylation signal, translation stop codon, a ribosome binding element, a transcription terminator, selection markers, origin of replication, etc. A vector can comprise one or more expression cassettes. An “expression cassette” is part of a vector that directs the cellular machinery to make RNA and protein. An expression cassette typically comprises three components: a promoter sequence, an open reading frame, and a 3′-untranslated region (UTR) optionally comprising a polyadenylation signal. An open reading frame (ORF) is a reading frame that contains a coding sequence of a protein of interest (e.g., HBV TALEN) from a start codon to a stop codon. Regulatory elements of the expression cassette can be operably linked to a polynucleotide sequence encoding an HBV TALEN of interest. As used herein, the term “operably linked” is to be taken in its broadest reasonable context, and refers to a linkage of polynucleotide elements in a functional relationship. A polynucleotide is “operably linked” when it is placed into a functional relationship with another polynucleotide. For instance, a promoter is operably linked to a coding sequence if it affects the transcription of the coding sequence. Any components suitable for use in an expression cassette described herein can be used in any combination and in any order to prepare vectors of the application.

A vector can comprise a promoter sequence, preferably within an expression cassette, to control expression of an HBV TALEN of interest. The term “promoter” is used in its conventional sense, and refers to a nucleotide sequence that initiates the transcription of an operably linked nucleotide sequence. A promoter is located on the same strand near the nucleotide sequence it transcribes. Promoters can be a constitutive, inducible, or repressible. Promoters can be naturally occurring or synthetic. A promoter can be derived from sources including viral, bacterial, fungal, plants, insects, and animals. A promoter can be a homologous promoter (i.e., derived from the same genetic source as the vector) or a heterologous promoter (i.e., derived from a different vector or genetic source). For example, if the vector to be employed is a DNA plasmid, the promoter can be endogenous to the plasmid (homologous) or derived from other sources (heterologous). Preferably, the promoter is located upstream of the polynucleotide encoding an HBV TALEN within an expression cassette.

Examples of promoters that can be used include, but are not limited to, a promoter from simian virus 40 (SV40), a mouse mammary tumor virus (MMTV) promoter, a lentiviral promoter such as the human immunodeficiency virus (HIV) or the bovine immunodeficiency virus (BIV) long terminal repeat (LTR) promoters, a Moloney virus promoter, an avian leukosis virus (ALV) promoter, a cytomegalovirus (CMV) promoter such as the CMV immediate early promoter (CMV-IE), Epstein Barr virus (EBV) promoter, or a Rous sarcoma virus (RSV) promoter. A promoter can also be a promoter from a human gene such as human actin, human myosin, human hemoglobin, human muscle creatine, or human metallothionein. A promoter can also be a tissue specific promoter, such as a muscle or skin specific promoter, natural or synthetic.

A vector can comprise additional polynucleotide sequences that stabilize the expressed transcript, enhance nuclear export of the RNA transcript, and/or improve transcriptional-translational coupling. Examples of such sequences include polyadenylation signals and enhancer sequences. A polyadenylation signal is typically located downstream of the coding sequence for a protein of interest (e.g., an HBV TALEN) within an expression cassette of the vector. Enhancer sequences are regulatory DNA sequences that, when bound by transcription factors, enhance the transcription of an associated gene. An enhancer sequence is preferably located upstream of the polynucleotide sequence encoding an HBV TALEN, but downstream of a promoter sequence within an expression cassette of the vector.

Any polyadenylation signal known to those skilled in the art in view of the present disclosure can be used. For example, the polyadenylation signal can be a SV40 polyadenylation signal, LTR polyadenylation signal, bovine growth hormone (bGH) polyadenylation signal, human growth hormone (hGH) polyadenylation signal, or human 0-globin polyadenylation signal.

Any enhancer sequence known to those skilled in the art in view of the present disclosure can be used. For example, an enhancer sequence can be human actin, human myosin, human hemoglobin, human muscle creatine, or a viral enhancer, such as one from CMV, HA, RSV, or EBV. Examples of particular enhancers include, but are not limited to, Woodchuck HBV Post-transcriptional regulatory element (WPRE), intron/exon sequence derived from human apolipoprotein A1 precursor (ApoAI), untranslated R-U5 domain of the human T-cell leukemia virus type 1 (HTLV-1) long terminal repeat (LTR), a splicing enhancer, a synthetic rabbit β-globin intron, or any combination thereof.

A vector can comprise a polynucleotide sequence encoding a signal peptide sequence for localization of the protein. Preferably, the polynucleotide sequence encoding the signal peptide sequence is located upstream of the polynucleotide sequence encoding an HBV TALEN, for example in the N-terminus region. Preferably, the signal sequence is a nuclear localization signal (NLS) sequence which can direct localization of a TALEN protein to the nucleus to facilitate DNA binding and editing. Any signal peptide known in the art in view of the present disclosure can be used. For example, a signal peptide can be a SV40 Large T-Antigen NLS (PKKKRKV) (SEQ ID NO: 31), nucleoplasmin NLS (KRPAATKKAGQAKKKK) (SEQ ID NO: 32), EGL-13 NLS (MSRRRKANPTKLSENAKKLAKEVEN) (SEQ ID NO: 33), c-Myc proto-oncoprotein NLS (PAAKRVKLD) (SEQ ID NO: 34), or TUS-protein NLS (KLKIKRPVK) (SEQ ID NO: 35).

A vector can comprise a polynucleotide sequence encoding a polypeptide or peptide affinity tag, e.g., glutathione S-transferase (GST), green fluorescent protein (GFP) and other fluorescent proteins such as yellow fluorescent protein (YFP) and mCherry, for example, maltose binding protein, protein A, FLAG tag (e.g., DYKDDDDK (SEQ ID NO: 36) or EYKEEEEK (SEQ ID NO: 37), hexa-histidine (e.g., HHHHHH) (SEQ ID NO: 38), myc tag (e.g., EQKLISEEDL) (SEQ ID NO: 39), influenza HA tag (e.g., YPYDVPDYA) (SEQ ID NO: 40), AcV5 tag (SWKDASGWS) (SEQ ID NO: 41), ALFA-tag (e.g., SRLEEELRRRLTE) (SEQ ID NO: 42), AviTag (e.g., GLNDIFEAQKIEWHE) (SEQ ID NO: 43), E-tag (e.g., GAPVPYPDPLEPR) (SEQ ID NO: 44), S-tag (e.g., KETAAAKFERQHMDS) (SEQ ID NO: 45), Strep-tag (e.g., Strep-tag II: WSHPQFEK) (SEQ ID NO: 46), 17 tag (e.g., MASMTGGQQMG) (SEQ ID NO: 47), Ty1 tag (e.g., EVHTNQDPLD) (SEQ ID NO: 48), V5 tag (e.g., GKPIPNPLLGLDST) (SEQ ID NO: 49), VSV-G tag (e.g., YTDIEMNRLGK) (SEQ ID NO: 50), or Xpress tag (e.g., DLYDDDDK) (SEQ ID NO: 51) in order to facilitate purification and/or detection. The affinity tag or reporter fusion joins the reading frame of the polypeptide of interest (e.g., HBV TALEN) to the reading frame of the gene encoding the affinity tag such that a translational fusion is generated. Expression of the fusion gene results in translation of a single polypeptide that includes both the polypeptide of interest (e.g., HBV TALEN) and the affinity tag. In some instances where affinity tags are utilized, DNA sequence encoding a protease recognition site will be fused between the reading frames for the affinity tag and the polypeptide of interest (e.g., HBV TALEN).

A vector, such as a DNA plasmid, can also include a bacterial origin of replication and an antibiotic resistance expression cassette for selection and maintenance of the plasmid in bacterial cells, e.g., E. coli. Bacterial origins of replication and antibiotic resistance cassettes can be located in a vector in the same orientation as the expression cassette encoding an HBV TALEN, or in the opposite (reverse) orientation. An origin of replication (ORI) is a sequence at which replication is initiated, enabling a plasmid to reproduce and survive within cells. Examples of ORIs suitable for use in the application include, but are not limited to ColE1, pMB1, pUC, pSC101, R6K, and 15A, preferably pUC.

Expression cassettes for selection and maintenance in bacterial cells typically include a promoter sequence operably linked to an antibiotic resistance gene. Preferably, the promoter sequence operably linked to an antibiotic resistance gene differs from the promoter sequence operably linked to a polynucleotide sequence encoding a protein of interest, e.g., HBV TALEN. The antibiotic resistance gene can be codon optimized, and the sequence composition of the antibiotic resistance gene is normally adjusted to bacterial, e.g., E. coli, codon usage. Any antibiotic resistance gene known to those skilled in the art in view of the present disclosure can be used, including, but not limited to, kanamycin resistance gene (Kan^{r}), ampicillin resistance gene (Amp^{r}), and tetracycline resistance gene (Tet^{r}), as well as genes conferring resistance to chloramphenicol, bleomycin, spectinomycin, carbenicillin, etc.

The polynucleotides and expression vectors encoding the HBV TALENs of the application can be made by any method known in the art in view of the present disclosure. For example, a polynucleotide encoding an HBV TALEN can be introduced or “cloned” into an expression vector using standard molecular biology techniques, e.g., polymerase chain reaction (PCR), etc., which are well known to those skilled in the art.

As used herein, the terms “percent identity” or “homology” or “shared sequence identity” or “percent (%) sequence identity” with respect to nucleic acid or polypeptide sequences are defined as the percentage of nucleotide or amino acid residues in the candidate sequence that are identical with the known polynucleotides or polypeptides, after aligning the sequences for maximum percent identity and introducing gaps, if necessary, to achieve the maximum percent homology. N-terminal or C-terminal insertions or deletions shall not be construed as affecting homology, and internal deletions and/or insertions into the nucleotide or polypeptide sequence of less than about 30, less than about 20, or less than about 10 or less than 5 amino acid residues shall not be construed as affecting homology. Homology or identity at the nucleotide or amino acid sequence level can be determined by BLAST (Basic Local Alignment Search Tool) analysis using the algorithm employed by the programs blastp, blastn, blastx, tblastn, and tblastx (Altschul (1997), Nucleic Acids Res. 25, 3389-3402, and Karlin (1990), Proc. Natl. Acad. Sci. USA 87, 2264-2268), which are tailored for sequence similarity searching. The approach used by the BLAST program is to first consider similar segments, with and without gaps, between a query sequence and a database sequence, then to evaluate the statistical significance of all matches that are identified, and finally to summarize only those matches which satisfy a preselected threshold of significance. For a discussion of basic issues in similarity searching of sequence databases, see Altschul (1994), Nature Genetics 6, 119-129. The search parameters for histogram, descriptions, alignments, expect (i.e., the statistical significance threshold for reporting matches against database sequences), cutoff, matrix, and filter (low complexity) can be at the default settings. The default scoring matrix used by blastp, blastx, tblastn, and tblastx is the BLOSUM62 matrix (Henikoff (1992), Proc. Natl. Acad. Sci. USA 89, 10915-10919), recommended for query sequences over 85 in length (nucleotide bases or amino acids).

For blastn, designed for comparing nucleotide sequences, the scoring matrix is set by the ratios of M (i.e., the reward score for a pair of matching residues) to N (i.e., the penalty score for mismatching residues), wherein the default values for M and N can be +5 and −4, respectively. Four blastn parameters can be adjusted as follows: Q=10 (gap creation penalty); R=10 (gap extension penalty); wink=1 (generates word hits at every winkth position along the query); and gapw=16 (sets the window width within which gapped alignments are generated). The equivalent Blastp parameter settings for comparison of amino acid sequences can be: Q=9; R=2; wink=1; and gapw=32. A BESTFIT® comparison between sequences, available in the GCG package version 10.0, can use DNA parameters GAP=50 (gap creation penalty) and LEN=3 (gap extension penalty), and the equivalent settings in protein comparisons can be GAP=8 and LEN=2.

In disclosing the nucleic acid or polypeptide sequences herein, for example sequences of HBV TALEN monomers, TALE DNA binding domains, nuclease catalytic domains, target sequences, spacer sequences, regulatory elements, untranslated regions, enhancer sequences, promoters, also disclosed are sequences considered to be based on or derived from the original sequence. Sequences disclosed therefore include polynucleotide or polypeptide sequences having sequence identities of at least 40%, at least 45%, at least 50%, at least 55%, of at least 60%, at least 65%, at least 70%, at least 75%, at least 80%, or at least 85%, for example at least 86%, at least 87%, at least 88%, at least 89%, at least 90%, at least 91%, at least 92%, at least 93%, at least 94%, at least 95%, at least 96%, at least 97%, at least 98%, at least 99%, or 100% or 85-99% or 85-95% or 90-99% or 95-99% or 97-99% or 98-99% sequence identity with the full-length polynucleotide or polypeptide sequence of any polynucleotide or polypeptide sequence described herein, respectively, such as SEQ ID NOs: 1-55, and fragments thereof. Also disclosed are fragments or portions of any of the sequences disclosed herein. Fragments or portions of sequences can include sequences having at least 5 or at least 7 or at least 10, or at least 20, or at least 30, at least 50, at least 75, at least 100, at least 125, 150 or more or 5-10 or 10-12 or 10-15 or 15-20 or 20-40 or 20-50 or 30-50 or 30-75 or 30-100 amino acid or nucleic acid residues of the entire sequence, or at least 100 or at least 200 or at least 300 or at least 400 or at least 500 or at least 600 or at least 700 or at least 800 or at least 900 or at least 1000 or 100-200 or 100-500 or 100-1000 or 500-1000 amino acid or nucleic acid residues, or any of these amounts but less than 500 or less than 700 or less than 1000 or less than 2000 consecutive amino acids or nucleic acids of any of SEQ ID NOs: 1-55 or of any fragment disclosed herein. Also disclosed are variants of such sequences, e.g., where at least one or two or three or four or five amino acid residues have been inserted N- and/or C-terminal to, and/or within, the disclosed sequence(s) which contain(s) the insertion and substitution, and nucleic acid sequences encoding such variants. Contemplated variants can additionally or alternately include those containing predetermined mutations by, e.g., homologous recombination or site-directed or PCR mutagenesis, and the corresponding polypeptides or nucleic acids of other species, including, but not limited to, those described herein, the alleles or other naturally occurring variants of the family of polypeptides or nucleic acids which contain an insertion and substitution; and/or derivatives wherein the polypeptide has been covalently modified by substitution, chemical, enzymatic, or other appropriate means with a moiety other than a naturally occurring amino acid which contains the insertion and substitution (for example, a detectable moiety such as an enzyme). The nucleic acid sequences described herein can be mRNA sequences.

The term “non-naturally occurring,” “recombinant” or “engineered” nucleic acid molecule or polynucleotide sequence, as used herein, refers to a nucleic acid molecule or non-naturally occurring polynucleotide sequence that has been altered through human intervention. As non-limiting examples, a recombinant nucleic acid molecule: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination) of nucleic acid molecules; 2) includes conjoined nucleotide sequences that are not conjoined in nature, 3) has been engineered using molecular cloning techniques such that it lacks one or more nucleotides with respect to the naturally occurring nucleic acid molecule sequence, and/or 4) has been manipulated using molecular cloning techniques such that it has one or more sequence changes or rearrangements with respect to the naturally occurring nucleic acid sequence. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector.

### Lipid-Based Formulations

Therapies based on the intracellular delivery of nucleic acids to target cells face both extracellular and intracellular barriers. Indeed, naked nucleic acid materials cannot be easily administered systemically due to their toxicity, low stability in serum, rapid renal clearance, reduced uptake by target cells, phagocyte uptake and their ability in activating the immune response, all features that preclude their clinical development. When exogenous nucleic acid material (e.g., mRNA) enters the human biological system, it is recognized by the reticuloendothelial system (RES) as foreign pathogens and cleared from blood circulation before having the chance to encounter target cells within or outside the vascular system. It has been reported that the half-life of naked nucleic acid in the blood stream is around several minutes (Kawabata K, Takakura Y, Hashida M, Pharm Res. 1995 June; 12(6):825-30). Chemical modification and a proper delivery method can reduce uptake by the RES and protect nucleic acids from degradation by ubiquitous nucleases, which increase stability and efficacy of nucleic acid-based therapies. In addition, RNAs or DNAs are anionic hydrophilic polymers that are not favorable for uptake by cells, which are also anionic at the surface. The success of nucleic acid-based therapies thus depends largely on the development of vehicles or vectors that can efficiently and effectively deliver genetic material to target cells and obtain sufficient levels of expression in vivo with minimal toxicity.

Moreover, upon internalization into a target cell, nucleic acid delivery vectors are challenged by intracellular barriers, including endosome entrapment, lysosomal degradation, nucleic acid unpacking from vectors, translocation across the nuclear membrane (for DNA), and release at the cytoplasm (for RNA). Successful nucleic acid-based therapy thus depends upon the ability of the vector to deliver the nucleic acids to the target sites inside of the cells to obtain sufficient levels of a desired activity such as expression of a gene.

While several gene therapies have been able to successfully utilize a viral delivery vector (e.g., AAV), lipid-based formulations have been increasingly recognized as one of the most promising delivery systems for RNA and other nucleic acid compounds due to their biocompatibility and their ease of large-scale production. One of the most significant advances in lipid-based nucleic acid therapies happened in August 2018 when Patisiran (ALN-TTR02) was the first siRNA therapeutic approved by both the Food and Drug Administration (FDA) and the European Commission (EC). ALN-TTR02 is an siRNA formulation based upon the so-called Stable Nucleic Acid Lipid Particle (SNALP) transfecting technology. Despite the success of Patisiran, the delivery of nucleic acid therapeutics, including mRNA, via lipid formulations is still undergoing development.

Some art-recognized lipid-formulated delivery vehicles for nucleic acid therapeutics include, according to various embodiments, polymer based carriers, such as polyethyleneimine (PEI), lipidoid-containing formulations, lipid nanoparticles and liposomes, nanoliposomes, ceramide-containing nanoliposomes, multivesicular liposomes, proteoliposomes, both natural and synthetically-derived exosomes, natural, synthetic and semi-synthetic lamellar bodies, nanoparticulates, micelles, and emulsions.

These lipid formulations vary in their structure and composition, and as can be expected in a rapidly evolving field, several different terms have been used in the art to describe a single type of delivery vehicle. At the same time, the terms for lipid formulations have frequently been conflated throughout the scientific literature, and this inconsistent use has caused confusion as to the exact meaning of several terms for lipid formulations. Among the several potential lipid formulations, liposomes, cationic liposomes, and lipid nanoparticles are specifically described in detail and defined herein for the purposes of the present disclosure.

### Lipid-mRNA Formulations

An mRNA as disclosed herein, pharmaceutically acceptable salts thereof, and/or combinations of nucleic acids encoding HBV TALENS can be incorporated into a lipid formulation (i.e., a lipid-based delivery vehicle).

In the context of the present disclosure, a lipid-based delivery vehicle typically serves to transport a desired mRNA or combination of mRNA molecules to a target cell or tissue. The lipid-based delivery vehicle can be any suitable lipid-based delivery vehicle known in the art. In some embodiments, the lipid-based delivery vehicle is a liposome, a cationic liposome, or a lipid nanoparticle containing an mRNA or combination of mRNA molecules of the present disclosure. In some embodiments, the lipid-based delivery vehicle comprises a nanoparticle or a bilayer of lipid molecules, and an mRNA or combination of mRNA molecules of the present disclosure. In some embodiments, the lipid bilayer preferably further comprises a neutral lipid or a polymer. The term “neutral lipid” means a lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols. In some embodiments, the lipid formulation preferably comprises a liquid medium. In some embodiments, the formulation preferably further encapsulates a nucleic acid. In some embodiments, the lipid formulation preferably further comprises a nucleic acid and a neutral lipid or a polymer. In some embodiments, the lipid formulation preferably encapsulates the nucleic acid.

The description provides lipid formulations comprising one or more therapeutic mRNA molecules encapsulated within the lipid formulation. In some embodiments, the lipid formulation comprises liposomes. In some embodiments, the lipid formulation comprises cationic liposomes. In some embodiments, the lipid formulation comprises lipid nanoparticles.

In some embodiments, the mRNA or combination of mRNA molecules is fully encapsulated within the lipid portion of the lipid formulation such that the mRNA or combination of mRNA molecules in the lipid formulation is resistant in aqueous solution to nuclease degradation. The term “fully encapsulated” means that the nucleic acid (e.g., mRNA) in the nucleic acid-lipid particle is not significantly degraded after exposure to serum or a nuclease assay that would significantly degrade free RNA. When fully encapsulated, preferably less than 25% of the nucleic acid in the particle is degraded in a treatment that would normally degrade 100% of free nucleic acid, more preferably less than 10%, and most preferably less than 5% of the nucleic acid in the particle is degraded. “Fully encapsulated” as used herein also means that the nucleic acid-lipid particles do not rapidly decompose into their component parts upon in vivo administration. In other embodiments, the lipid formulations described herein are substantially non-toxic to mammals such as humans. In some embodiments, the combination of mRNA molecules is encapsulated within the same lipid nanoparticle. In some embodiments, each mRNA molecule in the combination of mRNA molecules is independently encapsulated in individual lipid nanoparticles.

The lipid formulations of the disclosure also typically have a total lipid:RNA ratio (mass/mass ratio) of from about 1:1 to about 100:1, from about 1:1 to about 50:1, from about 2:1 to about 45:1, from about 3:1 to about 40:1, from about 5:1 to about 38:1, or from about 6:1 to about 40:1, or from about 7:1 to about 35:1, or from about 8:1 to about 30:1; or from about 10:1 to about 25:1; or from about 8:1 to about 12:1; or from about 13:1 to about 17:1; or from about 18:1 to about 24:1; or from about 20:1 to about 30:1. In some preferred embodiments, the total lipid:RNA ratio (mass/mass ratio) is from about 10:1 to about 25:1. The ratio may be any value or subvalue within the recited ranges, including endpoints.

The lipid formulations of the present disclosure typically have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm, and are substantially non-toxic. The diameter may be any value or subvalue within the recited ranges, including endpoints. In addition, nucleic acids, when present in the lipid nanoparticles of the present disclosure, are resistant in aqueous solution to degradation with a nuclease.

In preferred embodiments, the lipid formulations comprise an mRNA or combination of mRNA molecules, a cationic lipid (e.g., one or more cationic lipids or salts thereof described herein), a phospholipid, and a conjugated lipid that inhibits aggregation of the particles (e.g., one or more PEG-lipid conjugates). The lipid formulations can also include cholesterol. The term “lipid conjugate” means a conjugated lipid that inhibits aggregation of lipid particles. Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides, cationic PEG lipids, polyoxazoline (POZ)-lipid conjugates, polyamide oligomers, and mixtures thereof. PEG or POZ can be conjugated directly to the lipid or may be linked to the lipid via a linker moiety. Any linker moiety suitable for coupling the PEG or the POZ to a lipid can be used including, e.g., non-ester-containing linker moieties and ester-containing linker moieties. In certain preferred embodiments, non-ester-containing linker moieties, such as amides or carbamates, are used.

The term “cationic lipid” as used herein refers to amphiphilic lipids and salts thereof having a positive, hydrophilic head group; one, two, three, or more hydrophobic (i.e., having apolar groups) fatty acid or fatty alkyl chains; and a connector between these two domains. An ionizable or protonatable cationic lipid is typically protonated (i.e., positively charged) at a pH below its pKₐ and is substantially neutral at a pH above the pKₐ. Preferred ionizable cationic lipids are those having a pKa that is less than physiological pH, which is typically about 7.4. The cationic lipids of the disclosure may also be termed titratable cationic lipids. The cationic lipids can be an “amino lipid” having a protonatable tertiary amine (e.g., pH-titratable) head group. Some amino exemplary amino lipid can include Cis alkyl chains, wherein each alkyl chain independently has 0 to 3 (e.g., 0, 1, 2, or 3) double bonds; and ether, ester, or ketal linkages between the head group and alkyl chains. Such cationic lipids include, but are not limited to, DSDMA, DODMA, DLinDMA, DLenDMA, γ-DLenDMA, DLin-K-DMA, DLin-K-C2-DMA (also known as DLin-C2K-DMA, XTC2, and C2K), DLin-K-C3-DM A, DLin-K-C4-DMA, DLen-C2K-DMA, y-DLen-C2K-DMA, DLin-M-C2-DMA (also known as MC2), DLin-M-C3-DMA (also known as MC3) and (DLin-MP-DMA)(also known as 1-Bl 1).

The term “anionic lipid” as used herein refers to a lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

In the nucleic acid-lipid formulations, the mRNA or combination of mRNA molecules may be fully encapsulated within the lipid portion of the formulation, thereby protecting the nucleic acid from nuclease degradation. In preferred embodiments, a lipid formulation comprising an mRNA or combination of mRNA molecules is fully encapsulated within the lipid portion of the lipid formulation, thereby protecting the nucleic acid from nuclease degradation. In certain instances, the mRNA or combination of mRNA molecules in the lipid formulation is not substantially degraded after exposure of the particle to a nuclease at 37° C. for at least 20, 30, 45, or 60 minutes. In certain other instances, the mRNA or combination of mRNA molecules in the lipid formulation is not substantially degraded after incubation of the formulation in serum at 37° C. for at least 30, 45, or 60 minutes or at least 2, 3, 4, 5, 6, 7, 8, 9, 10, 12, 14, 16, 18, 20, 22, 24, 26, 28, 30, 32, 34, or 36 hours. In other embodiments, the mRNA or combination of mRNA molecules is complexed with the lipid portion of the formulation.

In the context of nucleic acids, full encapsulation may be determined by performing a membrane-impermeable fluorescent dye exclusion assay, which uses a dye that has enhanced fluorescence when associated with a nucleic acid. Encapsulation is determined by adding the dye to a lipid formulation, measuring the resulting fluorescence, and comparing it to the fluorescence observed upon addition of a small amount of nonionic detergent. Detergent-mediated disruption of the lipid layer releases the encapsulated nucleic acid, allowing it to interact with the membrane-impermeable dye. Nucleic acid encapsulation may be calculated as E=(I₀−I)/I₀, where I and I₀ refer to the fluorescence intensities before and after the addition of detergent.

In other embodiments, the present disclosure provides a nucleic acid-lipid composition comprising a plurality of nucleic acid-liposomes, nucleic acid-cationic liposomes, or nucleic acid-lipid nanoparticles. In some embodiments, the nucleic acid-lipid composition comprises a plurality of mRNA-liposomes. In some embodiments, the nucleic acid-lipid composition comprises a plurality of mRNA-cationic liposomes. In some embodiments, the nucleic acid-lipid composition comprises a plurality of mRNA-lipid nanoparticles.

In some embodiments, the lipid formulations comprise mRNA or combination of mRNA molecules that is fully encapsulated within the lipid portion of the formulation, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% (or any fraction thereof or range therein) of the particles have the mRNA or combination of mRNA molecules encapsulated therein. The amount may be any value or subvalue within the recited ranges, including endpoints.

Depending on the intended use of the lipid formulation, the proportions of the components can be varied, and the delivery efficiency of a particular formulation can be measured using assays known in the art.

According to some embodiments, the expressible polynucleotides and mRNA constructs described herein are lipid formulated. The lipid formulation is preferably selected from, but not limited to, liposomes, cationic liposomes, and lipid nanoparticles. In one preferred embodiment, a lipid formulation is a cationic liposome or a lipid nanoparticle (LNP) comprising:
(a) an mRNA or combination of mRNA molecules of the present disclosure,
(b) a cationic lipid,
(c) an aggregation reducing agent (such as polyethylene glycol (PEG) lipid or PEG-modified lipid),
(d) optionally a non-cationic lipid (such as a neutral lipid), and
(e) optionally, a sterol.

Preferably, the lipid nanoparticles encapsulating the mRNA or combination of mRNA molecules comprise a cationic lipid and at least one other lipid selected from the group consisting of anionic lipids, zwitterionic lipids, neutral lipids, steroids, polymer conjugated lipids, phospholipids, glycolipids, and combinations thereof.

In some embodiments, the cationic lipid is an ionizable cationic lipid. In one embodiment, the lipid nanoparticle formulation consists of (i) at least one cationic lipid; (ii) a helper lipid; (iii) a sterol (e.g., cholesterol); and (iv) a PEG-lipid, in a molar ratio of about 20% to about 40% ionizable cationic lipid: about 25% to about 45% helper lipid: about 25% to about 45% sterol; about 0.5-5% PEG-lipid. Example cationic lipids (including ionizable cationic lipids), helper lipids (e.g., neutral lipids), sterols, and ligand-containing lipids (e.g., PEG-lipids) are described herein below.

The selection of specific lipids and their relative % compositions depends on several factors including the desired therapeutic effect, the intended in vivo delivery target, and the planned dosing regimen and frequency. Generally, lipids that correspond to both high potency (i.e, therapeutic effect such as knockdown activity or translation efficiency) and biodegradability resulting in rapid tissue clearance are most preferred. However, biodegradability may be less important for formulations that are intended for only one or two administrations within the subject. In addition, the lipid composition may require careful engineering so that the lipid formulation preserves its morphology during in vivo administration and its journey to the intended target, but will then be able to release the active agent upon uptake into target cells. Thus, several formulations typically need to be evaluated in order to find the best possible combination of lipids in the best possible molar ratio of lipids as well as the ratio of total lipid to active ingredient.

Suitable lipid components and methods of manufacturing lipid nanoparticles are well known in the art and are described for example in PCT/US2020/023442, U.S. Pat. Nos. 8,058,069, 8,822,668, 9,738,593, 9,139,554, PCT/US2014/066242, PCT/US2015/030218, PCT/2017/015886, and PCT/US2017/067756, the contents of which are incorporated by reference.

### Cationic Lipids

The lipid formulation preferably includes a cationic lipid suitable for forming a cationic liposome or lipid nanoparticle. Cationic lipids are widely studied for nucleic acid delivery because they can bind to negatively charged membranes and induce uptake. Generally, cationic lipids are amphiphiles containing a positive hydrophilic head group, two (or more) lipophilic tails, or a steroid portion and a connector between these two domains. Preferably, the cationic lipid carries a net positive charge at about physiological pH. Cationic liposomes have been traditionally the most commonly used non-viral delivery systems for oligonucleotides, including plasmid DNA, antisense oligos, and siRNA/small hairpin RNA-shRNA. Cationic lipids, such as DOTAP, (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids by electrostatic interaction, providing high in vitro transfection efficiency.

In the presently disclosed lipid formulations, the cationic lipid may be, for example, N,N-dioleyl-N,N-dimethylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA·Cl), 1,2-Dilinoleoyl-3-trimethylaminopropane chloride salt (DLin-TAP·Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanediol (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), (6Z,9Z,28Z,31Z)-heptatriaconta-6,9,28 31-tetraen-19-yl 4-(dimethylamino) butanoate (DLin-M-C3-DMA), 3-((6Z,9Z,28Z,31Z)-heptatriaconta-6,9,28,3 1-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), or any combination thereof. Other cationic lipids include, but are not limited to, N,N-distearyl-N,N-dimethylammonium bromide (DDAB), 3P—(N—(N′,N′-dimethylaminoethane)-carbamoyl)cholesterol (DC-Choi), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. No. 8,158,601; and Love et al., PNAS, 107(5), 1864-69, 2010, the contents of which are herein incorporated by reference.

Other suitable cationic lipids include those having alternative fatty acid groups and other dialkylamino groups, including those, in which the alkyl substituents are different (e.g., N-ethyl-N-methylamino-, and N-propyl-N-ethylamino-). These lipids are part of a subcategory of cationic lipids referred to as amino lipids. In some embodiments of the lipid formulations described herein, the cationic lipid is an amino lipid. In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C1 to C22 may be used. Other scaffolds can also be used to separate the amino group and the fatty acid or fatty alkyl portion of the amino lipid.

In some embodiments, the lipid formulation comprises the cationic lipid with Formula I according to the patent application PCT/EP2017/064066. In this context, the disclosure of PCT/EP2017/064066 is also incorporated herein by reference.

In some embodiments, amino or cationic lipids of the present disclosure are ionizable and have at least one protonatable or deprotonatable group, such that the lipid is positively charged at a pH at or below physiological pH (e.g., pH 7.4), and neutral at a second pH, preferably at or above physiological pH. Of course, it will be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the disclosure. In certain embodiments, the protonatable lipids have a pKa of the protonatable group in the range of about 4 to about 11. In some embodiments, the ionizable cationic lipid has a pKa of about 5 to about 7. In some embodiments, the pKa of an ionizable cationic lipid is about 6 to about 7.

In some embodiments, the lipid formulation comprises an ionizable cationic lipid of Formula I:

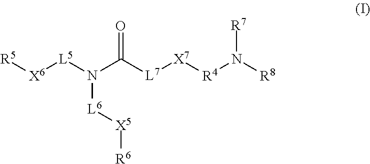

or a pharmaceutically acceptable salt or solvate thereof, wherein R⁵ and R⁶ are each independently selected from the group consisting of a linear or branched C₁-C₃₁ alkyl, C₂-C₃₁ alkenyl or C₂-C₃₁alkynyl and cholesteryl; L⁵ and L⁶ are each independently selected from the group consisting of a linear C₁-C₂₀ alkyl and C₂-C₂₀ alkenyl; X⁵ is —C(O)O—, whereby —C(O)O—R⁶ is formed or —OC(O)— whereby —OC(O)—R⁶ is formed; X⁶ is —C(O)O— whereby —C(O)O—R⁵ is formed or —OC(O)— whereby —OC(O)—R⁵ is formed; X⁷ is S or O; L⁷ is absent or lower alkyl; R⁴ is a linear or branched C₁-C₆ alkyl; and R⁷ and R⁸ are each independently selected from the group consisting of a hydrogen and a linear or branched C₁-C₆ alkyl.

In some embodiments, X⁷ is S.

In some embodiments, X⁵ is —C(O)O—, whereby —C(O)O—R⁶ is formed and X⁶ is —C(O)O— whereby —C(O)O—R is formed.

In some embodiments, R⁷ and R⁸ are each independently selected from the group consisting of methyl, ethyl and isopropyl.

In some embodiments, L⁵ and L⁶ are each independently a C₁-C₁₀ alkyl. In some embodiments, L⁵ is C₁-C₃ alkyl, and L⁶ is C₁-C₅ alkyl. In some embodiments, L⁶ is C₁-C₂ alkyl. In some embodiments, L⁵ and L⁶ are each a linear C₇ alkyl. In some embodiments, L⁵ and L⁶ are each a linear C₉ alkyl.

In some embodiments, R⁵ and R⁶ are each independently an alkenyl. In some embodiments, R⁶ is alkenyl. In some embodiments, R⁶ is C₂-C₉ alkenyl. In some embodiments, the alkenyl comprises a single double bond. In some embodiments, R⁵ and R⁶ are each alkyl. In some embodiments, R⁵ is a branched alkyl. In some embodiments, R⁵ and R⁶ are each independently selected from the group consisting of a C₉ alkyl, C₉ alkenyl and C₉ alkynyl. In some embodiments, R⁵ and R⁶ are each independently selected from the group consisting of a C₁₁ alkyl, C₁₁ alkenyl and C₁₁ alkynyl. In some embodiments, R⁵ and R⁶ are each independently selected from the group consisting of a C₇ alkyl, C₇ alkenyl and C₇ alkynyl. In some embodiments, R⁵ is —CH((CH₂)ₚCH₃)₂ or —CH((CH₂)ₚCH₃)((CH₂)ₚ₋₁CH₃), wherein p is 4-8. In some embodiments, p is 5 and L⁵ is a C₁-C₃ alkyl. In some embodiments, p is 6 and L⁵ is a C3 alkyl. In some embodiments, p is 7. In some embodiments, p is 8 and L⁵ is a C₁-C₃ alkyl. In some embodiments, R⁵ consists of —CH((CH₂)ₚCH₃)((CH₂)ₚ₋₁CH₃), wherein p is 7 or 8.

In some embodiments, R⁴ is ethylene or propylene. In some embodiments, R is n-propylene or isobutylene.

In some embodiments, L⁷ is absent, R⁴ is ethylene, X⁷ is S and R⁷ and R⁸ are each methyl. In some embodiments, L⁷ is absent, R⁴ is n-propylene, X⁷ is S and R⁷ and R⁸ are each methyl. In some embodiments, L⁷ is absent, R⁴ is ethylene, X⁷ is S and R⁷ and R⁸ are each ethyl.

In some embodiments, X⁷ is S, X⁵ is —C(O)O—, whereby —C(O)O—R⁶ is formed, X⁶ is —C(O)O— whereby —C(O)O—R⁵ is formed, L⁵ and L⁶ are each independently a linear C₃-C₇ alkyl, L⁷ is absent, R⁵ is —CH((CH₂)ₚCH₃)₂, and R⁶ is C₇-C₁₂ alkenyl. In some further embodiments, p is 6 and R⁶ is C₉ alkenyl.

In some embodiments, the lipid formulation comprises an ionizable cationic lipid selected from the group consisting of

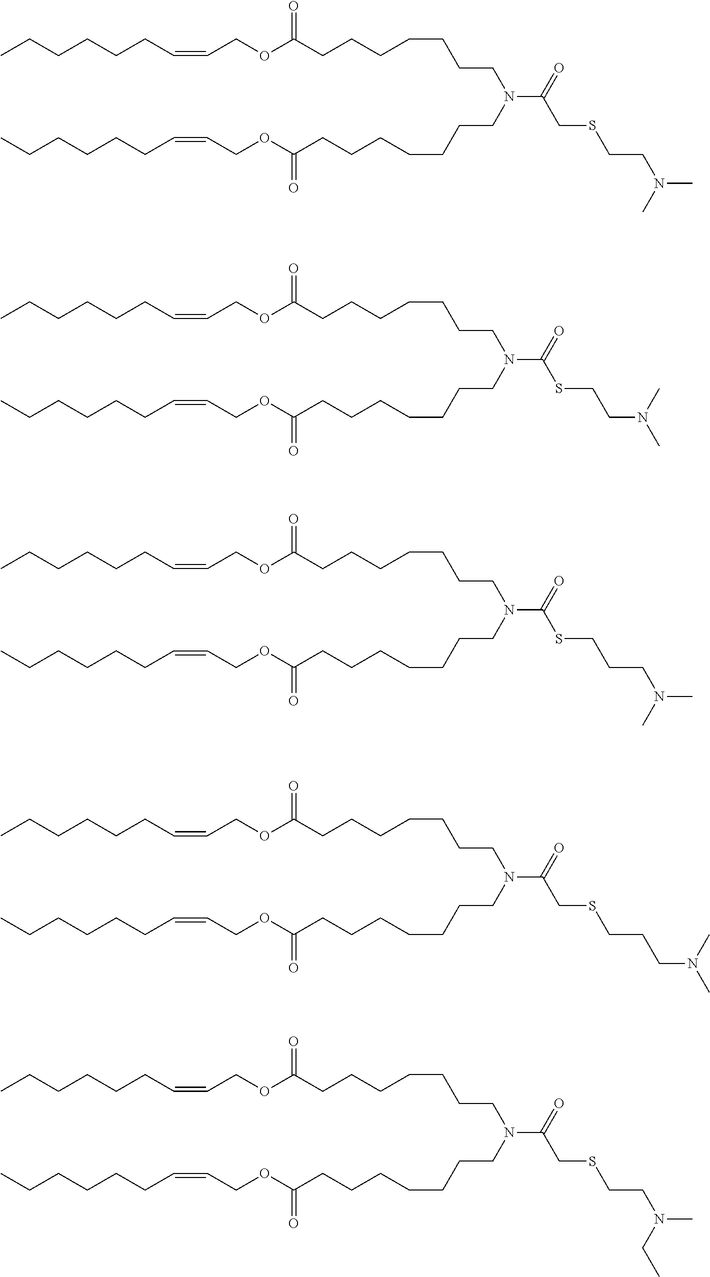

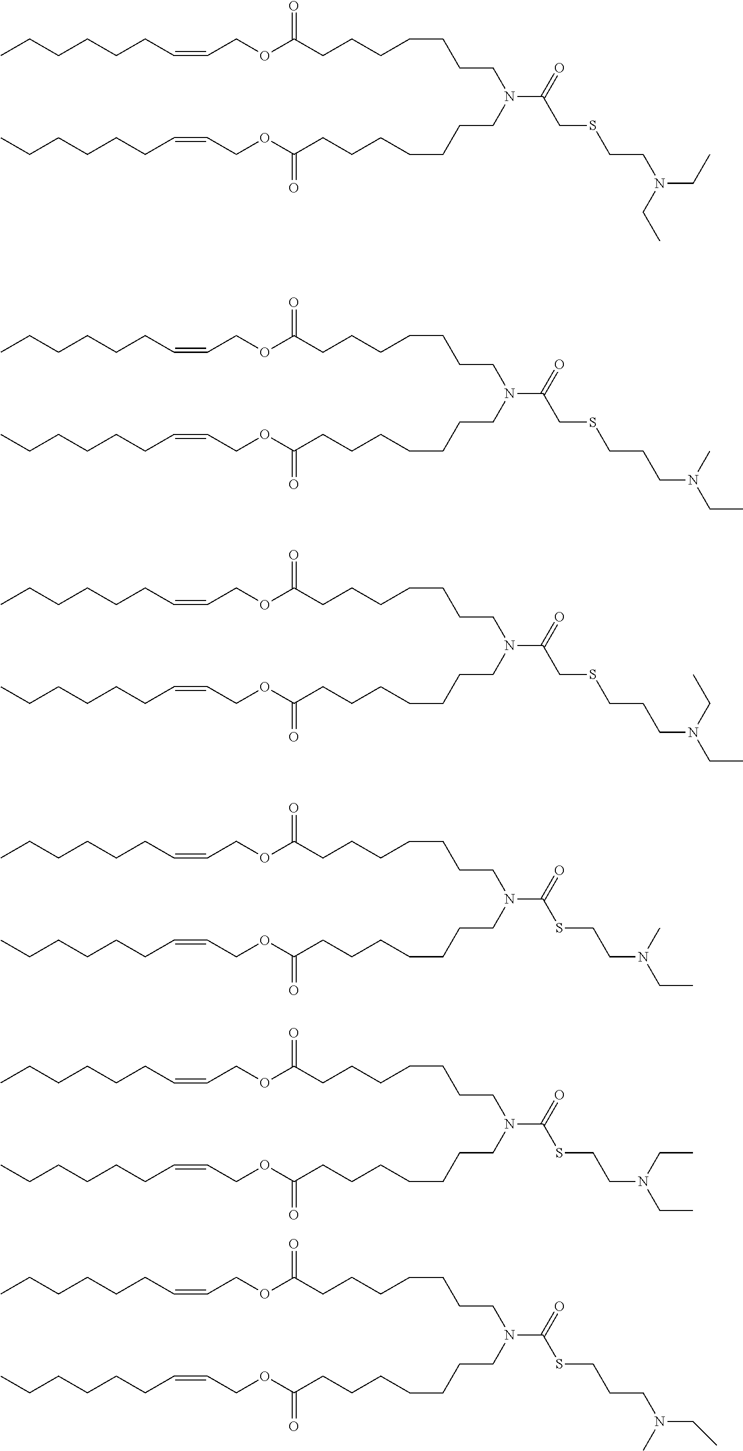

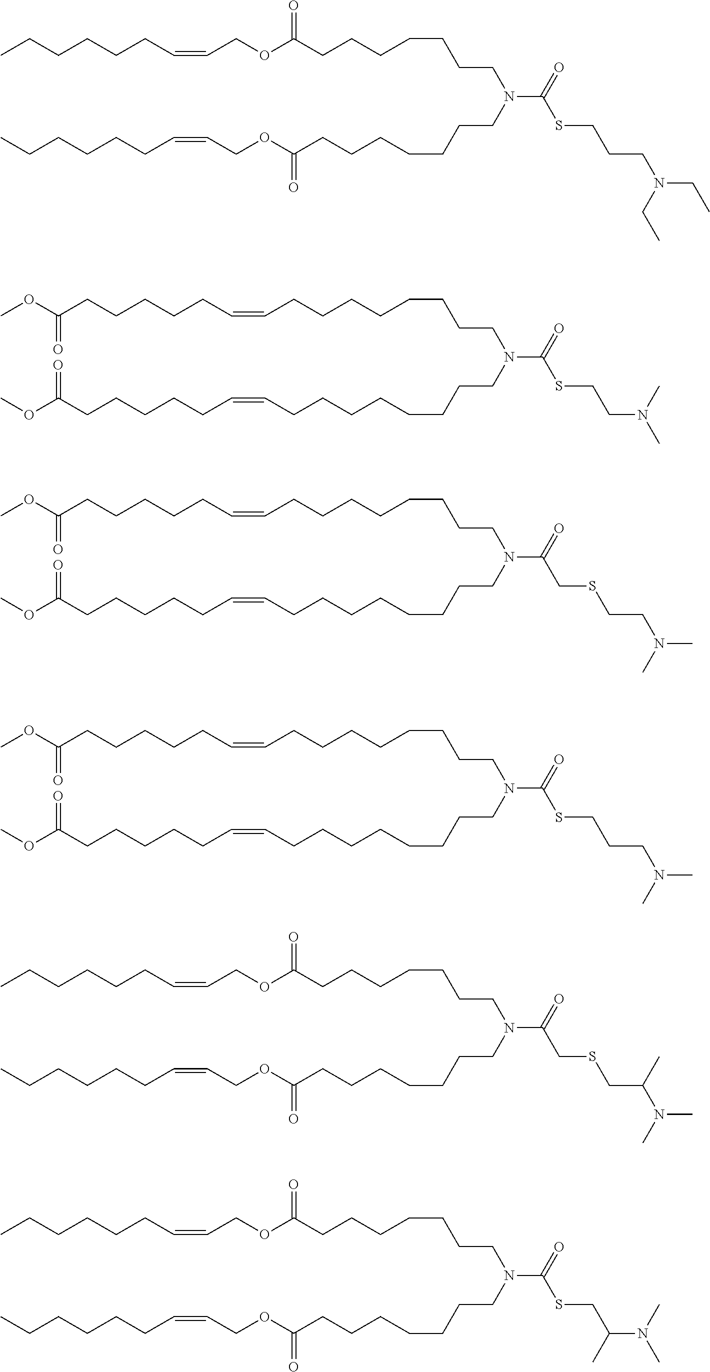

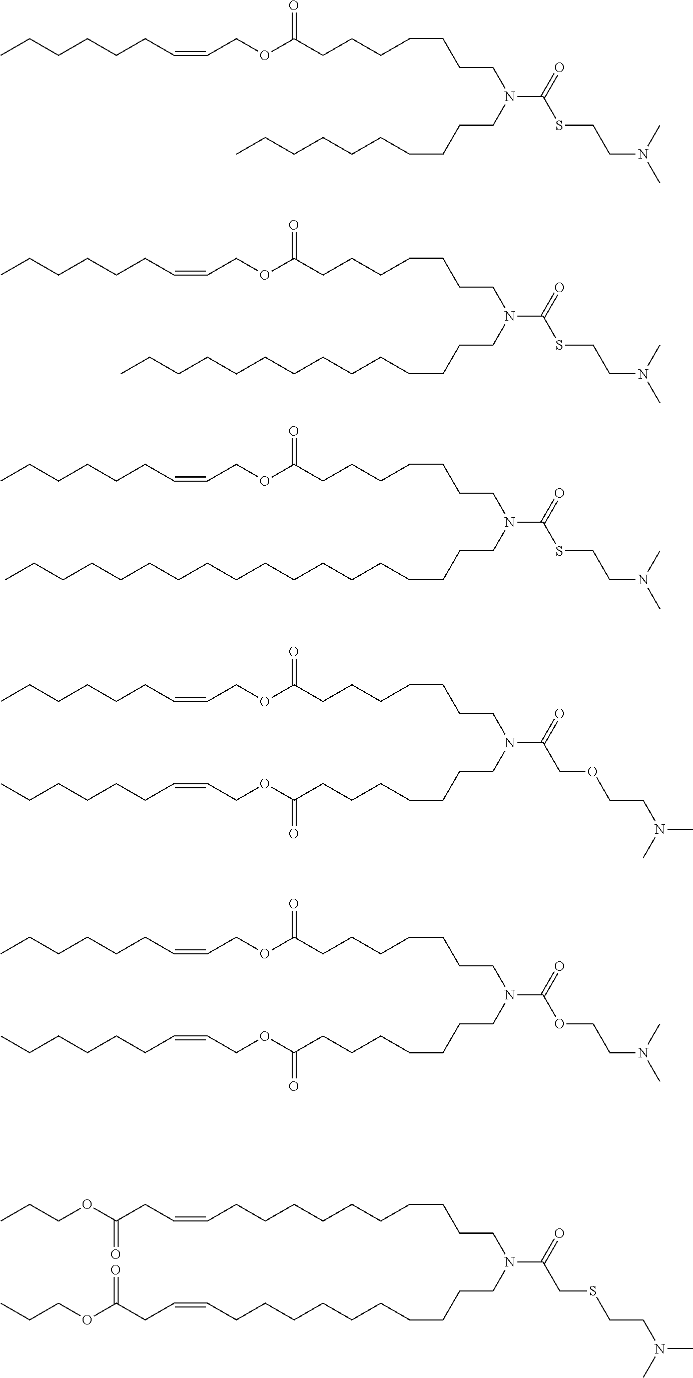

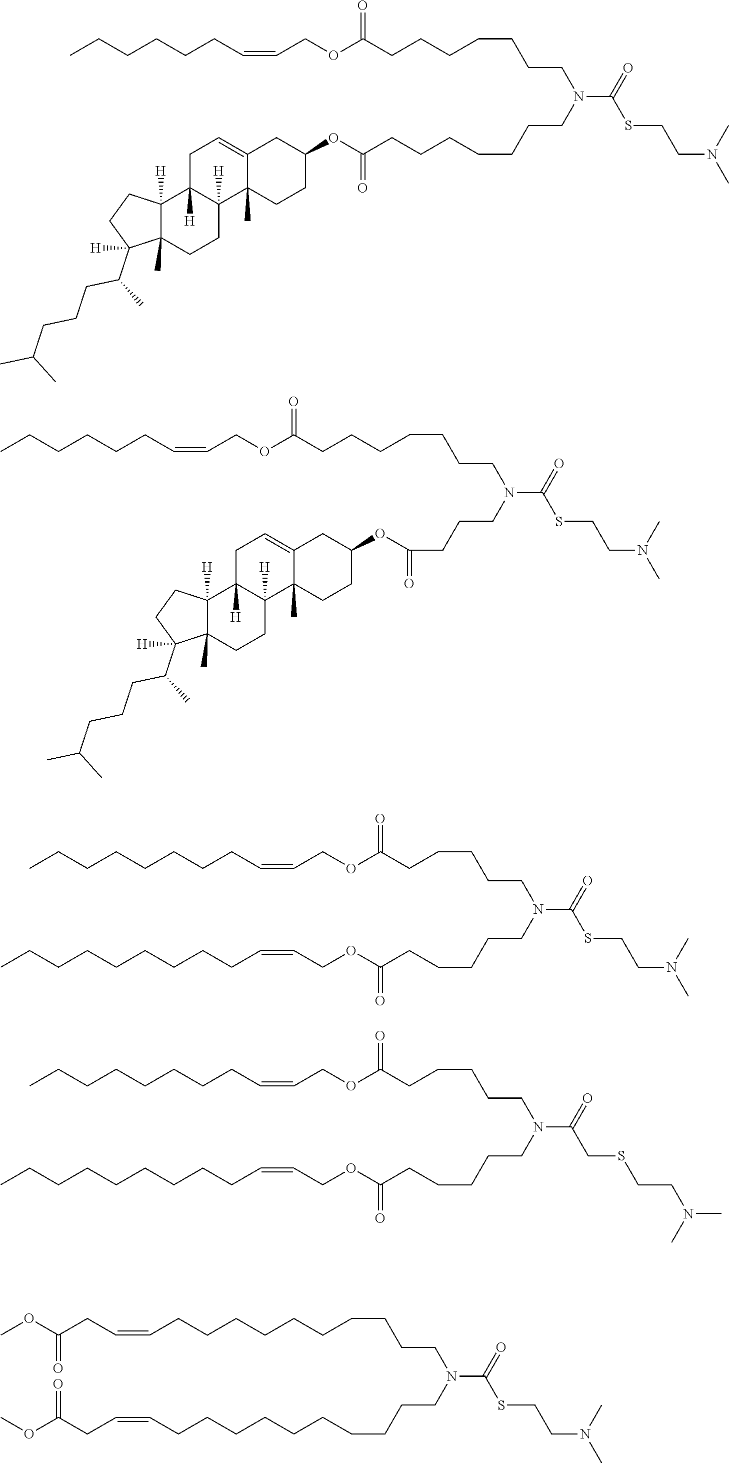

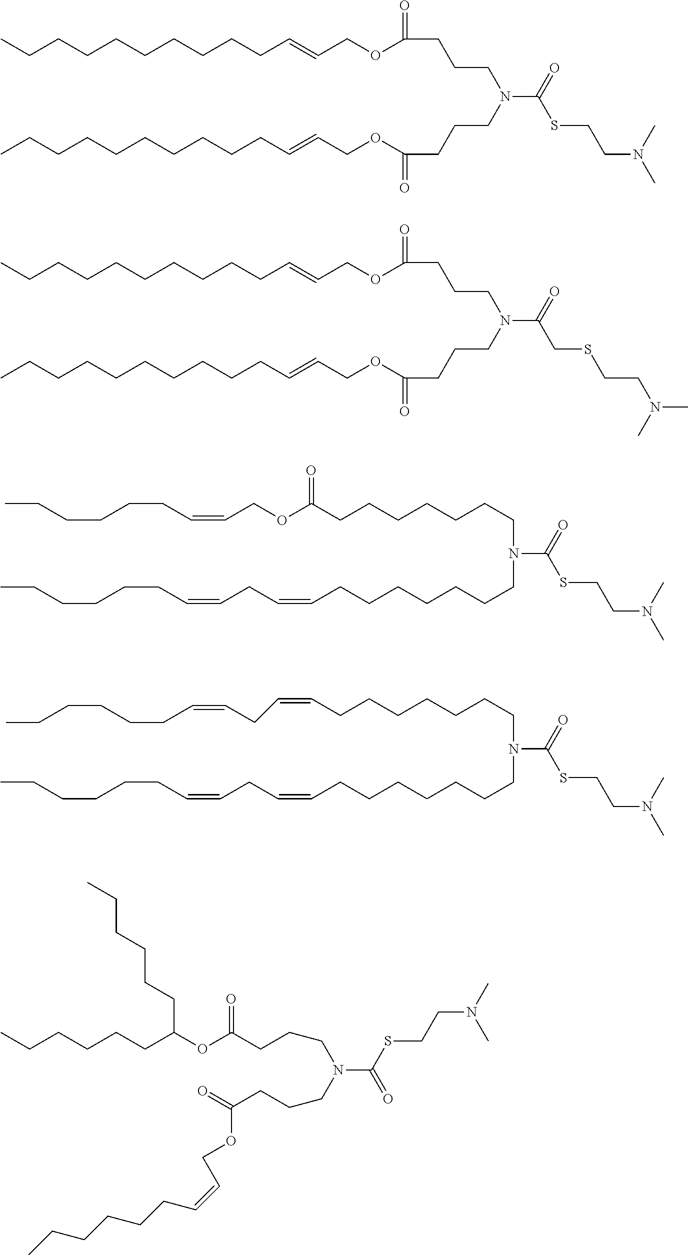

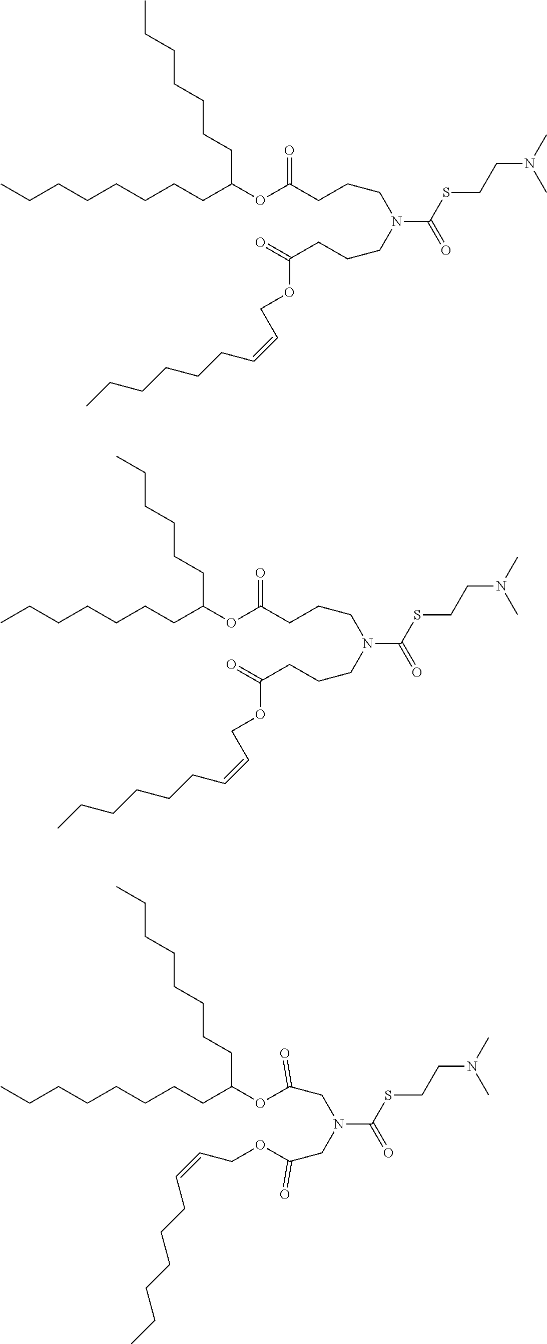

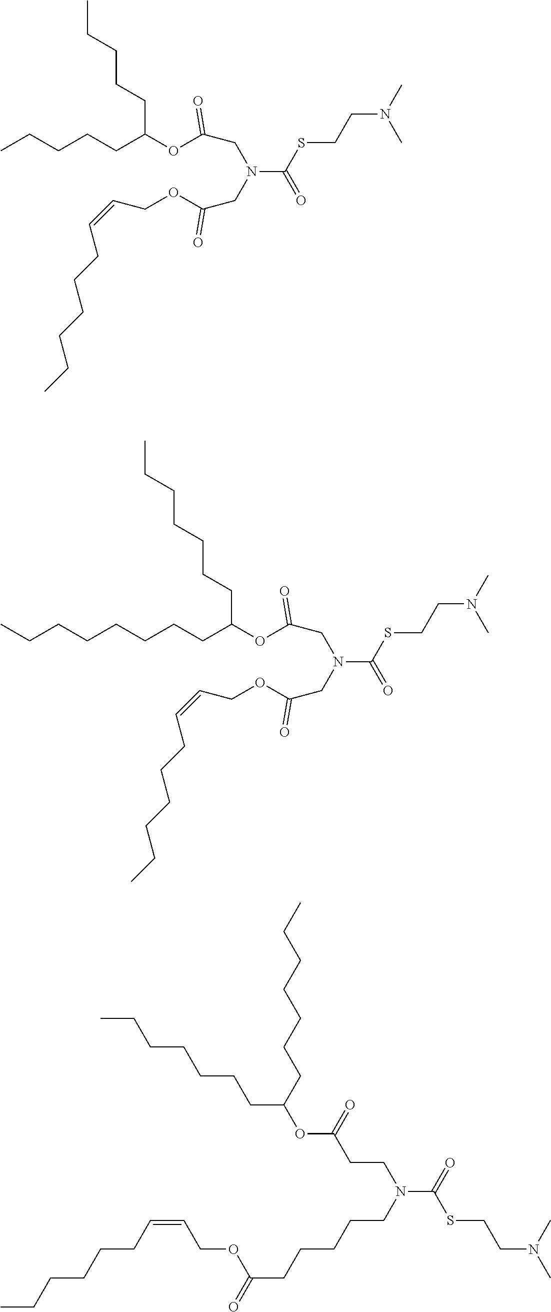

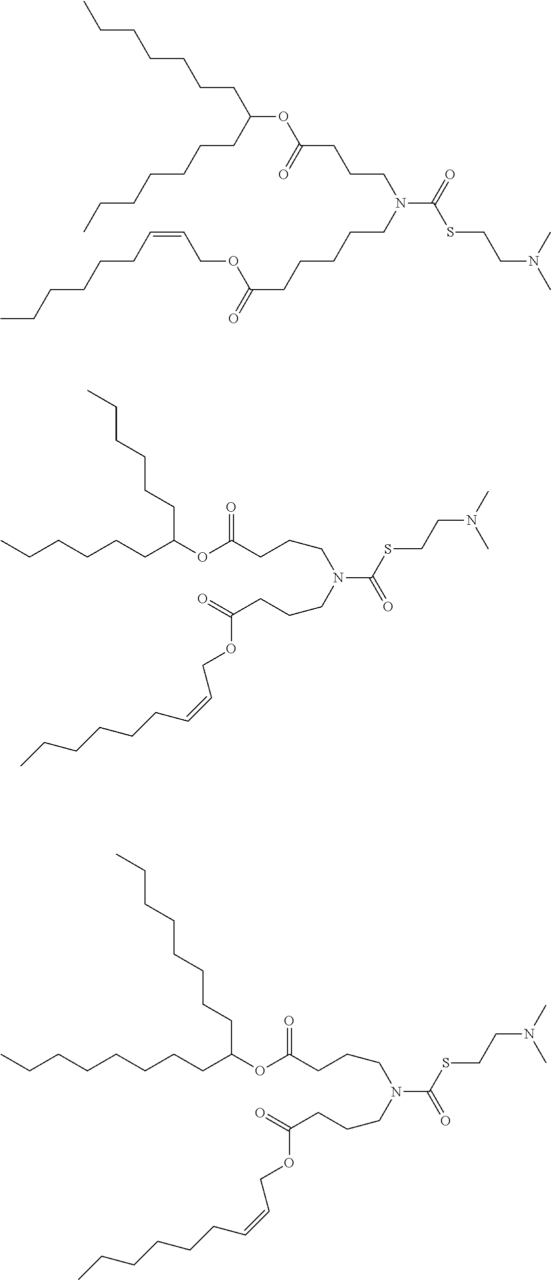

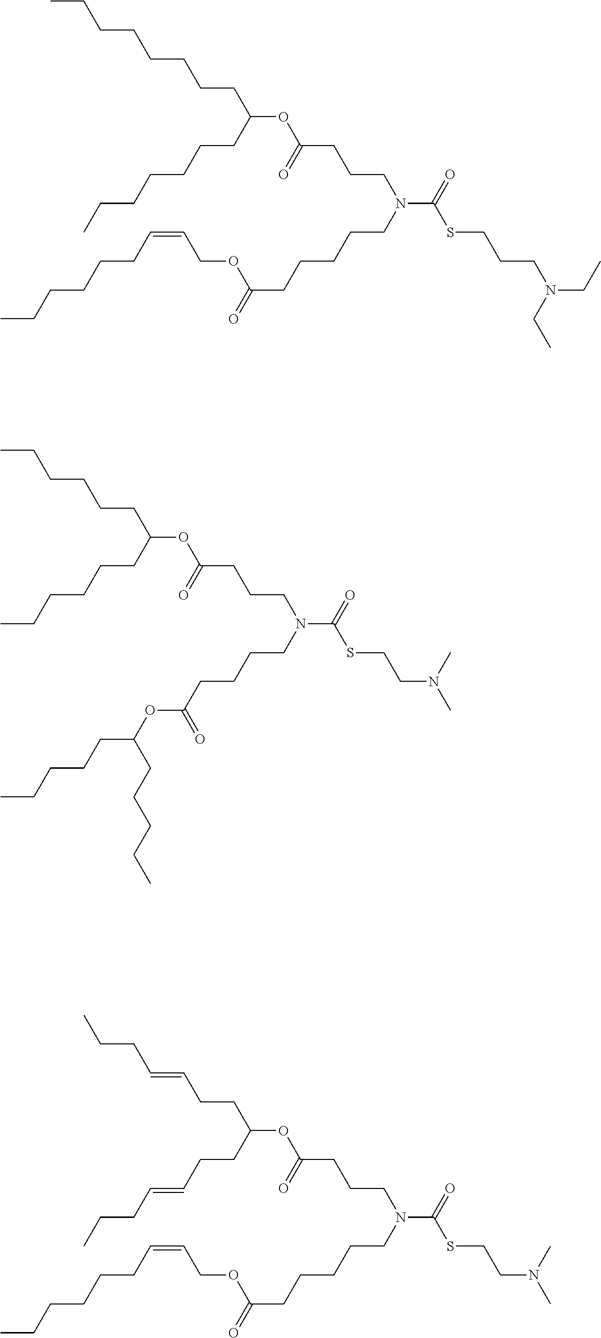

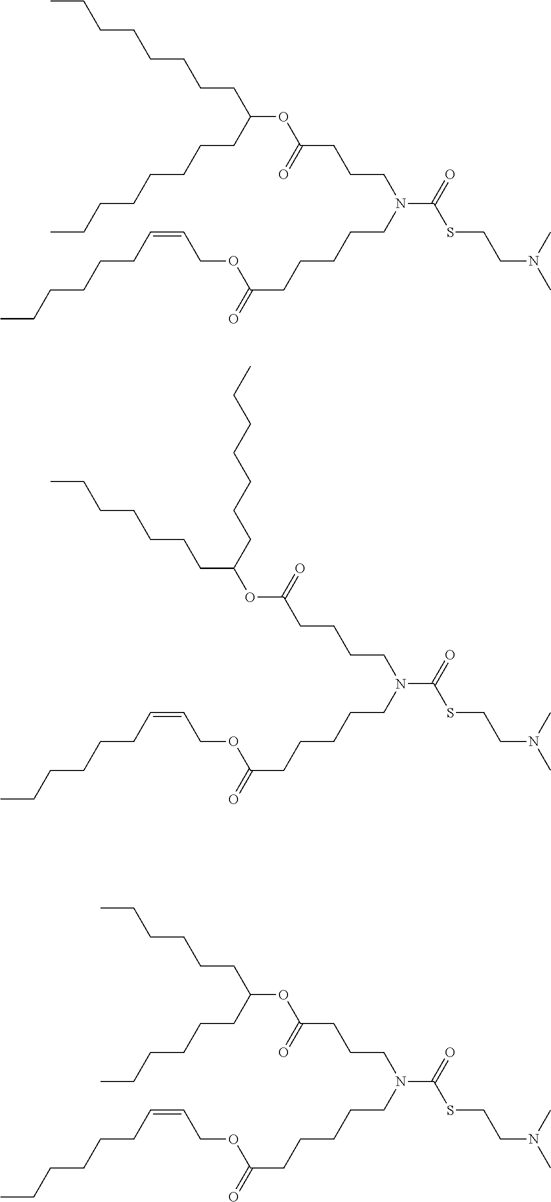

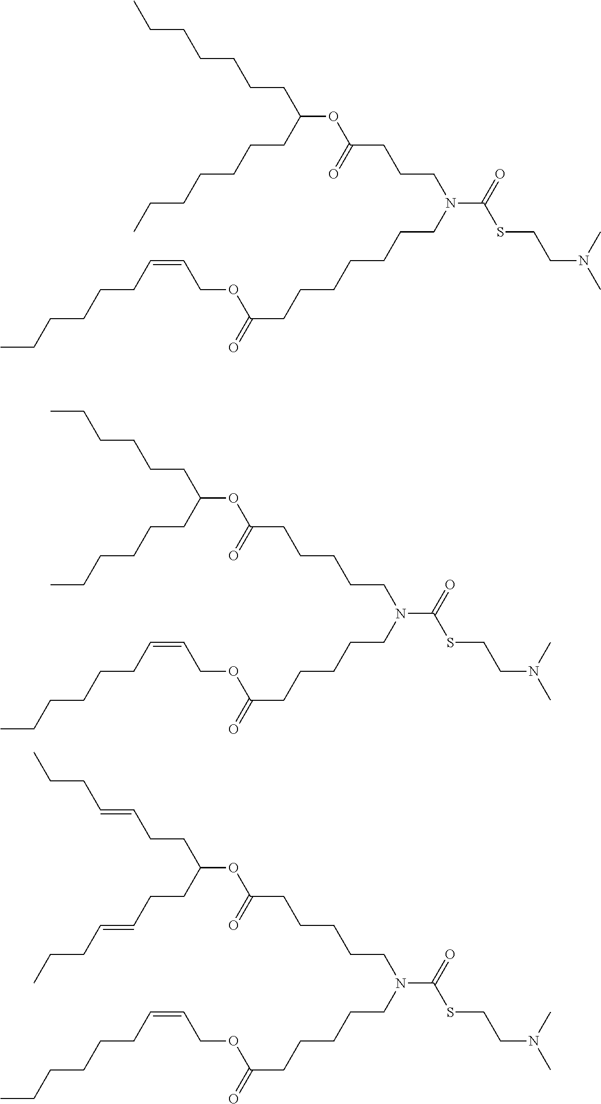

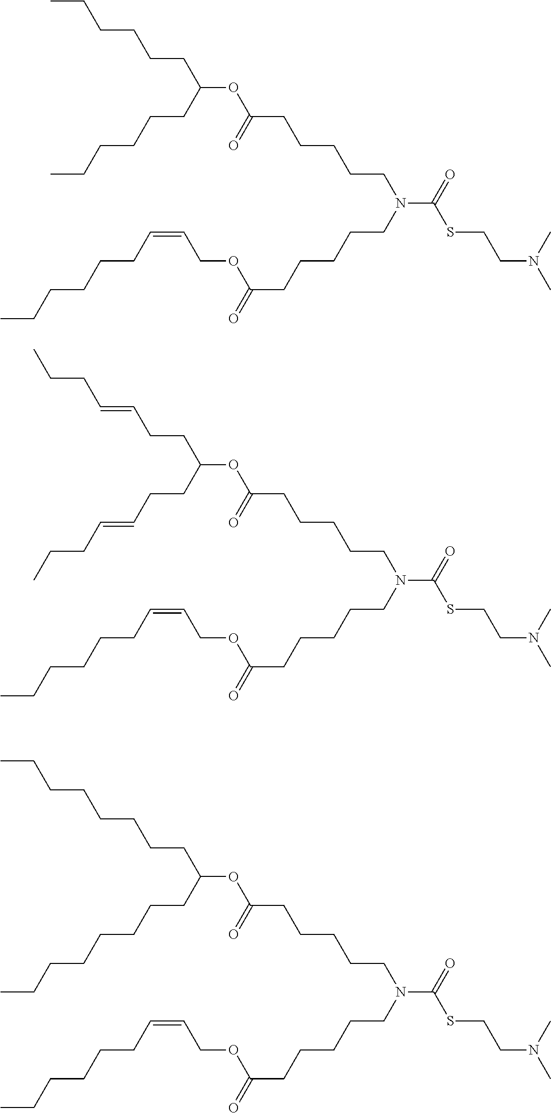

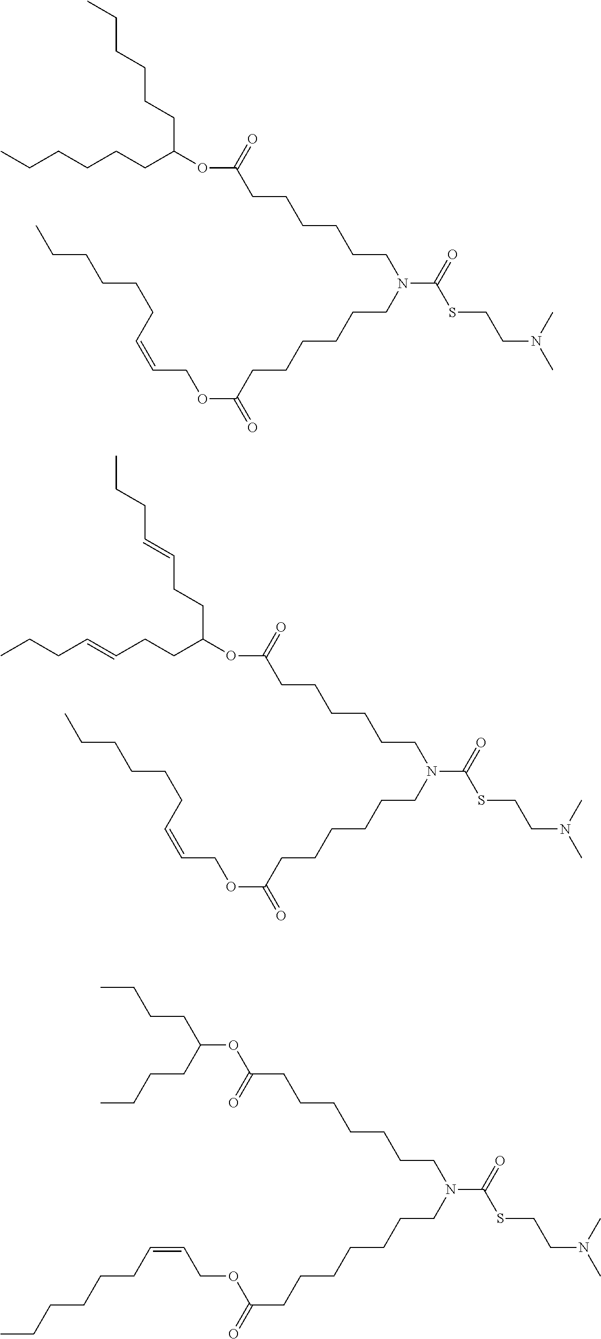

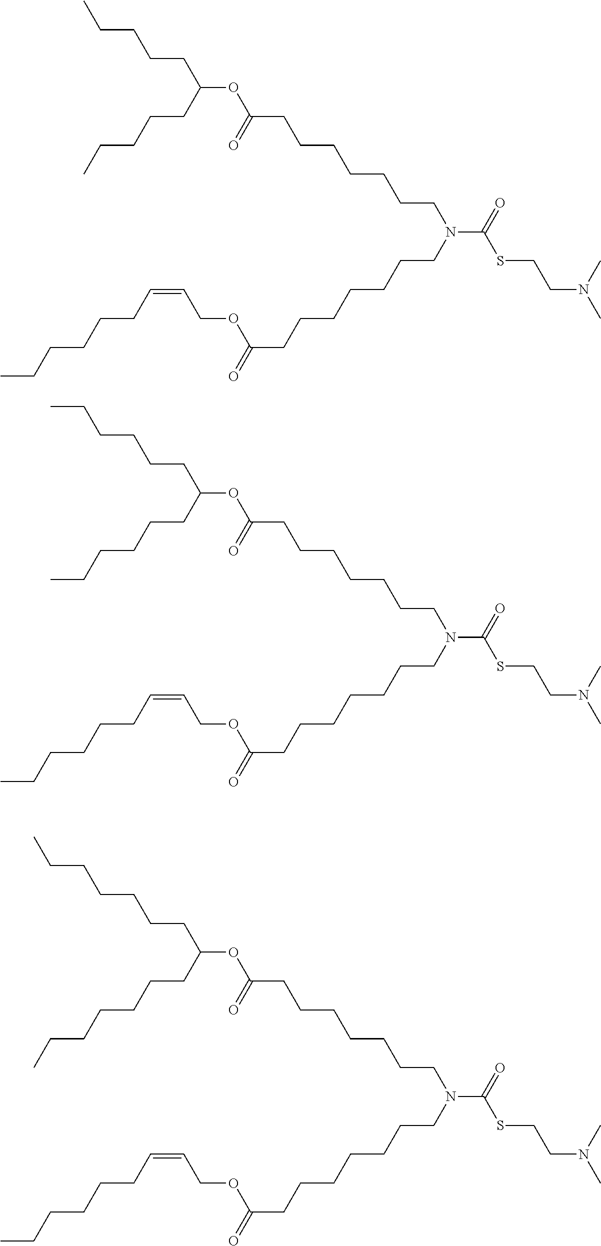

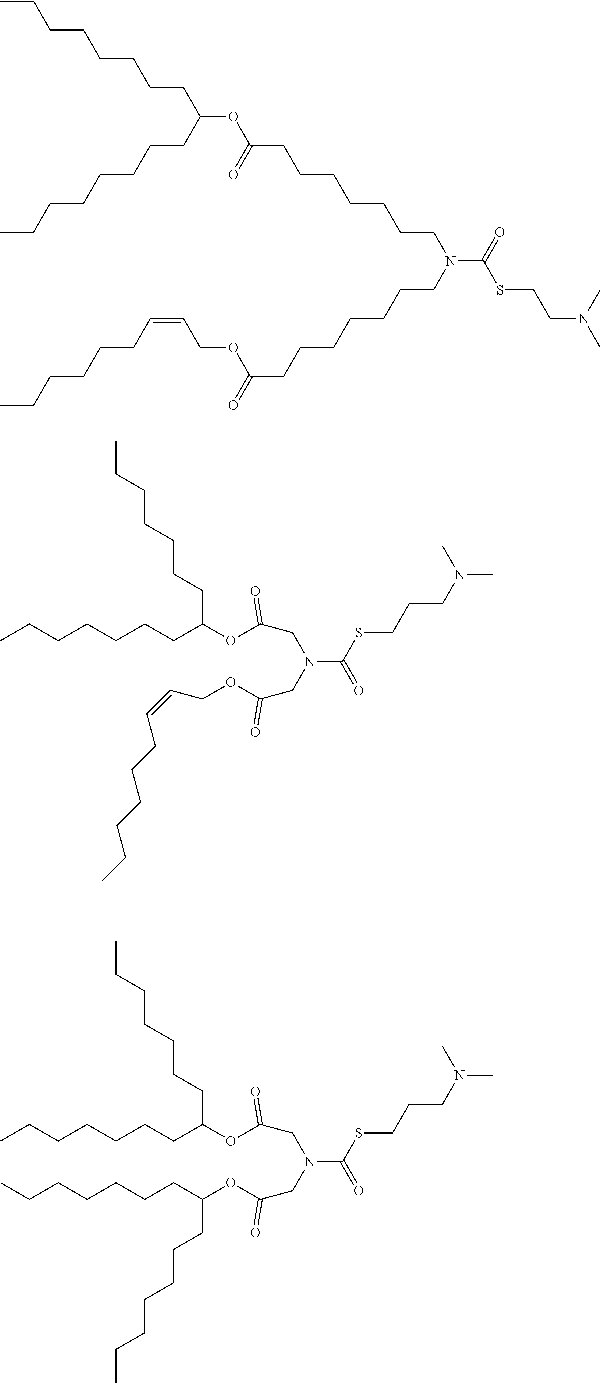

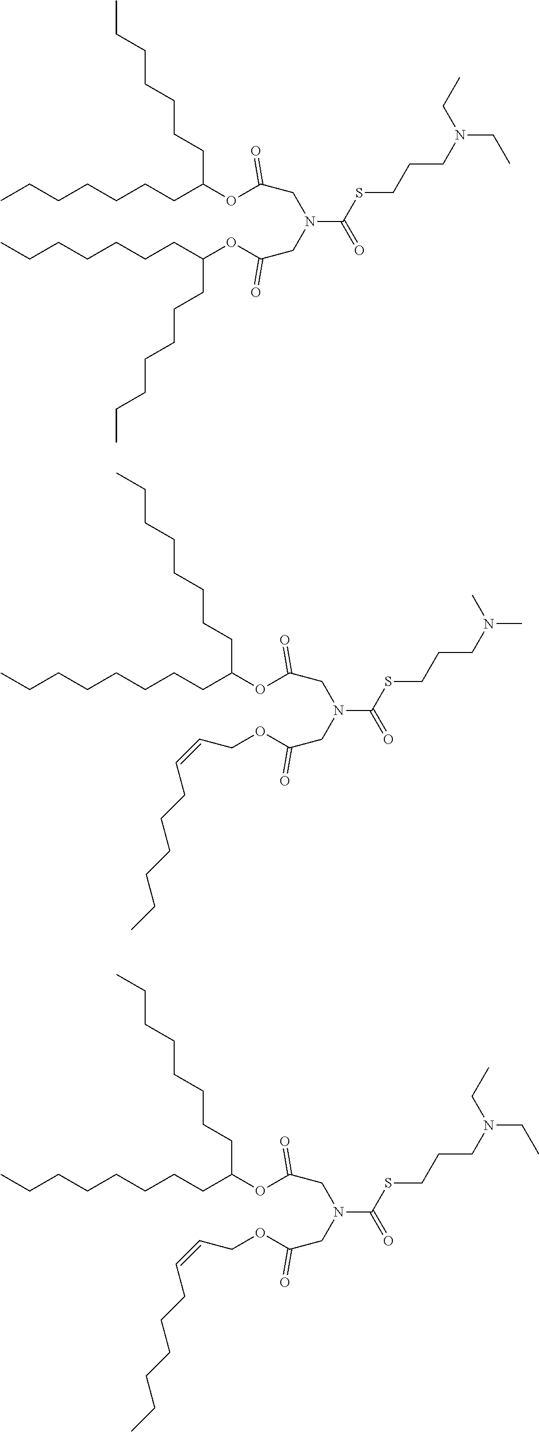

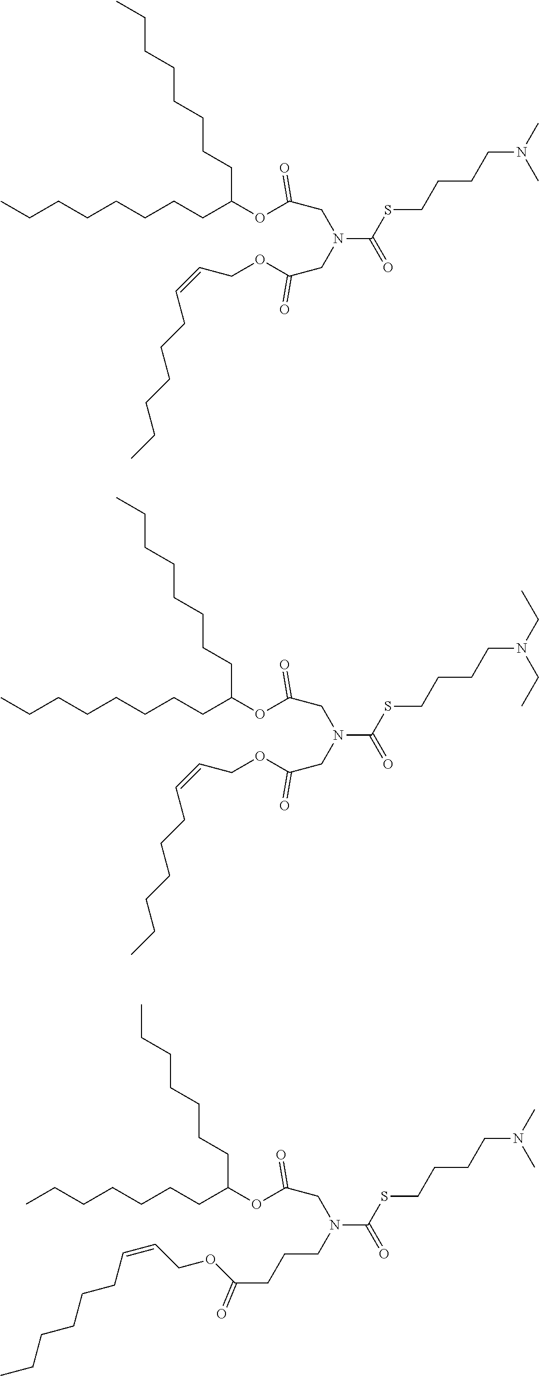

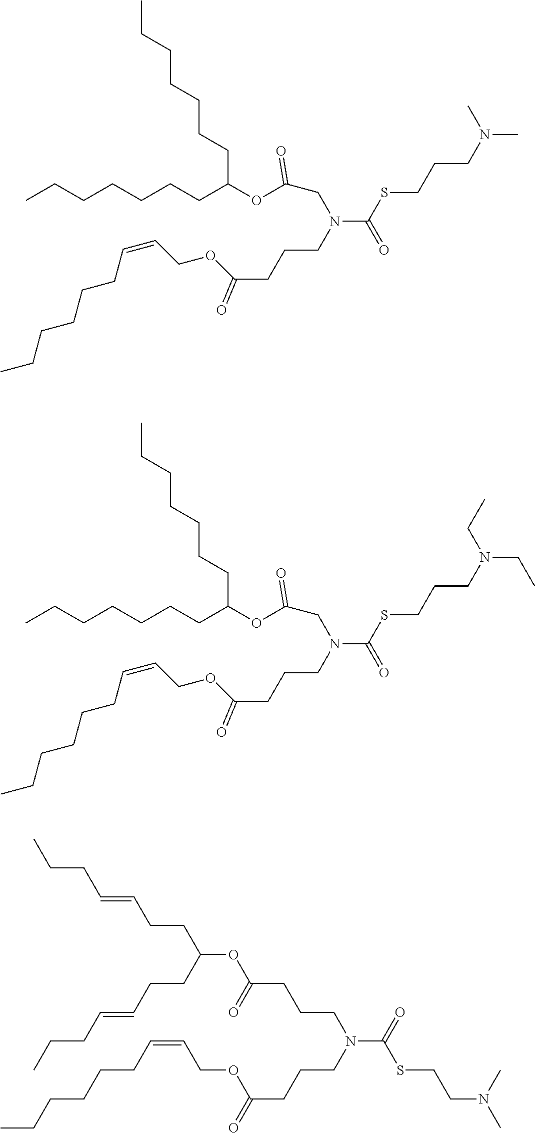

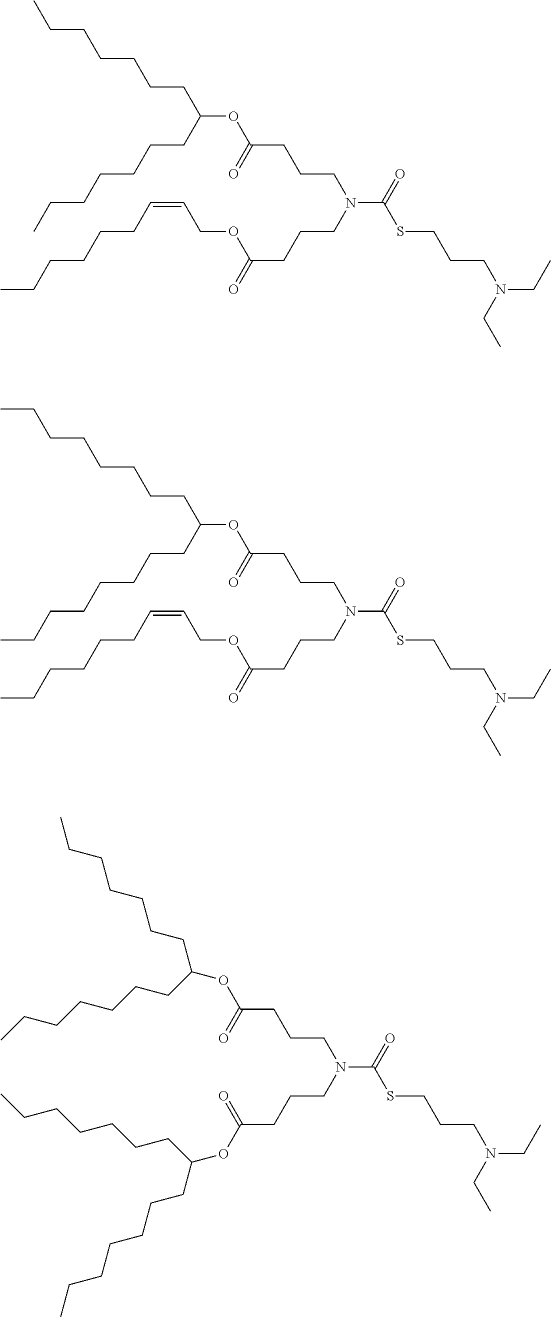

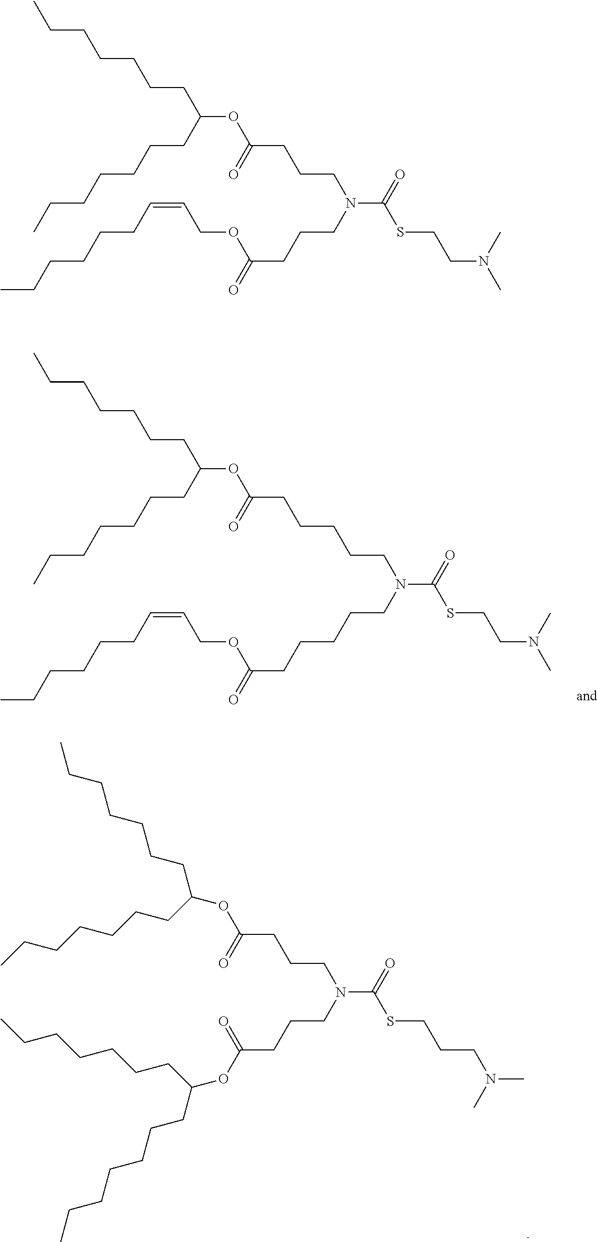

In some embodiments, any one or more lipids recited herein may be expressly excluded.

### Helper Lipids and Sterols

The mRNA-lipid formulations of the present disclosure can comprise a helper lipid, which can be referred to as a neutral lipid, a neutral helper lipid, non-cationic lipid, non-cationic helper lipid, anionic lipid, anionic helper lipid, or a zwitterionic lipid. It has been found that lipid formulations, particularly cationic liposomes and lipid nanoparticles have increased cellular uptake if helper lipids are present in the formulation. (Curr. Drug Metab. 2014; 15(9):882-92). For example, some studies have indicated that neutral and zwitterionic lipids such as 1,2-dioleoyl-sn-glycero-3-phosphatidylcholine (DOPC), Di-Oleoyl-Phosphatidyl-Ethanoalamine (DOPE) and 1,2-DiStearoyl-sn-glycero-3-PhosphoCholine (DSPC), being more fusogenic (i.e., facilitating fusion) than cationic lipids, can affect the polymorphic features of lipid-nucleic acid complexes, promoting the transition from a lamellar to a hexagonal phase, and thus inducing fusion and a disruption of the cellular membrane. (Nanomedicine (Lond). 2014 January; 9(1):105-20). In addition, the use of helper lipids can help to reduce any potential detrimental effects from using many prevalent cationic lipids such as toxicity and immunogenicity.

Non-limiting examples of non-cationic lipids suitable for lipid formulations of the present disclosure include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl.

Additional examples of non-cationic lipids include sterols such as cholesterol and derivatives thereof. One study concluded that as a helper lipid, cholesterol increases the spacing of the charges of the lipid layer interfacing with the nucleic acid making the charge distribution match that of the nucleic acid more closely. (J. R. Soc. Interface. 2012 Mar. 7; 9(68): 548-561). Non-limiting examples of cholesterol derivatives include polar analogues such as 5α-cholestanol, 5α-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5α-cholestanone, and cholesteryl decanoate; and mixtures thereof. In preferred embodiments, the cholesterol derivative is a polar analogue such as cholesteryl-(4′-hydroxy)-butyl ether.

In some embodiments, the helper lipid present in the lipid formulation comprises or consists of a mixture of one or more phospholipids and cholesterol or a derivative thereof. In other embodiments, the helper lipid present in the lipid formulation comprises or consists of one or more phospholipids, e.g, a cholesterol-free lipid formulation. In yet other embodiments, the helper lipid present in the lipid formulation comprises or consists of cholesterol or a derivative thereof, e.g, a phospholipid-free lipid formulation.

Other examples of helper lipids include nonphosphorous containing lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

In some embodiments, the helper lipid comprises from about 20 mol % to about 50 mol %, from about 22 mol % to about 48 mol %, from about 24 mol % to about 46 mol %, about 25 mol % to about 44 mol %, from about 26 mol % to about 42 mol %, from about 27 mol % to about 41 mol %, from about 28 mol % to about 40 mol %, or about 29 mol %, about 30 mol %, about 31 mol %, about 32 mol %, about 33 mol %, about 34 mol %, about 35 mol %, about 36 mol %, about 37 mol %, about 38 mol %, or about 39 mol % (or any fraction thereof or the range therein) of the total lipid present in the lipid formulation.

In some embodiments, the total of helper lipid in the formulation comprises two or more helper lipids and the total amount of helper lipid comprises from about 20 mol % to about 50 mol %, from about 22 mol % to about 48 mol %, from about 24 mol % to about 46 mol %, about 25 mol % to about 44 mol %, from about 26 mol % to about 42 mol %, from about 27 mol % to about 41 mol %, from about 28 mol % to about 40 mol %, or about 29 mol %, about 30 mol %, about 31 mol %, about 32 mol %, about 33 mol %, about 34 mol %, about 35 mol %, about 36 mol %, about 37 mol %, about 38 mol %, or about 39 mol % (or any fraction thereof or the range therein) of the total lipid present in the lipid formulation. In some embodiments, the helper lipids are a combination of DSPC and DOTAP. In some embodiments, the helper lipids are a combination of DSPC and DOTMA.

The cholesterol or cholesterol derivative in the lipid formulation may comprise up to about 40 mol %, about 45 mol %, about 50 mol %, about 55 mol %, or about 60 mol % of the total lipid present in the lipid formulation. In some embodiments, the cholesterol or cholesterol derivative comprises about 15 mol % to about 45 mol %, about 20 mol % to about 40 mol %, about 30 mol % to about 40 mol %, or about 35 mol %, about 36 mol %, about 37 mol %, about 38 mol %, about 39 mol %, or about 40 mol % of the total lipid present in the lipid formulation.

The percentage of helper lipid present in the lipid formulation is a target amount, and the actual amount of helper lipid present in the formulation may vary, for example, by f 5 mol %.

A lipid formulation containing a cationic lipid compound or ionizable cationic lipid compound may be on a molar basis about 20-40% cationic lipid compound, about 25-40% cholesterol, about 25-50% helper lipid, and about 0.5-5% of a polyethylene glycol (PEG) lipid, wherein the percent is of the total lipid present in the formulation. In some embodiments, the composition is about 22-30% cationic lipid compound, about 30-40% cholesterol, about 30-40% helper lipid, and about 0.5-3% of a PEG-lipid, wherein the percent is of the total lipid present in the formulation.

### Lipid Conjugates

The lipid formulations described herein may further comprise a lipid conjugate. The conjugated lipid is useful for preventing the aggregation of particles. Suitable conjugated lipids include, but are not limited to, PEG-lipid conjugates, cationic-polymer-lipid conjugates, and mixtures thereof. Furthermore, lipid delivery vehicles can be used for specific targeting by attaching ligands (e.g., antibodies, peptides, and carbohydrates) to its surface or to the terminal end of the attached PEG chains (Front. Pharmacol. 2015 Dec. 1; 6:286).

In a preferred embodiment, the lipid conjugate is a PEG-lipid. The inclusion of polyethylene glycol (PEG) in a lipid formulation as a coating or surface ligand, a technique referred to as PEGylation, helps protect nanoparticles from the immune system and their escape from RES uptake (Nanomedicine (Lond). 2011 June; 6(4):715-28). PEGylation has been widely used to stabilize lipid formulations and their payloads through physical, chemical, and biological mechanisms. Detergent-like PEG lipids (e.g., PEG-DSPE) can enter the lipid formulation to form a hydrated layer and steric barrier on the surface. Based on the degree of PEGylation, the surface layer can be generally divided into two types, brush-like and mushroom-like layers. For PEG-DSPE-stabilized formulations, PEG will take on the mushroom conformation at a low degree of PEGylation (usually less than 5 mol %) and will shift to brush conformation as the content of PEG-DSPE is increased past a certain level (J. Nanomaterials. 2011; 2011:12). It has been shown that increased PEGylation leads to a significant increase in the circulation half-life of lipid formulations (Annu Rev. Biomed. Eng. 2011 Aug. 15; 13:507-30; J. Control Release. 2010 Aug. 3; 145(3):178-81).

Suitable examples of PEG-lipids include, but are not limited to, PEG coupled to dialkyloxypropyls (PEG-DAA), PEG coupled to diacylglycerol (PEG-DAG), PEG coupled to phospholipids such as phosphatidylethanolamine (PEG-PE), PEG conjugated to ceramides, PEG conjugated to cholesterol or a derivative thereof, and mixtures thereof.

PEG is a linear, water-soluble polymer of ethylene PEG repeating units with two terminal hydroxyl groups. PEGs are classified by their molecular weights and include the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH₂), monomethoxypolyethylene glycol-tresylate (MePEG-TRES), monomethoxypolyethylene glycol-imidazolyl-carbonyl (MePEG-IM), as well as such compounds containing a terminal hydroxyl group instead of a terminal methoxy group (e.g., HO-PEG-S, HO-PEG-S—NHS, HO-PEG-NH₂).

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons). In preferred embodiments, the PEG moiety has an average molecular weight of about 2,000 daltons or about 750 daltons. The average molecular weight may be any value or subvalue within the recited ranges, including endpoints.

In certain instances, the PEG monomers can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester-containing linker moieties and ester-containing linker moieties. In a preferred embodiment, the linker moiety is a non-ester-containing linker moiety. Suitable non-ester-containing linker moieties include, but are not limited to, amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH₂CH₂C(O)—), succinamidyl (—NHC(O)CH₂CH₂C(O)NH—), ether, as well as combinations thereof (such as a linker containing both a carbamate linker moiety and an amido linker moiety). In a preferred embodiment, a carbamate linker is used to couple the PEG to the lipid.

In other embodiments, an ester-containing linker moiety is used to couple the PEG to the lipid. Suitable ester-containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form the lipid conjugate. Such phosphatidylethanolamines are commercially available or can be isolated or synthesized using conventional techniques known to those of skill in the art. Phosphatidylethanolamines containing saturated or unsaturated fatty acids with carbon chain lengths in the range of C₁₀ to C₂₀ are preferred. Phosphatidylethanolamines with mono- or di-unsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoyl-phosphatidylethanolamine (DOPE), and distearoyl-phosphatidylethanolamine (DSPE).

In some embodiments, the PEG-DAA conjugate is a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, or a PEG-distearyloxypropyl (C₁₈) conjugate. In these embodiments, the PEG preferably has an average molecular weight of about 750 to about 2,000 daltons. In particular embodiments, the terminal hydroxyl group of the PEG is substituted with a methyl group.

In addition to the foregoing, other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl, methacrylamide, polymethacrylamide, and polydimethylacrylamide, polylactic acid, polyglycolic acid, and derivatized celluloses such as hydroxymethylcellulose or hydroxyethylcellulose.

In some embodiments, the lipid conjugate (e.g., PEG-lipid) comprises from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 0.9 mol % to about 1.6 mol %, from about 0.9 mol % to about 1.8 mol %, from about 1 mol % to about 1.8 mol %, from about 1 mol % to about 1.7 mol %, from about 1.2 mol % to about 1.8 mol %, from about 1.2 mol % to about 1.7 mol %, from about 1.3 mol % to about 1.6 mol %, or from about 1.4 mol % to about 1.6 mol % (or any fraction thereof or range therein) of the total lipid present in the lipid formulation. In other embodiments, the lipid conjugate (e.g., PEG-lipid) comprises about 0.5%, 0.6%, 0.7%, 0.8%, 0.9%, 1.0%, 1.2%, 1.3%, 1.4%, 1.5%, 1.6%, 1.7%, 1.8%, 1.9%, 2.0%, 2.5%, 3.0%, 3.5%, 4.0%, 4.5%, or 5%, (or any fraction thereof or range therein) of the total lipid present in the lipid formulation. The amount may be any value or subvalue within the recited ranges, including endpoints.

In some preferred embodiments, the PEG-lipid is PEG550-PE. In some preferred embodiments, the PEG-lipid is PEG750-PE. In some preferred embodiments, the PEG-lipid is PEG2000-DMG.

The percentage of lipid conjugate (e.g., PEG-lipid) present in the lipid formulations of the disclosure is a target amount, and the actual amount of lipid conjugate present in the formulation may vary, for example, by ±0.5 mol %. One of ordinary skill in the art will appreciate that the concentration of the lipid conjugate can be varied depending on the lipid conjugate employed and the rate at which the lipid formulation is to become fusogenic.

### Mechanism of Action for Cellular Uptake of Lipid Formulations

Lipid formulations for the intracellular delivery of nucleic acids, particularly liposomes, cationic liposomes, and lipid nanoparticles, are designed for cellular uptake by penetrating target cells through exploitation of the target cells' endocytic mechanisms where the contents of the lipid delivery vehicle are delivered to the cytosol of the target cell. (Nucleic Acid Therapeutics, 28(3):146-157, 2018). Specifically, in the case of a mRNA-lipid formulation targeting hepatocytes described herein, the mRNA-lipid formulation enters hepatocytes through receptor mediated endocytosis. Prior to endocytosis, functionalized ligands such as PEG-lipid at the surface of the lipid delivery vehicle are shed from the surface, which triggers internalization into the target cell. During endocytosis, some part of the plasma membrane of the cell surrounds the vector and engulfs it into a vesicle that then pinches off from the cell membrane, enters the cytosol and ultimately undergoes the endolysosomal pathway. For ionizable cationic lipid-containing delivery vehicles, the increased acidity as the endosome ages results in a vehicle with a strong positive charge on the surface. Interactions between the delivery vehicle and the endosomal membrane then result in a membrane fusion event that leads to cytosolic delivery of the payload. For mRNA payloads, the cell's own internal translation processes will then translate the mRNA or combination of mRNA molecules into the encoded protein (e.g. HBV TALENs). The encoded protein can further undergo post-translational processing, including transportation to a targeted organelle or location within the cell. In the case of the HBV TALENs described herein, the HBV TALEN protein is translocated into the nucleus where the HBV genome may be edited out.

By controlling the composition and concentration of the lipid conjugate, one can control the rate at which the lipid conjugate exchanges out of the lipid formulation and, in turn, the rate at which the lipid formulation becomes fusogenic. In addition, other variables including, e.g., pH, temperature, or ionic strength, can be used to vary and/or control the rate at which the lipid formulation becomes fusogenic. Other methods which can be used to control the rate at which the lipid formulation becomes fusogenic will become apparent to those of skill in the art upon reading this disclosure. Also, by controlling the composition and concentration of the lipid conjugate, one can control the liposomal or lipid particle size.

### Lipid Formulation Manufacture

There are many different methods for the preparation of lipid formulations comprising a nucleic acid, e.g. mRNA or combination of mRNA molecules. (Curr. Drug Metabol. 2014, 15, 882-892; Chem. Phys. Lipids 2014, 177, 8-18; Int. J. Pharm. Stud. Res. 2012, 3, 14-20). The techniques of thin film hydration, double emulsion, reverse phase evaporation, microfluidic preparation, dual asymmetric centrifugation, ethanol injection, detergent dialysis, spontaneous vesicle formation by ethanol dilution, and encapsulation in preformed liposomes are briefly described herein.

### Thin Film Hydration

In Thin Film Hydration (TFH) or the Bangham method, the lipids are dissolved in an organic solvent, then evaporated through the use of a rotary evaporator leading to a thin lipid layer formation. After the layer hydration by an aqueous buffer solution containing the compound to be loaded, Multilamellar Vesicles (MLVs) are formed, which can be reduced in size to produce Small or Large Unilamellar vesicles (LUV and SUV) by extrusion through membranes or by the sonication of the starting MLV.

### Double Emulsion

Lipid formulations can also be prepared through the Double Emulsion technique, which involves lipids dissolution in a water/organic solvent mixture. The organic solution, containing water droplets, is mixed with an excess of aqueous medium, leading to a water-in-oil-in-water (W/O/W) double emulsion formation. After mechanical vigorous shaking, part of the water droplets collapse, giving Large Unilamellar Vesicles (LUVs).

### Reverse Phase Evaporation

The Reverse Phase Evaporation (REV) method also allows one to achieve LUVs loaded with nucleic acid. In this technique a two-phase system is formed by phospholipids dissolution in organic solvents and aqueous buffer. The resulting suspension is then sonicated briefly until the mixture becomes a clear one-phase dispersion. The lipid formulation is achieved after the organic solvent evaporation under reduced pressure. This technique has been used to encapsulate different large and small hydrophilic molecules including nucleic acids.

### Microfluidic Preparation

The Microfluidic method, unlike other bulk techniques, gives the possibility of controlling the lipid hydration process. The method can be classified in continuous-flow microfluidic and droplet-based microfluidic, according to the way in which the flow is manipulated. In the microfluidic hydrodynamic focusing (MHF) method, which operates in a continuous flow mode, lipids are dissolved in isopropyl alcohol which is hydrodynamically focused in a microchannel cross junction between two aqueous buffer streams. Vesicles size can be controlled by modulating the flow rates, thus controlling the lipids solution/buffer dilution process. The method can be used for producing oligonucleotide (ON) lipid formulations by using a microfluidic device consisting of three-inlet and one-outlet ports.

### Dual Asymmetric Centrifugation

Dual Asymmetric Centrifugation (DAC) differs from more common centrifugation as it uses an additional rotation around its own vertical axis. An efficient homogenization is achieved due to the two overlaying movements generated: the sample is pushed outwards, as in a normal centrifuge, and then it is pushed towards the center of the vial due to the additional rotation. By mixing lipids and an NaCl-solution a viscous vesicular phospholipid gel (VPC) is achieved, which is then diluted to obtain a lipid formulation dispersion. The lipid formulation size can be regulated by optimizing DAC speed, lipid concentration and homogenization time.

### Ethanol Injection

The Ethanol Injection (EI) method can be used for nucleic acid encapsulation. This method provides the rapid injection of an ethanolic solution, in which lipids are dissolved, into an aqueous medium containing nucleic acids to be encapsulated, through the use of a needle. Vesicles are spontaneously formed when the phospholipids are dispersed throughout the medium.

### Detergent Dialysis

The Detergent dialysis method can be used to encapsulate nucleic acids. Briefly lipid and plasmid are solubilized in a detergent solution of appropriate ionic strength, after removing the detergent by dialysis, a stabilized lipid formulation is formed. Unencapsulated nucleic acid is then removed by ion-exchange chromatography and empty vesicles by sucrose density gradient centrifugation. The technique is highly sensitive to the cationic lipid content and to the salt concentration of the dialysis buffer, and the method is also difficult to scale.

### Spontaneous Vesicle Formation by Ethanol Dilution

Stable lipid formulations can also be produced through the Spontaneous Vesicle Formation by Ethanol Dilution method in which a stepwise or dropwise ethanol dilution provides the instantaneous formation of vesicles loaded with nucleic acid by the controlled addition of lipid dissolved in ethanol to a rapidly mixing aqueous buffer containing the nucleic acid.

### Encapsulation in Preformed Liposomes

The entrapment of nucleic acids can also be obtained starting with preformed liposomes through two different methods: (1) a simple mixing of cationic liposomes with nucleic acids which gives electrostatic complexes called “lipoplexes”, where they can be successfully used to transfect cell cultures, but are characterized by their low encapsulation efficiency and poor performance in vivo; and (2) a liposomal destabilization, slowly adding absolute ethanol to a suspension of cationic vesicles up to a concentration of 40% v/v followed by the dropwise addition of nucleic acids achieving loaded vesicles; however, the two main steps characterizing the encapsulation process are too sensitive, and the particles have to be downsized.

In certain embodiments, examples of lipids and lipid nanoparticles, pharmaceutical compositions comprising the lipids, methods of making the lipids or formulating pharmaceutical compositions comprising the lipids and nucleic acid molecules, and methods of using the pharmaceutical compositions for treating or preventing diseases are described in U.S. or International Patent Application Publications, such as US2017/0190661, US2006/0008910, US2015/0064242, US2005/0064595, WO/2019/036030, US2019/0022247, WO/2019/036028, WO/2019/036008, WO/2019/036000, US2016/0376224, US2017/0119904, WO/2018/200943, WO/2018/191657, WO/2018/118102, US20180169268, WO2018118102, WO2018119163, US2014/0255472, and US2013/0195968, the relevant content of each of which is hereby incorporated by reference in its entirety.

### Cells and Polypeptides

The application also provides cells, preferably isolated cells, comprising any of the polynucleotides and vectors described herein. The cells can, for instance, be used for recombinant protein production, or for the production of viral particles. In some embodiments, the cells can be used for production of HBV TALENs. The term “isolated” as used herein, refers to a cell that is removed from an organism in which it was originally found, or a descendant of such a cell. In some embodiments, the cells comprising a polynucleotide or vector described herein can be in or part of a subject, instead of being isolated. In these embodiments, the cells of a subject may be used for in vivo recombinant protein production or production of viral particles. In some embodiments, the cells are cultured in vitro, for example in the presence of other cells. In some embodiments, the cell is later introduced into a second organism or reintroduced into the organism from which it (or the cell from which it is descended) was isolated.

Host cells comprising an HBV TALEN or a nucleic acid encoding an HBV TALEN of the application also form part of the invention. The HBV TALENs may be produced through recombinant DNA technology involving expression of the molecules in host cells, e.g. Chinese hamster ovary (CHO) cells, tumor cell lines, BHK cells, human cell lines such as HEK293 cells, PER.C6 cells, or yeast, bacteria, fungi, insect cells, and the like, or transgenic animals or plants. In certain embodiments, the cells are from a multicellular organism, in certain embodiments they are of vertebrate or invertebrate origin. In certain embodiments, the cells are mammalian cells, such as human cells, or insect cells. In certain embodiments, the cells are primary cells, e.g., liver cells, more specifically infected liver cells, HBV-infected liver cells, or liver cells harboring HBV cccDNA. In general, the production of a recombinant protein, such the HBV TALENs of the invention, in a host cell comprises the introduction of a heterologous nucleic acid molecule encoding the protein in expressible format into the host cell, culturing the cells under conditions conducive to expression of the nucleic acid molecule and allowing expression of the protein in said cell. The nucleic acid molecule encoding a protein in expressible format may be in the form of an expression cassette, and usually requires sequences capable of bringing about expression of the nucleic acid, such as enhancer(s), promoter, polyadenylation signal, and the like. The person skilled in the art is aware that various promoters can be used to obtain expression of a gene in host cells. Promoters can be constitutive or regulated, and can be obtained from various sources, including viruses, prokaryotic, or eukaryotic sources, or artificially designed. Further regulatory sequences may be added. Many promoters can be used for expression of a transgene(s), and are known to the skilled person, e.g. these may comprise viral, mammalian, synthetic promoters, and the like. A non-limiting example of a suitable promoter for obtaining expression in eukaryotic cells is a CMV-promoter (U.S. Pat. No. 5,385,839), e.g. the CMV immediate early promoter, for instance comprising nt. −735 to +95 from the CMV immediate early gene enhancer/promoter. A polyadenylation signal, for example the bovine growth hormone polyA signal (U.S. Pat. No. 5,122,458), may be present behind the transgene(s). Alternatively, several widely used expression vectors are available in the art and from commercial sources, e.g. the pcDNA and pEF vector series of Invitrogen, pMSCV and pTK-Hyg from BD Sciences, pCMV-Script from Stratagene, etc, which can be used to recombinantly express the protein of interest, or to obtain suitable promoters and/or transcription terminator sequences, polyA sequences, and the like.

The cell culture can be any type of cell culture, including adherent cell culture, e.g. cells attached to the surface of a culture vessel or to microcarriers, as well as suspension culture. Most large-scale suspension cultures are operated as batch or fed-batch processes because they are the most straightforward to operate and scale up. Nowadays, continuous processes based on perfusion principles are becoming more common and are also suitable. Suitable culture media are also well known to the skilled person and can generally be obtained from commercial sources in large quantities, or custom-made according to standard protocols. Culturing can be done for instance in dishes, roller bottles or in bioreactors, using batch, fed-batch, continuous systems and the like. Suitable conditions for culturing cells are known (see e.g. Tissue Culture, Academic Press, Kruse and Paterson, editors (1973), and R. I. Freshney, Culture of animal cells: A manual of basic technique, fourth edition (Wiley-Liss Inc., 2000, ISBN 0-471-34889-9)). Cell culture media are available from various vendors, and a suitable medium can be routinely chosen for a host cell to express the protein of interest, here the HBV TALENs. The suitable medium may or may not contain serum.

Embodiments of the application thus also relate to a method of making an HBV TALEN of the application. The method comprises transfecting a host cell with an expression vector comprising a polynucleotide encoding an HBV TALEN of the application operably linked to a promoter, growing the transfected cell under conditions suitable for expression of the HBV TALEN, and optionally purifying or isolating the HBV TALEN expressed in the cell. The HBV TALEN can be isolated or collected from the cell by any method known in the art including affinity chromatography, size exclusion chromatography, etc. Techniques used for recombinant protein expression will be well known to one of ordinary skill in the art in view of the present disclosure. The expressed HBV TALEN can also be studied without purifying or isolating the expressed protein, e.g., by analyzing the supernatant of cells transfected with an expression vector encoding the HBV TALEN and grown under conditions suitable for expression of the HBV TALEN.

Thus, also provided are non-naturally occurring or recombinant polypeptides comprising an amino acid sequence that is at least 90% identical to the amino acid sequence of one or more of SEQ ID NOs: 25, 26, 27 or 28. In some embodiments, a non-naturally occurring or recombinant polypeptide with a TALE DNA binding domain comprises the amino acid sequence of one or more of SEQ ID NO: 25, 26, 27 or 28. In some embodiments, a combination of non-naturally occurring or recombinant polypeptides with TALE DNA binding domains comprises the amino acid sequences of SEQ ID NO: 25 and SEQ ID NO: 26. In some embodiments, a combination of non-naturally occurring or recombinant polypeptides with TALE DNA binding domains comprises the amino acid sequences of SEQ ID NO: 27 and SEQ ID NO: 28. As described above and below, isolated nucleic acid molecules encoding these sequences, vectors comprising these sequences operably linked to a promoter, and compositions comprising the polypeptide, polynucleotide, or vector are also contemplated by the application.

In an embodiment of the application, a recombinant polypeptide comprises an amino acid sequence that is at least 90% identical to the amino acid sequence of one or more of SEQ ID NO: 25, 26, 27 or 28, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to one or more of SEQ ID NO: 25, 26, 27 or 28, respectively. In some embodiments, a combination of recombinant polypeptides comprises amino acid sequences that are at least 90% identical to the amino acid sequence of SEQ ID NO: 25 and 26, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 25 and 26, respectively. In some embodiments, a combination of recombinant polypeptides comprises amino acid sequences that are at least 90% identical to the amino acid sequence of SEQ ID NO: 27 and 28, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 27 and 28, respectively. Preferably, a non-naturally occurring or recombinant polypeptide consists of one or more of SEQ ID NO: 25, 26, 27 or 28. Preferably, a combination of non-naturally occurring or recombinant polypeptides consists of SEQ ID NOs: 25 and 26, or SEQ ID NOs: 27 and 28.

### Compositions

The application also relates to compositions, more particularly pharmaceutical compositions, comprising one or more HBV TALENs, combinations, polynucleotides (including RNA and DNA, preferably mRNA) encoding one more HBV TALENs, vectors, LNPs and/or host cells according to the application. Any of the HBV TALENs, combinations, nucleic acids, vectors, LNPs and/or host cells of the application described herein can be used in the compositions or pharmaceutical compositions of the application.

The application provides, for example, a pharmaceutical composition comprising any nucleic acid molecule, vector, combination, LNP or host cell described herein, together with a pharmaceutically acceptable carrier. A pharmaceutically acceptable carrier is non-toxic and should not interfere with the efficacy of the active ingredient. Pharmaceutically acceptable carriers can include one or more excipients such as binders, disintegrants, swelling agents, suspending agents, emulsifying agents, wetting agents, lubricants, flavorants, sweeteners, preservatives, dyes, solubilizers and coatings. The precise nature of the carrier or other material can depend on the route of administration, e.g., intramuscular, intradermal, subcutaneous, oral, intravenous, cutaneous, intramucosal (e.g., gut), intranasal or intraperitoneal routes. For liquid injectable preparations, for example, suspensions and solutions, suitable carriers and additives include water, glycols, oils, alcohols, preservatives, coloring agents and the like. For solid oral preparations, for example, powders, capsules, caplets, gelcaps and tablets, suitable carriers and additives include starches, sugars, diluents, granulating agents, lubricants, binders, disintegrating agents and the like. For nasal sprays/inhalant mixtures, the aqueous solution/suspension can comprise water, glycols, oils, emollients, stabilizers, wetting agents, preservatives, aromatics, flavors, and the like as suitable carriers and additives.

Pharmaceutical compositions of the application can be formulated in any matter suitable for administration to a subject to facilitate administration and improve efficacy, including, but not limited to, oral (enteral) administration and parenteral injections. The parenteral injections include intravenous injection or infusion, subcutaneous injection, intradermal injection, and intramuscular injection. Pharmaceutical compositions of the application can also be formulated for other routes of administration including transmucosal, ocular, rectal, long acting implantation, sublingual administration, under the tongue, from oral mucosa bypassing the portal circulation, inhalation, or intranasal.

In a preferred embodiment of the application, pharmaceutical compositions of the application are formulated for parental injection, preferably subcutaneous, intradermal injection, or intramuscular injection, more preferably intramuscular injection.

According to embodiments of the application, pharmaceutical compositions for administration will typically comprise a buffered solution in a pharmaceutically acceptable carrier, e.g., an aqueous carrier such as buffered saline and the like, e.g., phosphate buffered saline (PBS). The compositions can also contain pharmaceutically acceptable substances as required to approximate physiological conditions such as pH adjusting and buffering agents. For example, a pharmaceutical composition of the application comprising plasmid DNA can contain phosphate buffered saline (PBS) as the pharmaceutically acceptable carrier. The plasmid DNA can be present in a concentration of, e.g., 0.5 mg/mL to 5 mg/mL, such as 0.5 mg/mL, 1 mg/mL, 2 mg/mL, 3 mg/mL, 4 mg/mL, or 5 mg/mL, preferably at 1 mg/mL.

In some embodiments, a pharmaceutical composition of the application comprising a lipid nanoparticle can be administered in a concentration of, e.g., about 20 μg/mL to about 200 μg/mL, such as 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, or 200 μg/mL. In some embodiments, a pharmaceutical composition of the application comprising a lipid nanoparticle can be administered in a concentration below 20 μg/mL. In some embodiments, a pharmaceutical composition of the application comprising a lipid nanoparticle can be administered in a concentration above 200 μg/mL.

The application also provides methods of making pharmaceutical compositions of the application. A method of producing a pharmaceutical composition comprises mixing an isolated polynucleotide encoding an HBV TALEN, vector, and/or LNP of the application with one or more pharmaceutically acceptable carriers. One of ordinary skill in the art will be familiar with conventional techniques used to prepare such compositions.

### Methods of Treatment

The application provides methods for treating hepatitis. In some embodiments, the method is for treatment of a hepatitis B virus (HBV), specifically an HBV infection, in a subject in need thereof, comprising administering to the subject an effective amount of a pharmaceutical composition of the application. Some embodiments relate to methods of treating HBV/HDV co-infection. Some embodiments relate to methods of reducing HBV surface antigens. Some embodiments relate to methods of reducing or eradicating HBV cccDNA. Some embodiments relate to methods of targeting integrated HBV DNA. Any of the pharmaceutical compositions of the application described herein can be used in the methods of the application. The application also provides methods for reducing infection and/or replication of HBV in a subject, comprising administering to the subject a pharmaceutical composition of the application. The term “reducing” or “reduced” as used herein with regards to an infection or replication of a virus generally means a statistically significant decreased amount of infection and/or replication as compared to a reference level, such as an untreated subject or a subject administered a control treatment. However, for avoidance of doubt, “reducing” means decreasing by at least 10% as compared to a reference level, for example decreasing by at least about 20%, or at least about 30%, or at least about 40%, or at least about 50%, or at least about 60%, or at least about 70%, or at least about 75%, or at least about 80%, or at least about 90%, or up to and including a 100% decrease (i.e., absent level as compared to a reference level), or any decrease between 10% and 100% as compared to a reference level. The level of infection and/or replication can be ascertained by one of ordinary skill in the art in view of the present disclosure.

The terms “treat”, “treated”, or “treating” as used herein refers to both therapeutic treatment and prophylactic or preventative measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this disclosure, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects. Treatment also includes prolonging survival as compared to expected survival if not receiving treatment.

As used herein, the term “infection” refers to the invasion of a host by a disease-causing agent. A disease-causing agent is considered to be “infectious” when it is capable of invading a host, and replicating or propagating within the host. Examples of infectious agents include viruses, e.g., HBV, HDV and certain species of adenovirus, prions, bacteria, fungi, protozoa and the like. “HBV infection” specifically refers to invasion of a host organism, such as cells and tissues of the host organism, by HBV. In some embodiments, the infection is a co-infection with HBV and HDV.

As used herein, a “therapeutically effective amount” or “effective amount” means an amount of a composition, polynucleotide, or vector sufficient to induce a desired effect or response in a subject in need thereof. An effective amount can be an amount sufficient to provide a therapeutic effect against a disease such as HBV infection. An effective amount can vary depending upon a variety of factors, such as the physical condition of the subject, age, weight, health, etc.; the particular application, e.g., therapeutic treatment; and the particular disease, e.g., viral infection, for which immunity is desired. An effective amount can readily be determined by one of ordinary skill in the art in view of the present disclosure. The therapeutically effective amount can be ascertained experimentally, for example by assaying blood concentration of the compound, or theoretically, by calculating bioavailability by one of ordinary skill in the art in view of the present disclosure.

In particular embodiments of the application, an effective amount refers to the amount of a composition or combination which is sufficient to achieve one, two, three, four, or more of the following effects: (i) reduce or ameliorate the severity of an HBV infection or a symptom associated therewith; (ii) reduce the duration of an HBV infection or symptom associated therewith; (iii) prevent the progression of an HBV infection or symptom associated therewith; (iv) cause regression of an HBV infection or symptom associated therewith; (v) prevent the development or onset of an HBV infection, or symptom associated therewith; (vi) prevent the recurrence of an HBV infection or symptom associated therewith; (vii) reduce hospitalization of a subject having an HBV infection; (viii) reduce hospitalization length of a subject having an HBV infection; (ix) increase the survival of a subject with an HBV infection; (x) eliminate an HBV infection in a subject; (xi) inhibit or reduce HBV replication in a subject; and/or (xii) enhance or improve the prophylactic or therapeutic effect(s) of another therapy.

A therapeutically effective amount can also be an amount of the compound sufficient to reduce HBsAg levels consistent with evolution to clinical seroconversion; and/or to achieve sustained HBsAg clearance associated with reduction of infected hepatocytes by a subject's immune system; induce HBV-antigen specific activated T-cell populations; and/or to achieve loss of detectable HBsAg during or after treatment that then preferably persists at 6 months or more after the end of treatment, most preferably for life. Examples of a target index include serum HBsAg level that is below a threshold of e.g., 100 IU/mL of HBsAg and/or HBV-specific CD8 T cells responses of greater numbers or of greater polyfunctionality than e.g., at the beginning of the treatment. Additional examples of target indexes include, but are not limited to, serum HBV RNA levels lower than the lower limit of quantification (LLoQ); and/or serum ALT concentration lower than 3 times the upper normal limit, or lower than 129 U/L if the subject is a male subject, or lower than 108 U/L if the subject is a female subject, more particularly a serum ALT concentration lower than 120 U/L if the subject is a male subject or lower than 105 U/L if the subject is a female subject, more particularly a serum ALT concentration lower than 90 U/L if the subject is a male subject or lower than 57 U/L if the subject is a female subject; and/or HBeAg-negative serum; and/or serum HBsAg level of 100 IU/mL or lower, more particularly of 10 IU/mL or lower which would be considered normalized; and/or HBs seroconversion; and/or core-related antigen (crAg) below LLoQ. In some preferred embodiments, an effective amount is an amount sufficient for an expression level of one or more of HBsAg, HBeAg, HBV DNA, HBV cccDNA, or integrated HBV DNA to be reduced in the subject. In some embodiments, the expression level is a hepatocyte level, a nuclear or cellular level, a liver level, a serum level, or a plasma level. In some preferred embodiments, an effective amount is an amount sufficient for serum and/or plasma level of one or more of HBsAg, HBeAg, and HBV DNA to be reduced in the subject.

As general guidance, an effective amount when used with reference to a nucleic acid molecule or vector can range from about 0.1 mg/kg of nucleic acid molecule or vector to about 5 mg/kg of nucleic acid molecule or vector, such as about 0.1 mg/kg, about 0.25 mg/kg, about 0.5 mg/kg, about 0.75 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, or about 5.0 mg/kg. Preferably, an effective amount of a nucleic acid molecule or vector is about 0.25 mg/kg to about 3 mg/kg. An effective amount when used with reference to a nucleic acid molecule or vector in a pharmaceutical composition can range from a concentration of about 0.01 mg/mL to about 2 mg/mL of a nucleic acid molecule or vector total, such as 0.01 mg/mL, 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.1 mg/mL, 0.25 mg/mL, 0.5 mg/mL, 0.75 mg/mL, 1 mg/mL, 1.5 mg/mL, or 2 mg/mL. Preferably, an effective amount of a molecule or vector is less than 1 mg/mL, more preferably less than 0.05 mg/mL. An effective amount can be from one nucleic acid molecule or vector or from multiple nucleic acid molecules or vectors. An effective amount can be administered in a single composition, or in multiple compositions, such as 1, 2, 3, 4, 5, 6, 7, 8, 9, or 10 compositions (e.g., tablets, capsules or injectables, or any composition adapted to intradermal delivery, e.g., to intradermal delivery using an intradermal delivery patch), wherein the administration of the multiple capsules or injections collectively provides a subject with an effective amount. For example, when two DNA plasmids are used, an effective amount can be 3-4 mg/mL, with 1.5-2 mg/mL of each plasmid. An effective amount can be administered as a single therapeutically effective dose or in a series of therapeutically effective doses, such as 1, 2, 3, 4, 5, or more doses, or wherein the series of doses collectively provides a subject with a therapeutically effective amount. When multiple doses are administered, each dose can be the same amount and/or concentration, or a different amount and/or concentration. The optimization of dosing strategies will be readily understood and practiced by one of ordinary skill in the art.

A combination comprising two nucleic acid molecules or vectors, e.g., a first vector encoding a first HBV TALEN and second vector encoding a second HBV TALEN, can be administered to a subject by mixing both vectors or nucleic acid molecules and delivering the mixture to a single anatomic site. Alternatively, two separate administrations, each delivering a single vector or nucleic acid molecule, can be performed. In such embodiments, whether both vectors or nucleic acid molecules are administered in a single administration as a mixture or in two separate administrations, the first vector or nucleic acid molecule and the second vector or nucleic acid molecule can be administered in a ratio of 10:1 to 1:10, by weight, such as 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10, by weight. Preferably, the first and second vectors or first and second nucleic acid molecules are administered in a ratio of 1:1, by weight.

Preferably, a subject to be treated according to the methods of the application is a hepatitis-infected subject, preferably an HBV-infected subject, particularly a subject having chronic HBV infection. In some embodiments, a subject is co-infected with HBV and HDV. Acute HBV infection is characterized by an efficient activation of the innate immune system complemented with a subsequent broad adaptive response (e.g., HBV-specific T-cells, neutralizing antibodies), which usually results in successful suppression of replication or removal of infected hepatocytes. In contrast, such responses are impaired or diminished due to high viral and antigen load, e.g., HBV envelope proteins are produced in abundance and can be released in sub-viral particles in 1,000-fold excess to infectious virus.

Chronic HBV infection is described in phases characterized by viral load, liver enzyme levels (necroinflammatory activity), HBeAg, or HBsAg load or presence of antibodies to these antigens. cccDNA levels stay relatively constant at approximately 10 to 50 copies per cell, even though viremia can vary considerably. The persistence of the cccDNA species leads to chronicity. More specifically, the phases of chronic HBV infection include: (i) the immune-tolerant phase characterized by high viral load and normal or minimally elevated liver enzymes; (ii) the immune activation HBeAg-positive phase in which lower or declining levels of viral replication with significantly elevated liver enzymes are observed; (iii) the inactive HBsAg carrier phase, which is a low replicative state with low viral loads and normal liver enzyme levels in the serum that may follow HBeAg seroconversion; and (iv) the HBeAg-negative phase in which viral replication occurs periodically (reactivation) with concomitant fluctuations in liver enzyme levels, mutations in the pre-core and/or basal core promoter are common, such that HBeAg is not produced by the infected cell. Preferably, an effective amount refers to the amount of a composition or combination of the application which is sufficient to treat chronic HBV infection.

In some embodiments, a subject having chronic HBV infection is undergoing nucleoside analog (NUC) treatment, and is NUC-suppressed. As used herein, “NUC-suppressed” refers to a subject having an undetectable viral level of HBV and stable alanine aminotransferase (ALT) levels for at least six months. Examples of nucleoside/nucleotide analog treatment include HBV polymerase inhibitors, such as entecavir and tenofovir. Preferably, a subject having chronic HBV infection does not have advanced hepatic fibrosis or cirrhosis. Such subject would typically have a METAVIR score of less than 3 for fibrosis and a fibroscan result of less than 9 kPa. The METAVIR score is a scoring system that is commonly used to assess the extent of inflammation and fibrosis by histopathological evaluation in a liver biopsy of patients with hepatitis B. The scoring system assigns two standardized numbers: one reflecting the degree of inflammation and one reflecting the degree of fibrosis.

It is believed that elimination or reduction of chronic HBV may allow early disease interception of severe liver disease, including virus-induced cirrhosis and hepatocellular carcinoma. Thus, the methods of the application can also be used as therapy to treat HBV-induced diseases. Examples of HBV-induced diseases include, but are not limited to cirrhosis, cancer (e.g., hepatocellular carcinoma), and fibrosis, particularly advanced fibrosis characterized by a METAVIR score of 3 or higher for fibrosis. In such embodiments, an effective amount is an amount sufficient to achieve persistent loss of HBsAg within 12 months and significant decrease in clinical disease (e.g., cirrhosis, hepatocellular carcinoma, etc.).

The phrase “inducing an immune response” when used with reference to the methods described herein encompasses providing a therapeutic immunity for treating against a pathogenic agent, e.g., HBV. In an embodiment, “inducing an immune response” means producing an immunity in a subject in need thereof, e.g., to provide a therapeutic effect against a disease, such as HBV infection or co-infection with HBV and HDV. In certain embodiments, “inducing an immune response” refers to causing or improving cellular immunity, e.g., HBV-specific CD4+ and CD8+ T cell responses. In certain embodiments, this T cell response can bring about functional cure for the treated patient who has CHB. In certain embodiments, “inducing an immune response” refers to causing or improving a humoral immune response against HBV infection or co-infection with HBV and HDV. In certain embodiments, “inducing an immune response” refers to causing or improving a cellular and a humoral immune response against HBV infection or co-infection with HBV and HDV.

As used herein, the term “functional cure” or “FC” refers to a state of a subject who had CHB where the serum of the subject remains free of detectable HBV DNA and HBsAg after the subject is off all HBV treatment(s) for at least 6 months. For example, the serum of the subject with FC has undetectable HBV DNA and is HBsAg-negative 6 months after the end of HBV treatment. The HBsAg loss in a subject with FC can be with or without HBsAg seroconversion. In some embodiment, the FC lasts at least 1 year, preferably at least 2 years, 3 years, 4 years, 5 years, 6 years, 7 years, 8 years, 9 years or 10 years. Most preferably, the FC lasts for life.

As used herein, the term “recovery of T cell function” refers to the re-activation of exhausted and otherwise non-functional HBV-specific T cells such that they are able to perform their normal effector functions. Examples of recovered HBV-specific T cell response include, but are not limited to, killing or non-cytolytic control of HBV-infected hepatocytes, killing of hepatocytes with integrated HBV DNA expressing HBsAg and, once FC is achieved, performing surveillance and killing infected cells that reactivate over time. The net effect is to keep the serum free of virus products, such as HBV DNA, HBsAg and other HBV proteins.

In some embodiments, the disclosed mRNA molecules can be used in combination with one or more additional agent(s) for treating and/or inhibiting replication Hepatitis infection (e.g., HBV and/or HDV). The additional agent can be one or more immune modulators. Additional agents include, but are not limited to, an interferon, a nucleoside/nucleotide analog, a capsid assembly modulator, a sequence specific oligonucleotide (such as an anti-sense oligonucleotide and/or an siRNA), an entry inhibitor, and/or a therapeutic HBV vaccine. In some embodiments, the disclosed mRNA molecules are administered in combination with a cytokine. In some embodiments, the disclosed mRNA molecules are administered in combination with thymosin alpha-1. In some embodiments, the disclosed mRNA molecules are administered in combination with standard of care treatment for Hepatitis infection. Standard of care treatment for HBV infection can include inhibitors of viral polymerase such as nucleotide/nucleotide analogs (e.g., Lamivudine, Telbivudine, Entecavir, Adefovir, Tenofovir, and Clevudine, Tenofovir alafenamide (TAF), CMX157, and AGX-1009) and Interferons (e.g., Peg-IFN-2a and IFN-a-2b, Interferon lambda, recombinant interferon alpha 2b, IFN-a). In some embodiments, the disclosed mRNA molecules are administered in combination with an interferon selected from the group consisting of interferon alpha-2a; interferon alpha-2b; interferon alpha-N3; interferon beta-1a; interferon beta-1b; interferon gama-1b; interferon lambda-1; interferon lambda-2; interferon lambda-3; pegylated interferon alpha-2a; pegylated interferon alpha-2b; pegylated interferon lambda-1; and pegylated interferon lambda-2. In some embodiments, the disclosed mRNA molecules are administered in combination with one or more TLR7 agonists. In some embodiments, the disclosed mRNA molecules are administered in combination with one or more TLR8 agonists. In some embodiments, the disclosed mRNA molecules are administered in combination with GS-962O (4-amino-2-butoxy-8-3-(pyrrolidin-1-ylmethyl)phenyl)methyl-5,7-dihydropteridin-6-one) or GS-9688 (Selgantolimod). In some embodiments, the disclosed mRNA molecules are administered in combination with Bulevirtide.

In some embodiments, the disclosed mRNA molecules are administered in combination with one or more oligonucleotides after either simultaneous (co-administration) or sequential dosing. Oligonucleotides can include siRNA, ASO, Gapmer, and/or nucleic acid polymer (NAP). In some embodiments, the disclosed mRNA molecules are administered in combination with one or more antiviral agents such as viral replication inhibitors. In some embodiments, the disclosed mRNA molecules are administered in combination with HBV Capsid assembly modulators (CAM). HBV CAM molecules can include JNJ 6379, NVR 3-778, AB-423, GLS-4, Bayer 41-4109, HAP-1, and AT-1. In some embodiments, the disclosed oligonucleotide constructs are administered in combination with one or more immunomodulators such as TLR agonists. TLR agonists can include GS-9620, GS-9688, ARB-1598, ANA975, RG7795(ANA773), MEDI9197, PF-3512676, and IMO-2055. In some embodiments, the disclosed mRNA molecules are administered in combination with HBV vaccines. HBV vaccines can include Heplislav, ABX203, and INO-1800.

In one aspect, the siRNA component comprises one or more siRNA agents. Each siRNA agent disclosed herein includes at least a sense strand and an antisense strand. The sense strand and the antisense strand can be partially, substantially, or fully complementary to each other. The length of the siRNA agent sense and antisense strands described herein each can be 16 to 30 nucleotides in length. In some embodiments, the sense and antisense strands are independently 17 to 26 nucleotides in length. In some embodiments, the sense and antisense strands are independently 19 to 26 nucleotides in length. In some embodiments, the sense and antisense strands are independently 21 to 26 nucleotides in length. In some embodiments, the sense and antisense strands are independently 21 to 24 nucleotides in length. The sense and antisense strands can be either the same length or different lengths. The HBV siRNA agents disclosed herein have been designed to include antisense strand sequences that are at least partially complementary to a sequence in the HBV genome that is conserved across the majority of known serotypes of HBV. The siRNA agents described herein, upon delivery to a cell expressing HBV, inhibit the expression of one or more HBV genes in vivo or in vitro. In some embodiments, the siRNA molecules can be JNJ 3989, VIR-2218; or ARB-1467.

A siRNA agent includes a sense strand (also referred to as a passenger strand) that includes a first sequence, and an antisense strand (also referred to as a guide strand) that includes a second sequence. A sense strand of the HBV siRNA agents described herein includes a core stretch having at least about 85% identity to a nucleotide sequence of at least 16 consecutive nucleotides in an HBV mRNA. In some embodiments, the sense strand core nucleotide stretch having at least about 85% identity to a sequence in an HBV mRNA is 16, 17, 18, 19, 20, 21, 22, or 23 nucleotides in length. An antisense strand of an HBV siRNA agent comprises a nucleotide sequence having at least about 85% complementary over a core stretch of at least 16 consecutive nucleotides to a sequence in an HBV mRNA and the corresponding sense strand. In some embodiments, the antisense strand core nucleotide sequence having at least about 85% complementarity to a sequence in an HBV mRNA or the corresponding sense strand is 16, 17, 18, 19, 20, 21, 22, or 23 nucleotides in length.

Methods according to embodiments of the application further comprise administering to the subject in need thereof another anti-HBV agent (such as a nucleoside analog or other anti-HBV agent) in combination with a pharmaceutical composition of the application. For example, another anti-HBV agent can be a small molecule or antibody, including, but not limited to, immune checkpoint inhibitors (e.g., anti-PD1, anti-TIM-3, etc.), toll-like receptor agonists (e.g., TLR7 agonists and/or TLR8 agonists), RIG-1 agonists, IL-15 superagonists (Altor Bioscience), mutant IRF3 and IRF7 genetic adjuvants, STING agonists (Aduro), FLT3L genetic adjuvant, IL-12 genetic adjuvant, IL-7-hyFc; CAR-T which bind HBV env (S-CAR cells); capsid assembly modulators; cccDNA inhibitors, HBV polymerase inhibitors (e.g., entecavir and tenofovir). The one or other anti-HBV active agents can be, for example, a small molecule, an antibody or antigen binding fragment thereof, a polypeptide, protein, or nucleic acid.

### Methods of Delivery

Pharmaceutical compositions and combinations of the application can be administered to a subject by any method known in the art in view of the present disclosure, including, but not limited to, parenteral administration (e.g., intramuscular, subcutaneous, intravenous, or intradermal injection), oral administration, transdermal administration, and nasal administration. Preferably, pharmaceutical compositions and combinations are administered parenterally (e.g., by intravenous injection, intramuscular injection or intradermal injection) or transdermally.

In some embodiments of the application in which a pharmaceutical composition or combination comprises one or more DNA plasmids, administration can be by injection through the skin, e.g., intramuscular or intradermal injection, preferably intramuscular injection. Intramuscular injection can be combined with electroporation, i.e., application of an electric field to facilitate delivery of the DNA plasmids to cells. As used herein, the term “electroporation” refers to the use of a transmembrane electric field pulse to induce microscopic pathways (pores) in a bio-membrane. During in vivo electroporation, electrical fields of appropriate magnitude and duration are applied to cells, inducing a transient state of enhanced cell membrane permeability, thus enabling the cellular uptake of molecules unable to cross cell membranes on their own. Creation of such pores by electroporation facilitates passage of biomolecules, such as plasmids, oligonucleotides, siRNAs, drugs, etc., from one side of a cellular membrane to the other. In vivo electroporation for the delivery of DNA has been shown to significantly increase plasmid uptake by host cells, while also leading to mild-to-moderate inflammation at the injection site. As a result, transfection efficiency is significantly improved (e.g., up to 1,000-fold and 100-fold respectively) with intradermal or intramuscular electroporation, in comparison to conventional injection.

In a typical embodiment, electroporation is combined with intramuscular injection. However, it is also possible to combine electroporation with other forms of parenteral administration, e.g., intradermal injection, subcutaneous injection, etc.

Administration of a pharmaceutical composition, combination or vaccine of the application via electroporation can be accomplished using electroporation devices that can be configured to deliver to a desired tissue of a mammal a pulse of energy effective to cause reversible pores to form in cell membranes. The electroporation device can include an electroporation component and an electrode assembly or handle assembly. The electroporation component can include one or more of the following components of electroporation devices: controller, current waveform generator, impedance tester, waveform logger, input element, status reporting element, communication port, memory component, power source, and power switch. Electroporation can be accomplished using an in vivo electroporation device. Examples of electroporation devices and electroporation methods that can facilitate delivery of compositions and immunogenic combinations of the application, particularly those comprising DNA plasmids, include CELLECTRA® (Inovio Pharmaceuticals, Blue Bell, PA), Elgen electroporator (Inovio Pharmaceuticals, Inc.) Tri-Grid™ delivery system (Ichor Medical Systems, Inc., San Diego, CA 92121) and those described in U.S. Pat. Nos. 7,664,545, 8,209,006, 9,452,285, 5,273,525, 6,110,161, 6,261,281, 6,958,060, and 6,939,862, 7,328,064, 6,041,252, 5,873,849, 6,278,895, 6,319,901, 6,912,417, 8,187,249, 9,364,664, 9,802,035, 6,117,660, and International Patent Application Publication WO2017172838, all of which are herein incorporated by reference in their entireties. Also contemplated by the application for delivery of the compositions and combinations of the application are use of a pulsed electric field, for instance as described in, e.g., U.S. Pat. No. 6,697,669, which is herein incorporated by reference in its entirety.

In other embodiments of the application in which a pharmaceutical composition or combination comprises one or more DNA plasmids, the method of administration is transdermal. Transdermal administration can be combined with epidermal skin abrasion to facilitate delivery of the DNA plasmids to cells. For example, a dermatological patch can be used for epidermal skin abrasion. Upon removal of the dermatological patch, the composition or combination can be deposited on the abraised skin.

Methods of delivery are not limited to the above described embodiments, and any means for intracellular delivery can be used. Other methods of intracellular delivery contemplated by the methods of the application include, but are not limited to, liposome encapsulation, lipoplexes, nanoparticles, etc. For example, an mRNA encoding one or more HBV TALENs of the application can be formulated in a composition that comprises one or more lipid molecules, preferably positively charged lipid molecules. In some embodiments, an mRNA encoding one or more HBV TALENs of the disclosure can be formulated using one or more liposomes, lipoplexes, and/or lipid nanoparticles. In some embodiments, liposome or lipid nanoparticle formulations described herein can comprise a polycationic composition. In some embodiments, the formulations comprising a polycationic composition can be used for the delivery of the HBV TALENs described herein in vivo and/or ex vitro.

### Embodiments

Embodiment 1 comprises a method for treating hepatitis infection in a subject in need thereof, comprising administering to the subject a combination of mRNA molecules comprising:
(1) a first mRNA molecule comprising a polynucleotide sequence encoding a first transcription activator like effector nuclease (TALEN) monomer comprising a first TALE DNA binding domain and a first nuclease catalytic domain, wherein the first TALE DNA binding domain is capable of binding to a first half-site sequence of a target nucleic acid sequence within an HBV genome; and
(2) a second mRNA molecule comprising a polynucleotide sequence encoding a second TALEN monomer comprising a second TALE DNA binding domain and a second nuclease catalytic domain, wherein the second TALE DNA binding domain is capable of binding to a second half-site sequence of the target nucleic acid sequence;
wherein the first TALEN monomer and the second TALEN monomer are capable of forming a dimer that cleaves the target nucleic acid sequence when the first TALE DNA binding domain binds to the first half-site sequence and the second TALE DNA binding domain binds to the second half-site sequence,
preferably, the first nuclease catalytic domain is a first FokI nuclease catalytic domain and the second nuclease catalytic domain is a second FokI nuclease catalytic domain.

Embodiment 1a comprises the method of embodiment 1, wherein the first FokI nuclease catalytic domain and the second FokI nuclease catalytic domain are the same.

Embodiment 1b comprises the method of embodiment 1, wherein the first FokI nuclease catalytic domain and the second FokI nuclease catalytic domain are different.

Embodiment 2 comprises the method of any one of embodiments 1-1b, wherein the target nucleic acid sequence is within the sequence that encodes HBsAg and HBV polymerase (pol).

Embodiment 2a comprises the method of embodiment 2, wherein the first half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 1, and the second half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 2.

Embodiment 2b comprises the method of embodiment 2, wherein the first half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 3, and the second half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 4.

Embodiment 2c comprises the method of embodiment 2, wherein the target nucleic acid sequence comprises the polynucleotide sequence at least 90% identical, such as at least 90%, 91%, 92%, 93%, 94%, 95%, 96%, 97%, 98%, 99% or 100% identical, to the nucleic acid sequence of SEQ ID NO: 5 or SEQ ID NO: 6.

Embodiment 3 comprises the method of any one of embodiments 1-2c, wherein the first and second mRNA molecules each further comprise one or more, preferably all, of a 5′ cap, a 5′-UTR, a sequence encoding a nuclear localization signal, a sequence encoding an N-terminal domain, a sequence encoding a C-terminal domain, a 3′-UTR, and a poly adenosine tail.

Embodiment 3a comprises the method of embodiment 3, wherein the first and second mRNA molecules each comprise a 5′ cap, a 5′-UTR, a sequence encoding an N-terminal domain, a sequence encoding a C-terminal domain, a 3′-UTR, and a poly adenosine tail.

Embodiment 3b comprises the method of embodiment 3, wherein the first and second mRNA molecules each comprise a 5′ cap, a 5′-UTR, a sequence encoding a nuclear localization signal, a sequence encoding an N-terminal domain, a sequence encoding a C-terminal domain, a 3′-UTR, and a poly adenosine tail.

Embodiment 4 comprises the method of any one of embodiments 1-3b, wherein the first TALE DNA binding domain comprises an amino acid sequence at least 90% identical to SEQ ID NO: 7 or SEQ ID NO: 9, the second TALE DNA binding domain comprises an amino acid sequence at least 90% identical to SEQ ID NO: 8 or SEQ ID NO: 10, respectively, and the first and second FokI nuclease catalytic domain each comprise an amino acid sequence at least 90% identical to SEQ ID NO: 11.

Embodiment 4a comprises the method of embodiment 4, wherein the first TALE DNA binding domain comprises an amino acid sequence at least 90% identical to SEQ ID NO: 7, the second TALE DNA binding domain comprises an amino acid sequence at least 90% identical to SEQ ID NO: 8, and the first and second FokI nuclease catalytic domain each comprise an amino acid sequence at least 90% identical to SEQ ID NO: 11.

Embodiment 4a1 comprises the method of embodiment 4a, wherein the first TALE DNA binding domain comprises the amino acid sequence of SEQ ID NO: 7, the second TALE DNA binding domain comprises the amino acid sequence of SEQ ID NO: 8, and the first and second FokI nuclease catalytic domain each comprise the amino acid sequence of SEQ ID NO: 11.

Embodiment 4a2 comprises the method of embodiment 4a, wherein the first TALE DNA binding domain consists of the amino acid sequence of SEQ ID NO: 7, the second TALE DNA binding domain consists of the amino acid sequence of SEQ ID NO: 8, and the first and second FokI nuclease catalytic domain each consist of the amino acid sequence of SEQ ID NO: 11.

Embodiment 4b comprises the method of embodiment 4, wherein the first TALE DNA binding domain comprises an amino acid sequence at least 90% identical to SEQ ID NO: 9, the second TALE DNA binding domain comprises an amino acid sequence at least 90% identical to SEQ ID NO: 10, and the first and second FokI nuclease catalytic domain each comprise an amino acid sequence at least 90% identical to SEQ ID NO: 11.

Embodiment 4b1 comprises the method of embodiment 4b, wherein the first TALE DNA binding domain comprises the amino acid sequence of SEQ ID NO: 9, the second TALE DNA binding domain comprises the amino acid sequence of SEQ ID NO: 10, and the first and second FokI nuclease catalytic domain each comprise the amino acid sequence of SEQ ID NO: 11.

Embodiment 4b2 comprises the method of embodiment 4b, wherein the first TALE DNA binding domain consists of the amino acid sequence of SEQ ID NO: 9, the second TALE DNA binding domain consists of the amino acid sequence of SEQ ID NO: 10, and the first and second FokI nuclease catalytic domain each consist of the amino acid sequence of SEQ ID NO: 11.

Embodiment 5 comprises the method of any one of embodiments 1-4b2, wherein the HBV genome is of one or more of genotypes A, B, C, D, E, F, G, H, I and J.

Embodiment 5a comprises the method of embodiment 5, wherein the HBV genome is of one or more of genotypes A, B, C, D, E, F, G and H.

Embodiment 6 comprises the method of any one of embodiments 1-5a, further comprising administering to the subject a second therapeutic composition, preferably comprising an anti-HBV agent.

Embodiment 7 comprises the method of any one of embodiments 1-6, wherein the subject has an HBV infection, preferably a chronic HBV infection.

Embodiment 8 comprises the method of any one of embodiments 1-7, wherein the subject is co-infected with HBV and HDV.

Embodiment 9 comprises a composition comprising a combination of mRNA molecules encapsulated in lipid nanoparticles comprising:
(1) a first mRNA molecule comprising a polynucleotide sequence encoding a first transcription activator like effector nuclease (TALEN) monomer comprising a first TALE DNA binding domain and a first nuclease catalytic domain, wherein the first TALE DNA binding domain is capable of binding to a first half-site sequence of a target nucleic acid sequence within an HBV genome; and
(2) a second mRNA molecule comprising a polynucleotide sequence encoding a second TALEN monomer comprising a second TALE DNA binding domain and a second nuclease catalytic domain, wherein the second TALE DNA binding domain is capable of binding to a second half-site sequence of the target nucleic acid sequence;
wherein the first TALEN monomer and the second TALEN monomer are capable of forming a dimer that cleaves the target nucleic acid sequence when the first TALE DNA binding domain binds to the first half-site sequence and the second TALE DNA binding domain binds to the second half-site sequence,
preferably, the first nuclease catalytic domain is a first FokI nuclease catalytic domain and the second nuclease catalytic domain is a second FokI nuclease catalytic domain.

Embodiment 9a comprises the composition of embodiment 9, wherein the first FokI nuclease catalytic domain and the second FokI nuclease catalytic domain are the same.

Embodiment 9b comprises the composition of embodiment 9, wherein the first FokI nuclease catalytic domain and the second FokI nuclease catalytic domain are different.

Embodiment 9c comprises the composition of any one of embodiments 9-9b, wherein each mRNA molecule is individually encapsulated in a lipid nanoparticle.

Embodiment 9d comprises the composition of any one of embodiments 9-9b, wherein multiple mRNA molecules are encapsulated in an individual lipid nanoparticle.

Embodiment 10 comprises the composition of any one of embodiments 9-9d, wherein the target nucleic acid sequence is within the sequence that encodes HBsAg and HBV polymerase (pol).

Embodiment 10a comprises the composition of embodiment 10, wherein the first half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 1, and the second half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 2.

Embodiment 10b comprises the composition of embodiment 10, wherein the first half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 3, and the second half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 4.

Embodiment 10c comprises the composition of embodiment 10, wherein the target nucleic acid sequence comprises the polynucleotide sequence at least 90% identical, such as at least 90%, 91%, 92%, 93%, 94%, 95%, 96%, 97%, 98%, 99% or 100% identical, to the nucleic acid sequence of SEQ ID NO: 5 or SEQ ID NO: 6.

Embodiment 11 comprises the composition of any one of embodiments 9-10c, wherein the first and second mRNA molecules each further comprise one or more of a 5′ cap, a 5′-UTR, a sequence encoding a nuclear localization signal, a sequence encoding an N-terminal domain, a sequence encoding a C-terminal domain, a 3′-UTR, and a poly adenosine tail.

Embodiment 12 comprises the composition of any one of embodiments 9-11, wherein the first TALE DNA binding domain comprises an amino acid sequence at least 90% identical to SEQ ID NO: 7 or SEQ ID NO: 9, the second TALE DNA binding domain comprises an amino acid sequence at least 90% identical to SEQ ID NO: 8 or SEQ ID NO: 10, respectively, and the first and second FokI nuclease catalytic domain each comprise an amino acid sequence at least 90% identical to SEQ ID NO: 11.

Embodiment 12a comprises the composition of embodiment 12, wherein the first TALE DNA binding domain comprises an amino acid sequence at least 90% identical to SEQ ID NO: 7, the second TALE DNA binding domain comprises an amino acid sequence at least 90% identical to SEQ ID NO: 8, and the first and second FokI nuclease catalytic domain each comprise an amino acid sequence at least 90% identical to SEQ ID NO: 11.

Embodiment 12a1 comprises the composition of embodiment 12a, wherein the first TALE DNA binding domain comprises the amino acid sequence of SEQ ID NO: 7, the second TALE DNA binding domain comprises the amino acid sequence of SEQ ID NO: 8, and the first and second FokI nuclease catalytic domain each comprise the amino acid sequence of SEQ ID NO: 11.

Embodiment 12a2 comprises the composition of embodiment 12a, wherein the first TALE DNA binding domain consists of the amino acid sequence of SEQ ID NO: 7, the second TALE DNA binding domain consists of the amino acid sequence of SEQ ID NO: 8, and the first and second FokI nuclease catalytic domain each consist of the amino acid sequence of SEQ ID NO: 11.

Embodiment 12b comprises the composition of embodiment 12, wherein the first TALE DNA binding domain comprises an amino acid sequence at least 90% identical to SEQ ID NO: 9, the second TALE DNA binding domain comprises an amino acid sequence at least 90% identical to SEQ ID NO: 10, and the first and second FokI nuclease catalytic domain each comprise an amino acid sequence at least 90% identical to SEQ ID NO: 11.

Embodiment 12b1 comprises the composition of embodiment 12b, wherein the first TALE DNA binding domain comprises the amino acid sequence of SEQ ID NO: 9, the second TALE DNA binding domain comprises the amino acid sequence of SEQ ID NO: 10, and the first and second FokI nuclease catalytic domain each comprise the amino acid sequence of SEQ ID NO: 11.

Embodiment 12b2 comprises the composition of embodiment 12b, wherein the first TALE DNA binding domain consists of the amino acid sequence of SEQ ID NO: 9, the second TALE DNA binding domain consists of the amino acid sequence of SEQ ID NO: 10, and the first and second FokI nuclease catalytic domain each consist of the amino acid sequence of SEQ ID NO: 11.

Embodiment 13 comprises the composition of any one of embodiments 9-12b2, wherein the HBV genome is of one or more of genotypes A, B, C, D, E, F, G, H, I and J.

Embodiment 13a comprises the composition of embodiment 13, wherein the HBV genome is of one or more of genotypes A, B, C, D, E, F, G and H.

Embodiment 14 comprises the composition of any one of embodiments 9-13a, wherein the lipid nanoparticles encapsulating the combination of mRNA molecules comprise a cationic lipid and at least one other lipid selected from the group consisting of anionic lipids, zwitterionic lipids, neutral lipids, steroids, polymer conjugated lipids, phospholipids, glycolipids, and combinations thereof.

Embodiment 15 comprises a combination of:
(1) a first nucleic acid, preferably a first mRNA molecule, comprising a polynucleotide sequence encoding a first transcription activator like effector nuclease (TALEN) monomer comprising a first TALE DNA binding domain and a first FokI nuclease catalytic domain, wherein the first TALE DNA binding domain is capable of binding to a first half-site sequence of a target nucleic acid sequence within an HBV genome, and the first half-site sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 1; and
(2) a second nucleic acid, preferably a second mRNA molecule, comprising a polynucleotide sequence encoding a second transcription activator like effector nuclease (TALEN) monomer comprising a second TALE DNA binding domain and a second FokI nuclease catalytic domain, wherein the second TALE DNA binding domain is capable of binding to a second half-site sequence of the target nucleic acid sequence, and the second half-site sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 2;
wherein the first TALEN monomer and the second TALEN monomer are capable of forming a dimer that cleaves the target nucleic acid sequence when the first TALE DNA binding domain binds to the first half-site sequence and the second TALE DNA binding domain binds to the second half-site sequence.

Embodiment 15a comprises the combination of embodiment 15, wherein the first half-site sequence comprises the nucleic acid sequence of SEQ ID NO: 1 and the second half-site sequence comprises the nucleic acid sequence of SEQ ID NO: 2.

Embodiment 15b comprises the combination of embodiment 15, wherein the first half-site sequence consists of the nucleic acid sequence of SEQ ID NO: 1 and the second half-site sequence consists of the nucleic acid sequence of SEQ ID NO: 2.

Embodiment 16 comprises the combination of any one of embodiments 15-15b, wherein the HBV genome is of one or more of genotypes A, B, C, D, E, F, G, H, I and J.

Embodiment 16a comprises the combination of embodiment 16, wherein the HBV genome is of one or more of genotypes A, B, C, D, E, F, G and H.

Embodiment 17 comprises a composition comprising the combination of any one of embodiments 15-16a, wherein the first and second nucleic acids are separately or jointly encapsulated in lipid nanoparticles.

Embodiment 18 comprises a nucleic acid molecule encoding at least one of the first mRNA molecule and the second mRNA molecule of any one of embodiments 15-15b.

Embodiment 19 comprises an isolated host cell comprising the nucleic acid molecule of embodiment 18.

Embodiment 20 comprises the isolated host cell of embodiment 19, wherein the host cell is a hepatocyte.

Embodiment 21 comprises a pharmaceutical composition comprising the composition of any one of embodiments 9-14 or 17, the combination of any one of embodiments 15-16a, the nucleic acid molecule of embodiment 18, or the isolated host cell of embodiment 19 or 20, and a pharmaceutically acceptable carrier.

Embodiment 22 comprises a method of cleaving a target nucleic acid sequence in an HBV genome, comprising contacting the HBV genome with the composition of any one of embodiments 9-14 or 17, the combination of any one of embodiments 15-16a, the nucleic acid molecule of embodiment 18, the isolated host cell of embodiment 19 or 20, or the pharmaceutical composition of embodiment 21.

Embodiment 23 comprises a method for inducing gene editing of a target nucleic acid sequence in an HBV genome, comprising contacting the HBV genome with the composition of any one of embodiments 9-14 or 17, the combination of any one of embodiments 15-16a, the nucleic acid molecule of embodiment 18, the isolated host cell of embodiment 19 or 20, or the pharmaceutical composition of embodiment 21.

Embodiment 24 comprises the method of embodiment 22 or 23, wherein the HBV genome is of one or more of genotypes A, B, C, D, E, F, G, H, I and J.

Embodiment 24a comprises the method of embodiment 24, wherein the HBV genome is of one or more of genotypes A, B, C, D, E, F, G and H.

Embodiment 25 comprises a method for treating a hepatitis infection in a subject in need thereof, comprising administering to the subject the pharmaceutical composition according to embodiment 21.

Embodiment 26 comprises a method for reducing infection and/or replication of HBV in a subject, comprising administering to the subject the pharmaceutical composition according to embodiment 21.

Embodiment 27 comprises the method of embodiment 25 or 26, further comprising administering to the subject a second therapeutic composition, preferably comprising an anti-HBV agent.

Embodiment 28 comprises the method of any one of embodiments 25-27, wherein the subject has an HBV infection, preferably a chronic HBV infection.

Embodiment 29 comprises the method of any one of embodiments 25-28, wherein the subject is co-infected with HBV and HDV.

Embodiment 30 comprises the method of any one of embodiments 25-29, wherein an expression level of one or more of HBsAg, HBeAg, HBV DNA, HBV cccDNA, or integrated HBV DNA is reduced in the subject.

Embodiment 30a comprises the method of embodiment 30, wherein serum and/or plasma level of one or more of HBsAg, HBeAg, and HBV DNA is reduced in the subject.

Embodiment 31 comprises the method of embodiment 30, wherein the expression level is a hepatocyte level, a nuclear or cellular level, a liver level, a serum level, or a plasma level.

Embodiment 32 comprises a method of producing a TALEN comprising transcribing the nucleic acid molecule of embodiment 18, in vitro or in vivo.

Embodiment 33 comprises the pharmaceutical composition of embodiment 21 for use in treating a hepatitis B virus (HBV)-induced disease in a subject in need thereof, preferably wherein the subject has chronic HBV infection.

Embodiment 33a comprises the pharmaceutical composition of embodiment 33, in combination with another therapeutic agent, preferably another anti-HBV agent.

Embodiment 34 comprises the pharmaceutical composition of embodiment 33 or 33a, wherein the HBV-induced disease is selected from the group consisting of advanced fibrosis, cirrhosis and hepatocellular carcinoma (HCC).

### Materials and Methods for Examples 1-6

### Cells and Culturing

Cryopreserved HepG2.2.15 cells (Fox Chase Cancer Center) were thawed and cultured in Dulbecco's Modified Eagle's Medium (DMEM) (Invitrogen) containing 10% FBS and supplemented with antibiotics, 2 mM L-Glutamine and G418. Cells were cultured on collagen I coated flasks and plates.

Cryopreserved primary human hepatocytes (PHHs) (Lonza, lot 8317) were thawed and cultured in Dulbecco's Modified Eagle's Medium (DMEM) (Invitrogen) containing 10% FBS and a supplement cocktail which includes HEPES, L-proline, insulin, epidermal growth factor, dexamethasone, ascorbic acid-2-phosphate, and 2% DMSO (Sigma).

### HBV Inoculum

Genotype (GT) D HBV inoculum was collected and concentrated from supernatants of HepG2.2.15 human hepatoblastoma cells, which constitutively express GT D HBV (Genbank U95551). GT A (PS 3.57), B (PS 3.4), C (PS 3.7), and D (PS 3.56) HBV inoculum from patient sera were purchased from American Red Cross.

### HBV DNA Plasmids

Plasmid DNA containing a 1.1× genotype B HBV genome under the control of a CMV promoter was previously cloned from serum of an HBV infected patient (Genbank AY220698, Fudan University, China). HBV sequences from genotype A (Genbank AF305422), C (Genbank AB033550), D (Genbank V01460), E (Genbank AB274971), F (Genbank AB036912), G (Genbank AF160501), and H (Genbank AB375159) were gene synthesized as 1.1× genome, and included part of the CMV promoter at the 5′-end. The gene-synthesized piece was confirmed by sequencing and subcloned into the pCMV-HBV plasmid using restriction sites SalI and NdeI (Genewiz, USA).

HBV TALEN mRNA

TALEN DNA sequences were cloned into plasmids with a T7 promoter for in vitro transcription. Plasmids were linearized using BspQI restriction site and in vitro transcribed. Briefly, nucleotide triphosphates (NTPs) and T7 RNA polymerase were combined with the linearized template in an aqueous buffered solution and allowed to incubate. The resultant mRNA transcript was purified using column chromatography, DNA and double stranded RNA contamination was removed from mRNA using an enzymatic reaction, mRNA was concentrated, and buffer was exchanged.

Transfection of HBV TALEN mRNA Using HepG2.2.15 Cells

On the day of transfection, mRNA encoding the right or the left TALEN protein were combined at a 1:1 ratio in OptiMEM media, prior to mixing with Lipofectamine MessengerMAX transfection reagents (Invitrogen). Transfection mixtures in the absence of HBV TALEN mRNAs were also prepared as no TALEN control. Transfection mixtures were placed on 96 well plate wells for reverse transfection of HepG2.2.15 cells grown that were seeded on top of the transfection mixtures at a density of 30,000/well and maintain at 37° C. overnight, after which media containing the transfection mixtures was removed and replenished with fresh cell cultured media. Transfected cells were maintained at 30° C., with another media change on day 3 post transfection. Gene editing and secreted HBsAg were monitored on day 6 post transfection.

HBV Infection and Transfection of HBV TALEN mRNA Using PHHs

PHHs were seeded at a density of 240,000 cells/well in a collagen coated 24-well plate at least one day prior to HBV infection. PHHs were infected with HBV inoculum at 200 genome equivalent per cell in medium containing 4% PEG-8000 (Sigma). HBV inoculum was removed the next day and cccDNA and viral replication were allowed to establish for at least 5 days prior to transfection of HBV TALEN encoding mRNA. On the day of transfection, mRNA encoding the right or the left TALEN protein was combined at a 1:1 ratio in OptiMEM media, prior to mixing with Lipofectamine messenger Max transfection reagents (Invitrogen). Transfection mixtures in the absence of HBV TALEN mRNAs were also prepared as no-TALEN control. HBV infected PHHs were transfected with or without HBV TALEN mRNAs at 37° C. overnight, after which media containing the transfection mixtures was removed and replenished with fresh PHH cultured media. Transfected cells were maintained at 30° C., with another media change on day 3 post transfection. Gene editing and secreted HBsAg and HBeAg were monitored on day 6 post transfection.

### HBV TALEN Protein and HBV Antigen Detection in Cell Culture

Expression of HBV TALEN protein in transfected PHHs were detected by immunofluorescence at 6-hour post transfection. Cells were fixed with Perfusion Fixative (Electron Microscopy Sciences, VWR), permeabilized by using 1% Triton-X 100 and blocked with 3% normal goat serum (NGS) in PBS. Immunostaining was performed by using a primary peptide tag-specific antibody containing 3% NGS followed by secondary staining with goat anti-mouse Alexa Fluor 488 (Invitrogen) diluted 1:1000 in PBS containing 3% NGS. Nuclei staining was done in conjunction with secondary staining where DAPI (ThermoFisher), diluted at 1:1000, was mixed with the secondary antibody. Levels of HBsAg and HBeAg were monitored from the supernatants of HBV infected PHHs or HepG2.2.15 cells by using Chemiluminescent Immunoassay Kits (Autobio Diagnostics) corresponding to the quantitative detection of HBsAg and HBeAg, respectively. Immunofluorescence staining and imaging was performed on the Opera system (Perkin Elmer).

HBV cccDNA Gene Editing Detection

On day 6 post transfection of HBV TALEN mRNAs, PHHs were treated for 10 minutes at room temperature with lysis buffer containing 50 mM Tris-HCl, pH 7.5, 150 mM NaCl, 1% NP40. Cells were spun down to isolate the nuclei from which genomic and HBV DNA were extracted by using the NucleoSpin Tissue Kit (Macherey-Nagel). To remove the relaxed circular form of HBV DNA from cccDNA, the extracted DNA was treated with 10 units of T5 exonuclease (New England Biolabs) at 37° C. for 60 minutes. T5 exonuclease was then inactivated by heating at 90° C. for 5 minutes. The targeted HBV sequence within the HBsAg encoding region was amplified by using forward primer 5′-CCT AGG ACC CCT TCT CGT GT (SEQ ID NO: 29) and reverse primer 5′-ACT GAG CCA GGA GAA ACG GG (SEQ ID NO: 30) and Platinum PCR SuperMix High Fidelity (Invitrogen) using the cycling condition of 1 cycle of 95° C. for 10 minutes, 45 cycles of 95° C. for 30 seconds (denaturing step), 64° C. for 30 seconds (annealing step), 72° C. for 30 seconds (extension step), followed by a final extension at 72° C. for 10 minutes. 10×NEB2 buffer and water was added to 10 uL of the PCR reaction to a final concentration of 1× and denatured at 95° C. for 10 minute, and then re-annealed by decreasing the temperature first to 85° C. at 3° C. per second and then decreasing further to 25° C. at 0.3° C. per second. Mismatched DNA sequences were cleaved by the addition of 5 units of T7E1 nuclease (New England Biolabs) and incubating at 37° C. for 1 hour. Digested and undigested products were analyzed by electrophoresis using Novex 10% TBE gel (Thermo Fisher) and staining with SYBR green (Thermo Fisher) for visualization by UV using the FluorChem M imager (Protein Simple). In addition, amplicons were submitted for next generation sequencing (Genewiz) to determine changes within the TALEN target region.

Formulation of HBV TALEN Encoding mRNA within Lipid Nanoparticles

Lipid encapsulated mRNA particles were prepared by mixing lipids (cationic lipid: DSPC: Cholesterol: PEG-DMG) in ethanol with mRNA encoding the desired polypeptide dissolved in Citrate buffer. The mixed material was instantaneously diluted with Phosphate Buffer. Ethanol was removed by dialysis against phosphate buffer using regenerated cellulose membrane (100 kD MWCO) or by tangential flow filtration (TFF) using modified polyethersulfone (mPES) hollow fiber membranes (100 kD MWCO). Once the ethanol was completely removed, the buffer was exchanged with HEPES (4-(2-hydroxyethyl)-1-piperazineethanesulfonic acid) buffer containing 50 mM NaCl and 9% sucrose, pH 7.3. The formulation was concentrated followed by 0.2 μm filtration using PES filters. The mRNA concentration in the formulation was then measured by Ribogreen fluorimetric assay following which the concentration was adjusted to a final desired concentration by diluting with HEPES buffer containing 50 mM NaCl, 9% sucrose, pH 7.3 containing glycerol. The final formulation was then filtered through a 0.2 μm filter and filled into glass vials, stoppered, capped and placed at −70±5° C. The frozen formulations were characterized for their mRNA content and percent encapsulation by Ribogreen assay, mRNA integrity by fragment analyzer, lipid content by high performance liquid chromatography (HPLC), particle size by dynamic light scattering on a Malvern Zetasizer Nano ZS, pH and Osmolality.

### AAV-HBV Infected Mouse Model

C57Bl6 male mice (Taconic) were infected with 1×10\hat{ }11 adenoviral infectious particles of AAV-1.3×HBV (FivePlus) by intravenous tail injection. Four weeks after infection, blood serum was collected and analyzed to quantify HBsAg and HBeAg levels. Mice with 4 Logs or more of HBsAg were selected for efficacy in vivo studies.

### HBV Antigens and HBV DNA Detection and Quantification in Blood Serum

Levels of HBsAg and HBeAg in the mouse blood serum of AAV-HBV mice were measured by using Chemiluminescent Immunoassay Kits (Autobio Diagnostics). Blood serum was diluted 1:100 and 1:250 in PBS for HBeAg and HBsAg CLIA detection, respectively.

HBV DNA was extracted from 5 μl of blood serum following instructions from QIAamp DNA Mini kit (Qiagen, Cat. #51306), appendix protocol for viral DNA. Briefly, after incubating for 10 min at 56 C in the proteinase K buffer, samples were mixed with 100% ethanol and applied to a QIAmp Mini Spin column. Spin columns were centrifuged and wash as indicated in the instructions and the isolated DNA was resuspended in 30 μl of water.

Purified serum HBV DNA was amplified using 2× TaqMan Advance Fast Mix (Thermo Fisher, Cat. #4444557) mixed with 20×sAg probe (Thermo Fisher, Assay Id Vi03453406_s1 TaqMan Gene Expression Assay (FAM) Dye Label and Assay Concentration FAM-MGB) and run using the QuantStudio 6 Flex qPCR machine and using the cycling condition of 1 cycle of 50° C. for 2 minutes and 95° C. for 2 minutes, 45 cycles of 95° C. for 10 seconds (denaturing step), 60° C. for 20 seconds (annealing step), 72° C. for 30 seconds (extension step) with a ramp speed of 1.6° C./second. For quantification a standard curve was run in parallel using serial dilutions of known copy numbers of synthetic HBV DNA by AATC (Quantitative Synthetic Hepatitis B virus DNA (ATCC® VR-3232SD™). Data was extracted and analyze using QuantStudio software, while statistical analysis was performed using GraphPad software.

### HBV DNA Gene Editing Detection in Mouse Liver Tissue

For gene editing analysis, HBV DNA was extracted from snap frozen mouse livers using reagents and protocol from Quick-DNA miniprep Plus Kit (ZymoResearch Cat. #D4068). First, snap frozen mouse liver tissue was placed in proteinase K digestion buffer and homogenized by both mechanical disruption, using tubes from Precellys Lysing kit 0.5 ml for soft tissue (Precellys, Cat. #P000933-LYSK0-A) in a Precellys Evolution Homogenizer (Precellys, Bertin Instruments). and then by digestion, incubating samples 20 minutes at 55° C. Then HBV DNA was purified as indicated in the Quick-DNA miniprep Plus Kit by spin columns clean up. Genomic DNA was eluted in 50 ul of DNA elution buffer and quantified.

HBV DNA target sequence was amplified by PCR using 5 ng/ul of purified DNA mixed in 25 ul PCR reactions with 2× SuperFI Platinum MasterMix (Invitrogen, Cat. #12358250); forward primer 5′-ACA CTC TIT CCC TAC ACG ACG CTC TTC CGA TCT TAT CGC TGG ATG TGT CTG CG-3′ (SEQ ID NO: 29) and reverse primer 5′-GAC TGG AGT TCA GAC GTG TGC TCT TCC GAT CTG TCC GAA GGT TTG GTA CAG C-3′ (SEQ ID NO: 30) and using the cycling condition of 1 cycle of 98° C. for 2 minutes, 35 cycles of 95° C. for 15 seconds (denaturing step), 60° C. for 15 seconds (annealing step), 72° C. for 30 seconds (extension step), followed by a final extension at 72° C. for 5 minutes. PCR products were cleaned up with NucleoSpin® Gel and PCR Clean-up kit (Macherey-Nagel) following kit instructions, eluted in NE buffer, quantified and diluted to 20 ng/ul as required for NGS sequencing. Samples were submitted for Amplicon sequencing (Amplicon-EZ) and analysis at Genewiz.

### EXAMPLES

### Example 1: TALEN Target Site and TALEN Structure

Publicly available bioinformatic tools were used to predict potential TALEN target sites within a consensus HBV genome sequence. TALEN target sites were selected from within the gene that encodes HBsAg/pol to reduce HBsAg secretion and viral replication (FIG. 1A). TALEN 28 and TALEN 33 target HBV genome regions of 50 and 53 base pairs in length, respectively, including the left arm TALEN sequence, right arm TALEN sequence and spacer sequence between both arms (FIG. 1B). All TALEN proteins were encoded by mRNA molecules that included a cap, a 5′ untranslated region (UTR), a sequence encoding a tag peptide, a sequence encoding a nuclear localization signal, a sequence encoding a TALEN N-terminal domain, a sequence encoding a TALEN DNA binding domain with the repeat domains that contain the repeat variable di-residues (RVD), a sequence encoding a C-terminal domain, a sequence encoding a FokI nuclease, a 3′ UTR and a polyA tail. Optionally, constructs including a cap, a 5′ untranslated region (UTR), a sequence encoding a nuclear localization signal, a sequence encoding a TALEN N-terminal domain, a sequence encoding a TALEN DNA binding domain with the repeat domains that contain the repeat variable di-residues (RVD), a sequence encoding a C-terminal domain, a sequence encoding a FokI nuclease, a 3′ UTR and a polyA tail can be made. TALEN 28 and TALEN 33 RVDs used to target the four DNA nucleotide bases were NN (targets A and G), NI (targets T), NG (targets C) and HD (targets G) (FIG. 1C).

### Example 2: TALEN Target Sites are Conserved and TALEN Show Broad Functional Genotype Coverage

Conservation analysis of the HBV target sequences for the TALEN 28 and 33 left and right arms was performed across hundreds of HBV genome sequences including all the different HBV genotypes. Alignment of the HBV target sequences across the different genotypes showed very high conservation, from 97-100% for TALEN 28 and TALEN 33 (Table 1 and Table 2 below, respectively). For sequences targeted by the right arm of TALEN 28 and TALEN 33, two positions showed a mismatch for Genotypes A and C (Table 1 and Table 2 below).

TABLE 1
Percent similarity analysis of the left and right arms of TALEN 28 across HBV genotypes A to H
TALEN 28 Left Target Sequence (% similarity against HBV genotypes)

| HBV | number of | | | | | | | | | | |
|---|---|---|---|---|---|---|---|---|---|---|---|
| GT | isolates | T | G | C | T | G | C | T | A | T | G |
| All | 6341 | | | | | | | | | | |
| A | 855 | 100.0 | 99.9 | 100.0 | 99.8 | 100.0 | 99.9 | 99.9 | 99.9 | 99.9 | 99.9 |
| B | 1749 | 99.9 | 99.8 | 100.0 | 99.8 | 99.9 | 99.9 | 99.9 | 99.8 | 99.9 | 99.8 |
| C | 2179 | 99.9 | 99.9 | 99.9 | 99.8 | 100.0 | 100.0 | 99.8 | 100.0 | 100.0 | 100.0 |
| D | 954 | 100.0 | 100.0 | 100.0 | 100.0 | 97.5 | 99.9 | 99.8 | 99.9 | 100.0 | 99.9 |
| E | 290 | 100.0 | 100.0 | 100.0 | 100.0 | 100.0 | 99.3 | 100.0 | 100.0 | 100.0 | 100.0 |
| F | 248 | 100.0 | 100.0 | 100.0 | 100.0 | 100.0 | 100.0 | 99.6 | 100.0 | 100.0 | 100.0 |
| G | 40 | 100.0 | 100.0 | 100.0 | 100.0 | 100.0 | 100.0 | 100.0 | 100.0 | 100.0 | 100.0 |
| H | 26 | 100.0 | 100.0 | 100.0 | 100.0 | 100.0 | 100.0 | 100.0 | 100.0 | 96.2 | 100.0 |

TALEN 28 Left Target Sequence (% similarity against HBV genotypes)

| HBV | number of | | | | | | | | | | |
|---|---|---|---|---|---|---|---|---|---|---|---|
| GT | isolates | C | C | T | C | A | T | C | T | T | C |
| All | 6341 | | | | | | | | | | |
| A | 855 | 99.8 | 99.9 | 99.8 | 100.0 | 99.9 | 100.0 | 100.0 | 99.9 | 100.0 | 100.0 |
| B | 1749 | 99.8 | 99.9 | 99.8 | 99.9 | 99.9 | 99.7 | 99.9 | 99.9 | 99.2 | 100.0 |
| C | 2179 | 100.0 | 100.0 | 99.7 | 99.9 | 99.9 | 99.0 | 100.0 | 100.0 | 99.8 | 100.0 |
| D | 954 | 99.9 | 100.0 | 100.0 | 99.9 | 100.0 | 98.8 | 100.0 | 100.0 | 99.9 | 99.9 |
| E | 290 | 100.0 | 100.0 | 100.0 | 100.0 | 100.0 | 100.0 | 100.0 | 100.0 | 100.0 | 100.0 |
| F | 248 | 100.0 | 100.0 | 100.0 | 100.0 | 100.0 | 99.6 | 100.0 | 100.0 | 100.0 | 100.0 |
| G | 40 | 100.0 | 100.0 | 100.0 | 100.0 | 100.0 | 100.0 | 100.0 | 100.0 | 100.0 | 100.0 |
| H | 26 | 100.0 | 100.0 | 100.0 | 100.0 | 100.0 | 100.0 | 100.0 | 100.0 | 100.0 | 100.0 |

TALEN 28 Right Target Sequence (% similarity against HBV genotypes)

| HBV | number of | | | | | | | | | | | | | | | | |
|---|---|---|---|---|---|---|---|---|---|---|---|---|---|---|---|---|---|
| GT | isolates | C | T | G | G | A | C | T | A | T | C | A | A | G | G | T | A |
| All | 6341 | | | | | | | | | | | | | | | | |
| A | 855 | 99.9 | 99.9 | 99.9 | 99.9 | 99.8 | 1.3 | 99.9 | 93.1 | 91.2 | 99.9 | 100.0 | 97.5 | 99.1 | 99.8 | 99.9 | 99.8 |
| B | 1749 | 99.8 | 99.9 | 99.9 | 99.9 | 99.9 | 99.5 | 99.9 | 98.7 | 99.3 | 99.9 | 99.8 | 97.6 | 99.9 | 99.9 | 99.9 | 99.9 |
| C | 2179 | 93.5 | 99.7 | 100.0 | 100.0 | 99.8 | 99.2 | 100.0 | 98.3 | 9.9 | 98.7 | 98.4 | 98.2 | 99.8 | 100.0 | 100.0 | 99.9 |
| D | 954 | 100.0 | 100.0 | 100.0 | 100.0 | 100.0 | 99.8 | 99.9 | 99.4 | 99.0 | 100.0 | 98.5 | 97.1 | 100.0 | 100.0 | 99.9 | 99.9 |
| E | 290 | 100.0 | 99.7 | 100.0 | 100.0 | 99.0 | 100.0 | 100.0 | 99.7 | 100.0 | 100.0 | 99.3 | 96.9 | 100.0 | 100.0 | 99.7 | 100.0 |
| F | 248 | 94.8 | 100.0 | 100.0 | 100.0 | 100.0 | 100.0 | 100.0 | 99.6 | 92.3 | 100.0 | 99.6 | 97.6 | 100.0 | 100.0 | 100.0 | 100.0 |
| G | 40 | 100.0 | 100.0 | 100.0 | 100.0 | 100.0 | 95.0 | 100.0 | 100.0 | 100.0 | 100.0 | 100.0 | 97.5 | 100.0 | 100.0 | 100.0 | 100.0 |
| H | 26 | 100.0 | 100.0 | 100.0 | 100.0 | 100.0 | 100.0 | 100.0 | 100.0 | 100.0 | 100.0 | 100.0 | 100.0 | 100.0 | 100.0 | 100.0 | 100.0 |

TABLE 2
Percent similarity analysis of the left and right
arms of TALEN 33 across HBV genotypes A to H
TALEN 33 Left Target Sequence (% similarity against HBV genotypes)

| HBV | number of | | | | | | | | | | |
|---|---|---|---|---|---|---|---|---|---|---|---|
| GT | isolates | T | C | C | T | G | C | T | G | C | T |
| All | 6341 | 99.9 | 99.9 | 100.0 | 99.9 | 99.9 | 100.0 | 99.8 | 99.6 | 99.9 | 99.9 |
| A | 855 | 99.9 | 100.0 | 100.0 | 100.0 | 99.9 | 100.0 | 99.8 | 100.0 | 99.9 | 99.9 |
| B | 1749 | 99.9 | 100.0 | 99.9 | 99.9 | 99.8 | 100.0 | 99.8 | 99.9 | 99.9 | 99.9 |
| C | 2179 | 99.8 | 99.8 | 100.0 | 99.9 | 100.0 | 99.9 | 99.8 | 100.0 | 100.0 | 99.8 |
| D | 954 | 99.8 | 100.0 | 99.9 | 100.0 | 100.0 | 100.0 | 100.0 | 99.9 | 99.9 | 99.8 |
| E | 290 | 100.0 | 100.0 | 100.0 | 100.0 | 100.0 | 100.0 | 100.0 | 99.3 | 99.3 | 100.0 |
| F | 248 | 100.0 | 100.0 | 100.0 | 100.0 | 100.0 | 100.0 | 100.0 | 100.0 | 100.0 | 99.6 |
| G | 40 | 100.0 | 100.0 | 100.0 | 100.0 | 100.0 | 100.0 | 100.0 | 100.0 | 100.0 | 100.0 |
| H | 26 | 100.0 | 100.0 | 100.0 | 100.0 | 100.0 | 100.0 | 100.0 | 100.0 | 100.0 | 100.0 |

TALEN 33 Left Target Sequence (% similarity against HBV genotypes)

| HBV | number of | | | | | | | | | |
|---|---|---|---|---|---|---|---|---|---|---|
| GT | isolates | A | T | G | C | C | T | C | A | T |
| All | 6341 | 99.9 | 99.9 | 99.9 | 99.9 | 100.0 | 99.8 | 99.9 | 99.9 | 99.4 |
| A | 855 | 99.9 | 99.9 | 99.9 | 99.8 | 99.9 | 99.8 | 100.0 | 99.9 | 100.0 |
| B | 1749 | 99.8 | 99.9 | 99.8 | 99.8 | 99.9 | 99.8 | 99.9 | 99.9 | 99.7 |
| C | 2179 | 100.0 | 100.0 | 100.0 | 100.0 | 100.0 | 99.7 | 99.9 | 99.9 | 99.0 |
| D | 954 | 99.9 | 100.0 | 99.9 | 99.9 | 100.0 | 100.0 | 99.9 | 100.0 | 98.8 |
| E | 290 | 100.0 | 100.0 | 100.0 | 100.0 | 100.0 | 100.0 | 100.0 | 100.0 | 100.0 |
| F | 248 | 100.0 | 100.0 | 100.0 | 100.0 | 100.0 | 100.0 | 100.0 | 100.0 | 99.6 |
| G | 40 | 100.0 | 100.0 | 100.0 | 100.0 | 100.0 | 100.0 | 100.0 | 100.0 | 100.0 |
| H | 26 | 100.0 | 96.2 | 100.0 | 100.0 | 100.0 | 100.0 | 100.0 | 100.0 | 100.0 |

TALEN 33 Right Target Sequence (% similarity against HBV genotypes)

| HBV | number of | | | | | | | | | | |
|---|---|---|---|---|---|---|---|---|---|---|---|
| GT | isolates | C | T | T | C | T | G | G | A | C | T |
| All | 6341 | 99.9 | 100.0 | 99.6 | 97.5 | 99.8 | 100.0 | 100.0 | 99.8 | 86.2 | 100.0 |
| A | 855 | 99.8 | 100.0 | 98.2 | 99.9 | 99.9 | 99.9 | 99.9 | 99.8 | 1.3 | 99.9 |
| B | 1749 | 99.9 | 100.0 | 99.9 | 99.8 | 99.9 | 99.9 | 99.9 | 99.9 | 99.5 | 99.9 |
| C | 2179 | 100.0 | 100.0 | 99.8 | 93.5 | 99.7 | 100.0 | 100.0 | 99.8 | 99.2 | 100.0 |
| D | 954 | 99.9 | 99.8 | 99.5 | 100.0 | 100.0 | 100.0 | 100.0 | 100.0 | 99.8 | 99.9 |
| E | 290 | 100.0 | 100.0 | 100.0 | 100.0 | 99.7 | 100.0 | 100.0 | 99.0 | 100.0 | 100.0 |
| F | 248 | 100.0 | 100.0 | 100.0 | 94.8 | 100.0 | 100.0 | 100.0 | 100.0 | 100.0 | 100.0 |
| G | 40 | 100.0 | 100.0 | 100.0 | 100.0 | 100.0 | 100.0 | 100.0 | 100.0 | 95.0 | 100.0 |
| H | 26 | 100.0 | 100.0 | 100.0 | 100.0 | 100.0 | 100.0 | 100.0 | 100.0 | 100.0 | 100.0 |

TALEN 33 Right Target Sequence (% similarity against HBV genotypes)

| HBV | number of | | | | | | | | | |
|---|---|---|---|---|---|---|---|---|---|---|
| GT | isolates | A | T | C | A | A | G | G | T | A |
| All | 6341 | 98.0 | 67.2 | 99.5 | 99.1 | 97.7 | 99.8 | 99.9 | 99.9 | 99.9 |
| A | 855 | 93.1 | 91.2 | 99.9 | 100.0 | 97.5 | 99.1 | 99.8 | 99.9 | 99.8 |
| B | 1749 | 98.7 | 99.3 | 99.9 | 99.8 | 97.6 | 99.9 | 99.9 | 99.9 | 99.9 |
| C | 2179 | 98.3 | 9.9 | 98.7 | 98.4 | 98.2 | 99.8 | 100.0 | 100.0 | 99.9 |
| D | 954 | 99.4 | 99.0 | 100.0 | 98.5 | 97.1 | 100.0 | 100.0 | 99.9 | 99.9 |
| E | 290 | 99.7 | 100.0 | 100.0 | 99.3 | 96.9 | 100.0 | 100.0 | 99.7 | 100.0 |
| F | 248 | 99.6 | 92.3 | 100.0 | 99.6 | 97.6 | 100.0 | 100.0 | 100.0 | 100.0 |
| G | 40 | 100.0 | 100.0 | 100.0 | 100.0 | 97.5 | 100.0 | 100.0 | 100.0 | 100.0 |
| H | 26 | 100.0 | 100.0 | 100.0 | 100.0 | 100.0 | 100.0 | 100.0 | 100.0 | 100.0 |

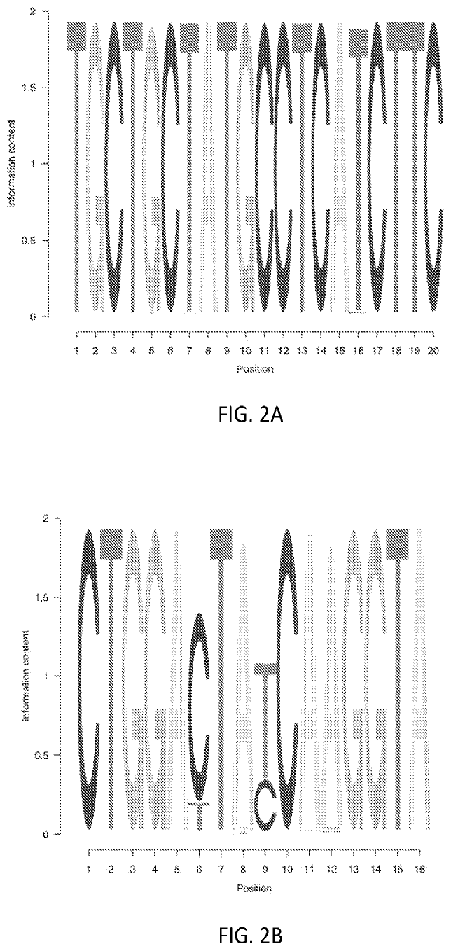

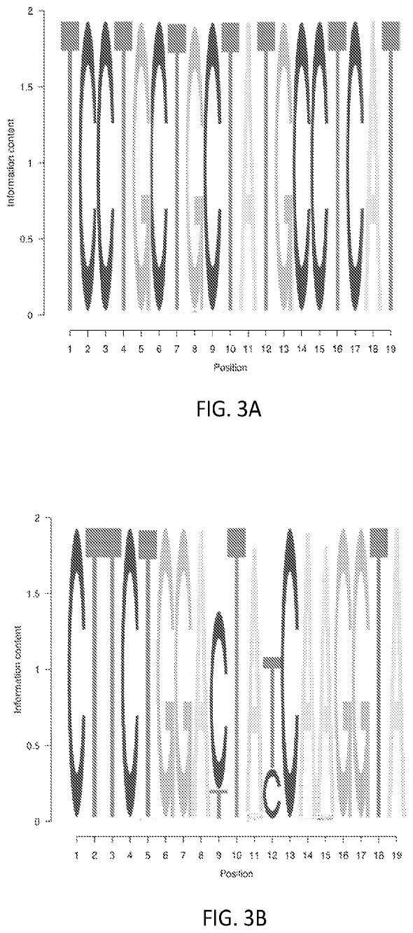

To graphically represent the conservation of sequences targeted by TALEN 28 and TALEN 33, conservation logos were generated based on the abundance of each targeted nucleotide in the different HBV genomes, where the higher the conservation, the larger the size of the abundant nucleotide(s). TALEN 28 and TALEN 33 left arms showed very high conservation across all the targeted nucleotides (FIG. 2A and FIG. 3A). For HBV sequences targeted by the right arm of both TALEN 28 and TALEN 33, two positions were found (positions 6 and 9 for TALEN 28, FIG. 2B; positions 9 and 12 for TALEN 33, FIG. 3B) that showed two alternative bases (C or T), depending on the HBV genotype.

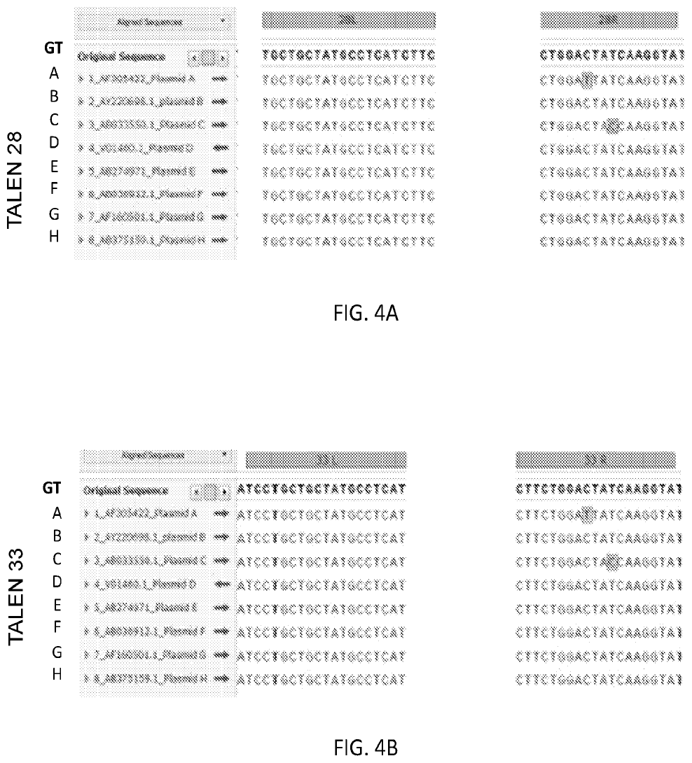

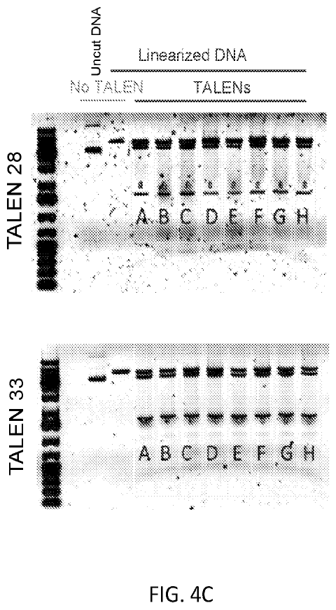

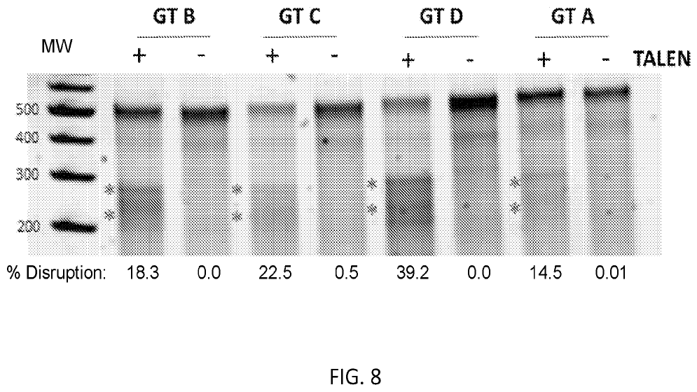

To understand how the variability of the HBV target sequence could affect TALEN efficacy, TALEN 28 and TALEN 33 genotype coverage was evaluated using a biochemical assay. HBV sequences from 8 different genotypes were cloned into plasmids and their TALEN 28 and TALEN 33 target sequences aligned, showing a single mismatch in Genotype A and C (FIG. 4A and FIG. 4B). Left and right arms from TALEN 28 and TALEN 33 mRNAs were translated in vitro using a reticulocyte in vitro translation system (Promega) and incubated with linearized plasmids containing sequences of the different HBV genotypes. TALEN digestion of plasmids containing HBV target sequences was analyzed using polyacrylamide gels and detected by the presence of a new DNA fragment that is absent in uncut DNA. TALEN 28 and TALEN 33 showed DNA cleavage for all HBV genotypes tested (A to H), thus displaying wide genotype coverage (FIG. 4C). Genotype coverage of TALEN 33 observed in the biochemical assay was validated by infecting primary human hepatocytes with HBV of different genotypes (A, B, C and D) and transfecting the cells with TALEN 33 mRNA. Disruption of the HBV DNA was observed in all samples transfected with TALEN 33 mRNA (FIG. 8, Table 3).

TABLE 3
TALEN 33 induced cccDNA disruptions from PHHs infected with HBV GT A to D*

| GT A (PS 3.57) HBsAg target sequence | Reads |
|---|---|
| TCCTGCTGCTATGCCTCATCTTCTTATTGGTTCTTCTGGATTATCAAGGTA | 40692 |
| TCCTGCTGCTATGCCTCATCTTCTTGTTGGTTCTTCTGGACTATCAAGGTA | 2017 |
| TCCTGCTGCTATGCCTCATCTTCT--TTGGTTCTTCTGGATTATCAAGGTA | 1934 |
| TCCTGCTGCTATGCCTCATCTTC-TATTGGTTCTTCTGGATTATCAAGGTA | 1069 |
| TCCTGCTGCTATGCCTCATCTTCTTA-TGGTTCTTCTGGATTATCAAGGTA | 1000 |
| TCCTGCTGCTATGCCTCATCTTC---TTGGTTCTTCTGGATTATCAAGGTA | 856 |
| GT B (PS 3.4) HBsAg target sequence | Reads |
| TCCTGCTGCTATGCCTCATCTTCTTGTTGGTTCTTCTGGACTATCAAGGTA | 66704 |
| TCCTGCTGCTATGCCTCATCTTCT--TTGGTTCTTCTGGACTATCAAGGTA | 4073 |
| TCCTGCTGCTATGCCTCATCTTC---TTGGTTCTTCTGGACTATCAAGGTA | 2355 |
| TCCTGCTGCTATGCCTCATCTTCTTG-TGGTTCTTCTGGACTATCAAGGTA | 2162 |
| TCCTGCTGCTATGCCTCATCTTC----TGGTTCTTCTGGACTATCAAGGTA | 1679 |
| TCCTGCTGCTATGCCTCATCTTC-TGTTGGTTCTTCTGGACTATCAAGGTA | 1074 |
| GT C (PS 3.7) HBsAg target sequence | Reads |
| TCCTGCTGCTATGCCTCATCTTCTTGTTGGTTCTTCTGGACTACCAAGGTA | 60493 |
| TCCTGCTGCTATGCCTCATCTTC-TGTTGGTTCTTCTGGACTACCAAGGTA | 4323 |
| TCCTGCTGCTATGCCTCATCTTCT--TTGGTTCTTCTGGACTACCAAGGTA | 3384 |
| TCCTGCTGCTATGCCTCATCTTC---TTGGTTCTTCTGGACTACCAAGGTA | 2863 |
| TCCTGCTGCTATGCCTCATCTTC----TGGTTCTTCTGGACTACCAAGGTA | 1087 |
| GT D (PS 3.56) HBsAg target sequence | Reads |
| TCCTGCTGCTATGCCTCATCTTCTTGTTGGTTCTTCTGGACTATCAAGGTA | 24933 |
| TCCTGCTGCTATGCCTCATCTTCT--TTGGTTCTTCTGGACTATCAAGGTA | 3649 |
| TCCTGCTGCTATGCCTCATCTTC---TTGGTTCTTCTGGACTATCAAGGTA | 3191 |
| TCCTGCTGCTATGCCTCATCTTC----TGGTTCTTCTGGACTATCAAGGTA | 1790 |
| TCCTGCTGCTATGCCTCATCTTC-TGTTGGTTCTTCTGGACTATCAAGGTA | 1672 |
| TCCTGCTGCTATGCCTCATCTTCTTG-TGGTTCTTCTGGACTATCAAGGTA | 1286 |
| TCCTGCTGCTATGCCTCATCTTCTT----GTTCTTCTGGACTATCAAGGTA | 497 |

| HBV | |
|---|---|
| gene^{#} | TCCTGCTGCTATGCCTCATCTTCTTGTTGGTTCTTCTGGACTATCAAGGTA |
| Pol | P A A M P H L L V G S S G L S R |
| HBsAg^{#} | L L L C L I F L L V L L D Y Q G |

*TALEN 33-induced targeted cccDNA sequence disruptions were examined by next generation sequencing of the targeted amplicons. The left and right TALEN targeting sequences are bolded and the spacer sequence underlined. Commonly detected disruptions and the corresponding number of sequencing reads are indicated. Dash lines represent deletions and base substitutions are shown as larger fonts.
^{#}Open reading frames for HBV Pol and HBsAg within TALEN 33 target sequence.

### Example 3: HBV TALEN HBsAg Reduction and Gene Editing in HepG2.2.15 Cells

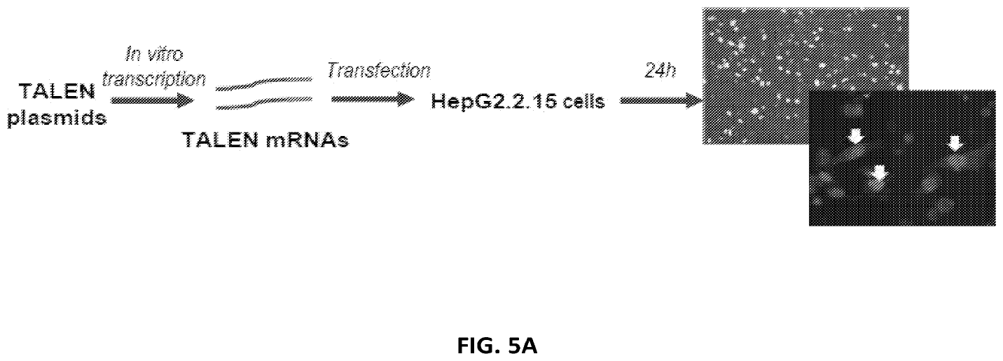

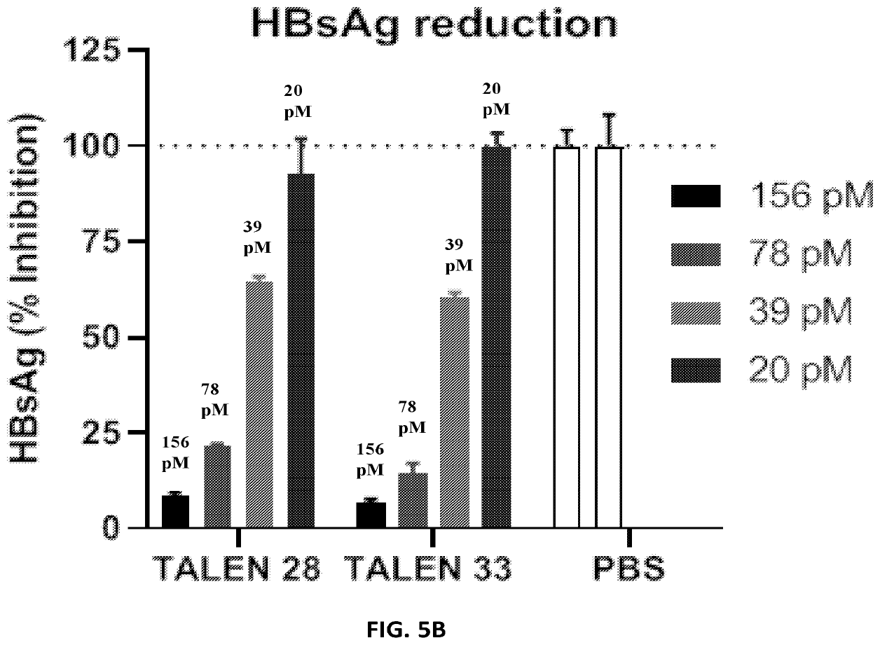

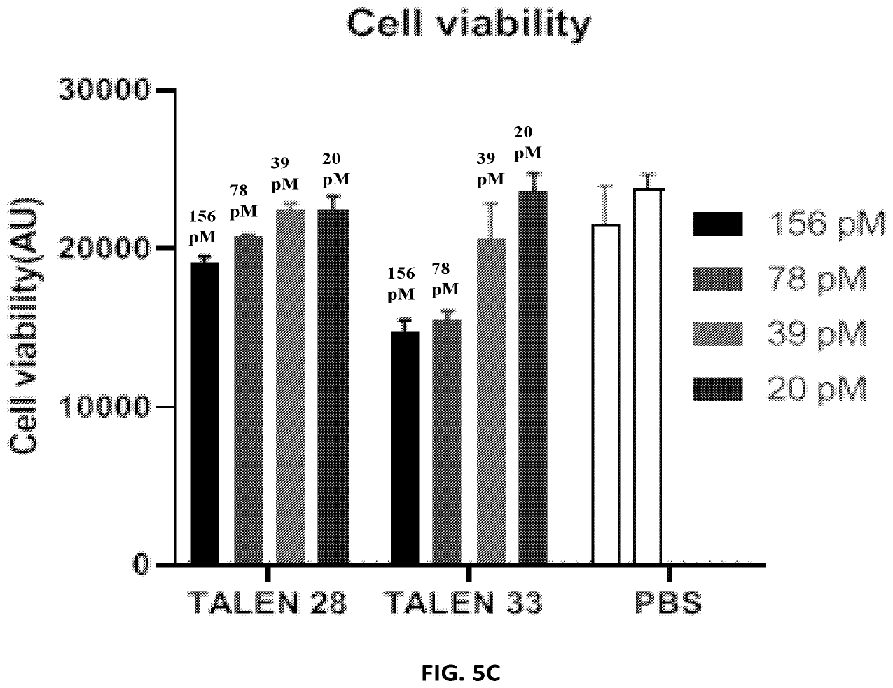

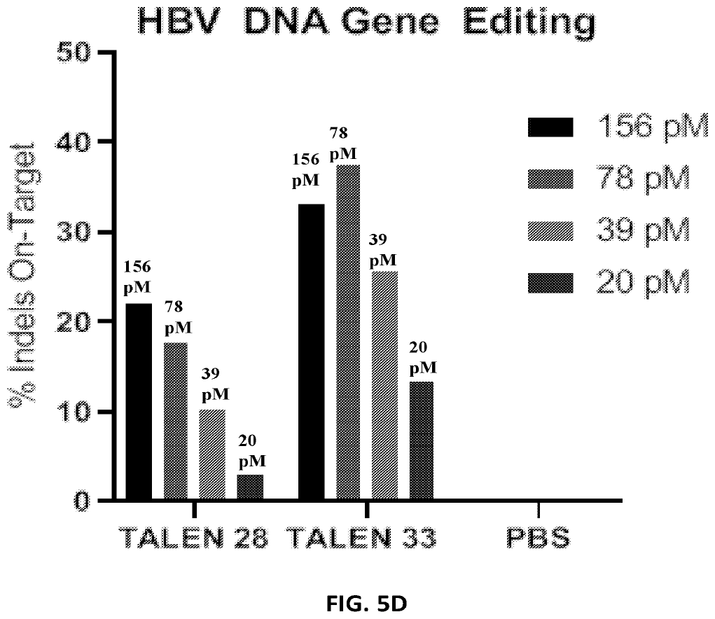

The efficacy of TALEN mRNA was tested in the HepG2.2.15 cell line that constitutively expresses HBV. Briefly, TALEN mRNA was transcribed in vitro from linearized TALEN plasmids. TALEN mRNA was transfected into HepG2.2.15 cells using Lipofectamine reagent. TALEN expression could be detected in the transfected cells after 24 hours by immunofluorescence using antibodies specific for TALEN proteins (FIG. 5A). TALEN 28 and TALEN 33 mRNAs were transfected into HepG2.2.15 cells at different concentrations and levels of HBsAg were evaluated in the cell culture media 6 days after transfection. Reduction in the levels of HBsAg in the cell culture media was dose-dependent (FIG. 5B). Transfection of high concentrations of mRNA had a negative effect on cell viability (FIG. 5C). HBV DNA editing activity was evaluated in the same samples by next generation sequencing 6 days after mRNA transfection. TALEN 28 and TALEN 33 gene editing activity was quantified by determining the abundance of insertions and deletions in the spacer region (FIG. 5D). As shown in FIG. 5D, gene editing activity was dose-dependent and correlated with HBsAg reduction.

### Example 4: HBV TALEN HBsAg Reduction and Gene Editing in Primary Human Hepatocytes (PHH)

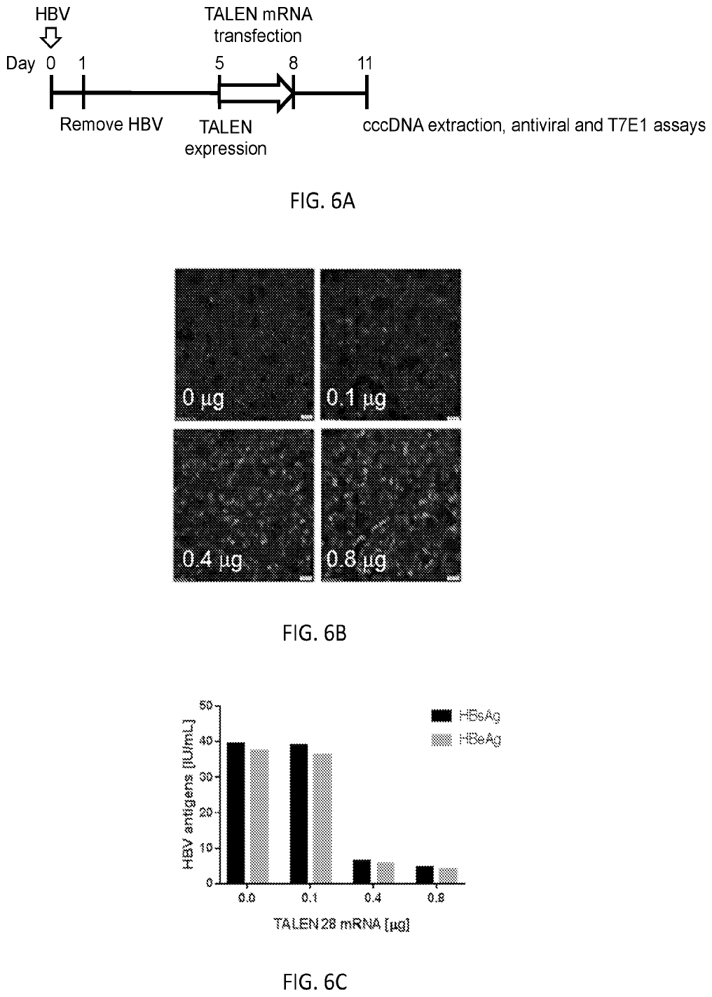

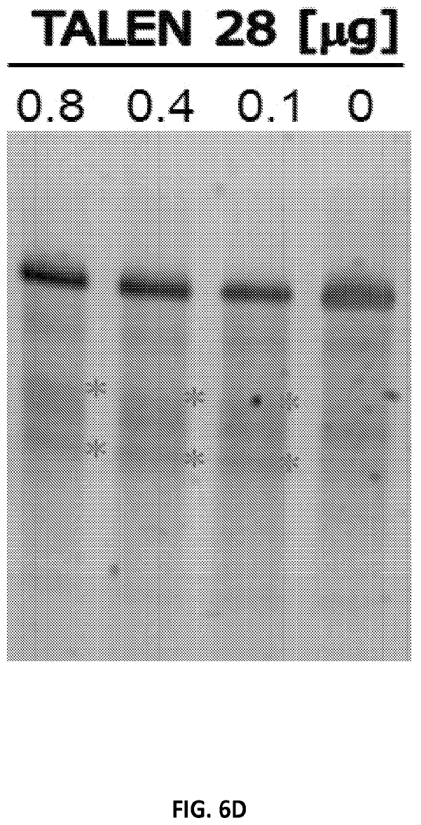

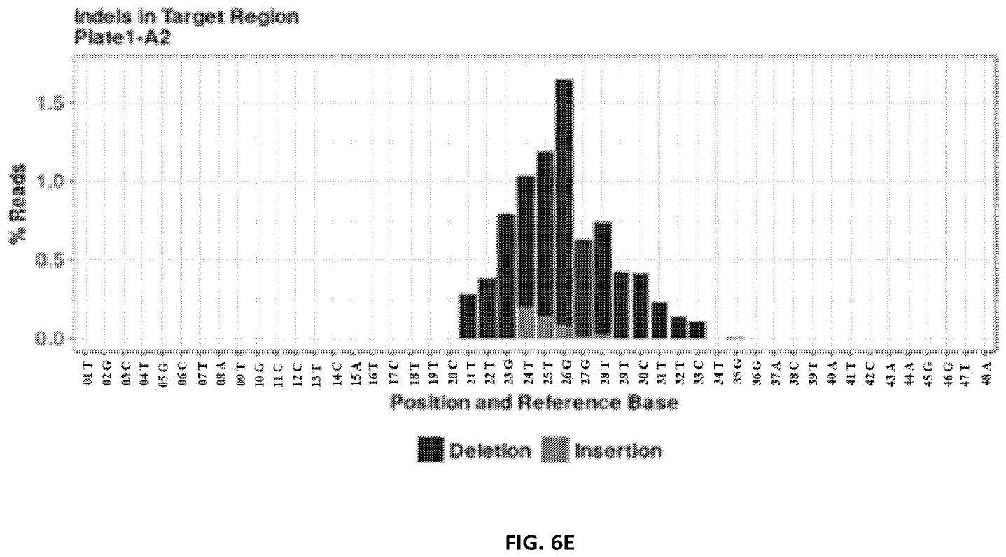

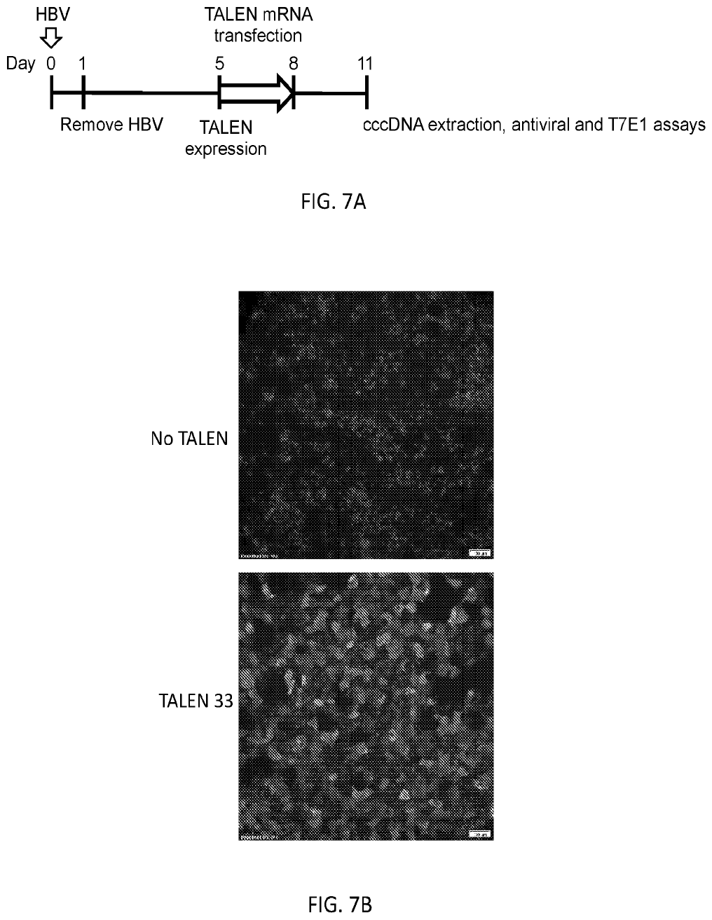

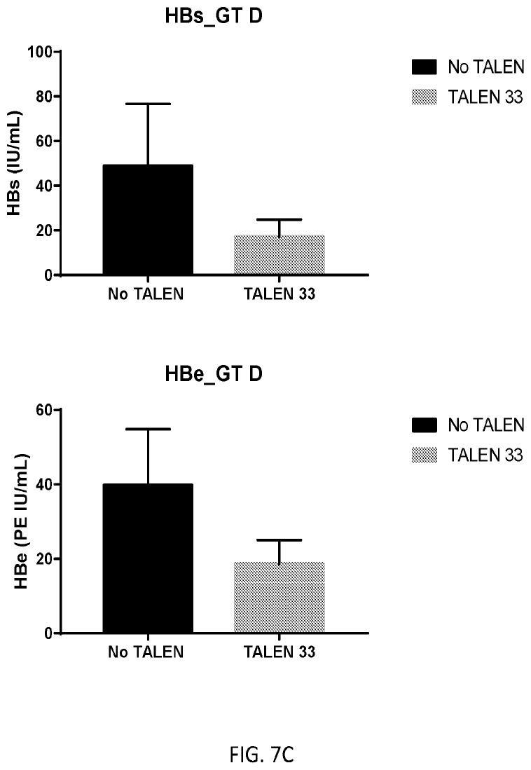

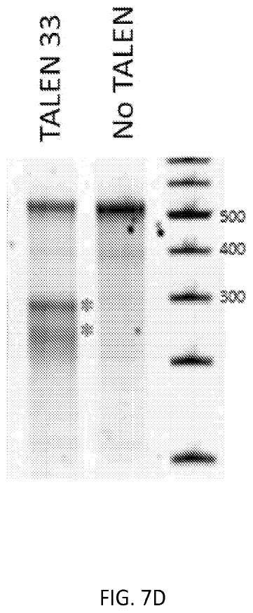

Primary human hepatocytes were infected for 5 days before being transfected with TALEN 28 or TALEN 33 mRNA. After changing the media 3 days post-transfection, HBV cccDNA and cell culture media was collected for gene editing and antiviral assays (FIG. 6A and FIG. 7A for TALEN 28 and TALEN 33, respectively). TALEN 28 expression was detected by immunofluorescence 24 hours after transfection of different mRNA amounts (FIG. 6C). A strong reduction of HBsAg and HBeAg levels in the cell culture media was detected 6 days post-transfection of TALEN 28 mRNA compared to the levels in untransfected cells (FIG. 6C). HBV DNA cleavage activity was analyzed in the HBV cccDNA extracted from the same treated and untreated cells by T7E1 digestion assay (FIG. 6D). As shown in FIG. 6D, DNA corresponding to edited HBV DNA could be detected in transfected cells. By contrast, edited DNA was absent in untransfected cells (FIG. 6D). To confirm the nature of the HBV cccDNA modifications introduced by TALEN 28, the targeted HBV DNA region was sequenced by NGS. Most of the modifications detected corresponded to deletions in the predicted FokI cleavage site (FIG. 6E), confirming the efficacy of TALEN 28 modifying HBV cccDNA.

The same analysis was performed for TALEN 33 (FIG. 7A). First, TALEN protein expression in PHH was confirmed by immunofluorescence staining 6 hours after mRNA transfection (FIG. 7B). TALEN 33 efficacy was evaluated in infected cells 6 days post transfection (11 days post infection with HBV genotype D). TALEN 33 mRNA transfection reduced the levels both of HBsAg and HBeAg in the cell culture media of treated PHH (FIG. 7C). After extraction of HBV cccDNA from the same PHH samples, T7 endonuclease1 digestion assay confirmed the presence of modified HBV DNA in cells transfected with TALEN 33 mRNA (FIG. 7D). Furthermore, TALEN 33 mRNA transfection was able to disrupt HBV cccDNA from PHH cells infected with HBV of A, B, C or D genotypes, as shown by T7E1 digestion assay (FIG. 8) and also gene editing quantification by NGS (Table 3). These results confirm the ability of TALEN mRNA transfection to reduce secretion of HBV viral markers into the cell culture media and to modify HBV DNA of different genotypes by gene editing.

### Example 5: LNP-TALEN Reduces HBV Markers In Vivo in an AAV HBV Mouse Model

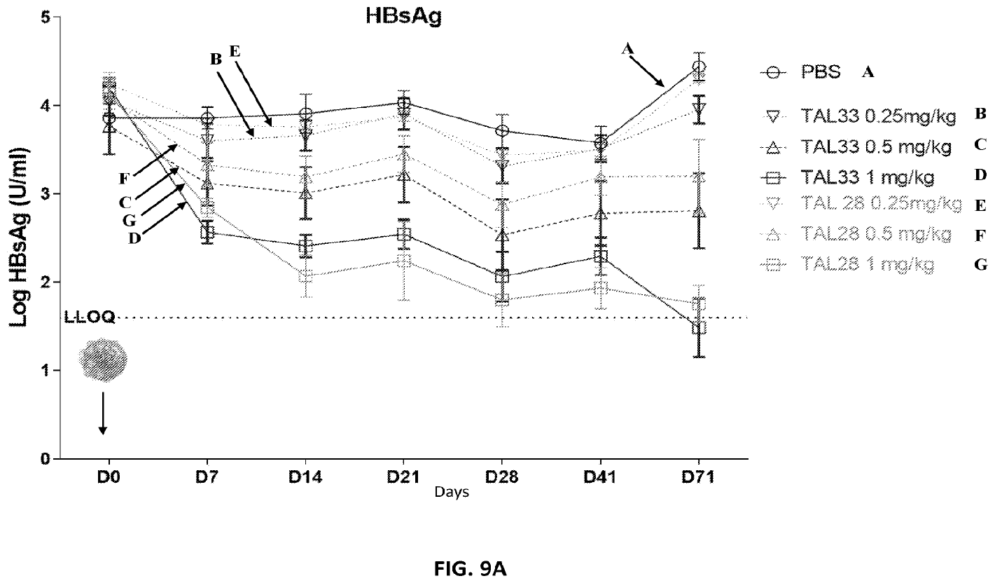

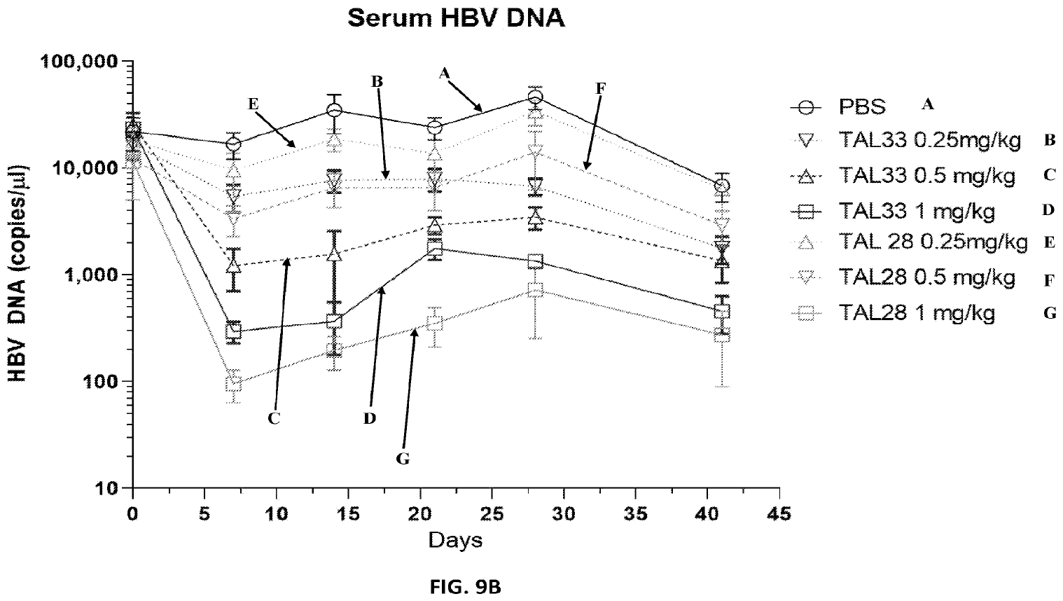

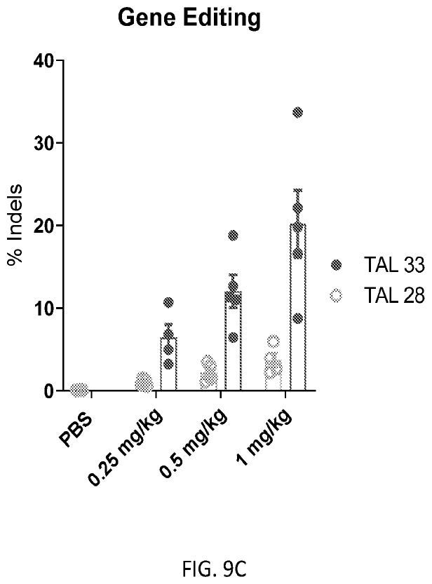

To evaluate the efficacy of TALEN mRNA treatment in vivo, AAV-HBV mice were injected with different concentrations of TALEN 28 and TALEN 33 mRNA formulated in lipid nanoparticles (LNP) or PBS as a negative control. After a single dose at Day 0 (DO), serum levels of HBsAg dropped significantly from day 7 post-dosing in mice injected with 0.5 mg/kg and 1 mg/kg and to a lesser degree in mice injected with 0.25 mg/kg (FIG. 9A). The reduction of HBsAg was maintained until the last time point evaluated, day 71 post-dosing. At the highest dose (1 mg/kg) of LNP-TALEN 28 and 33, the reduction of HBsAg reached 2 logs of HBsAg, close to the lower limit of quantification of the CLIA system used for quantification. Parallel to the reduction of HBsAg levels, HBV DNA levels in the blood serum were also significantly reduced by day 7 post-treatment at all doses tested, i.e., at doses of 0.25 mg/kg, 0.5 mg/kg and 1 mg/kg, up to day 71 (FIG. 9B). The reduction of HBsAg and HBV DNA markers in the serum was maintained until day 71 post dosing, consistent with an irreversible reduction. To verify the HBV DNA modification activity of TALEN proteins in treated AAV-HBV mice, total DNA was extracted from liver tissues and the HBV TALEN target sites were sequenced by NGS. Analysis of NGS data showed that TALEN 28 and TALEN 33 induced indel modifications in the HBV DNA target sequence and that the amount of modified DNA detected increased with increasing LNP-TALEN doses (FIG. 9C). Without being limited by theory, because HBV DNA modifications induced by TALEN are irreversible, the possibility of a rebound in serum levels of HBsAg or HBV DNA is reduced.

### Example 6: Reduction of HBV Serum Markers In Vivo after Single LNP-TALEN Treatment is Irreversible Compared with Nucleoside Inhibitors

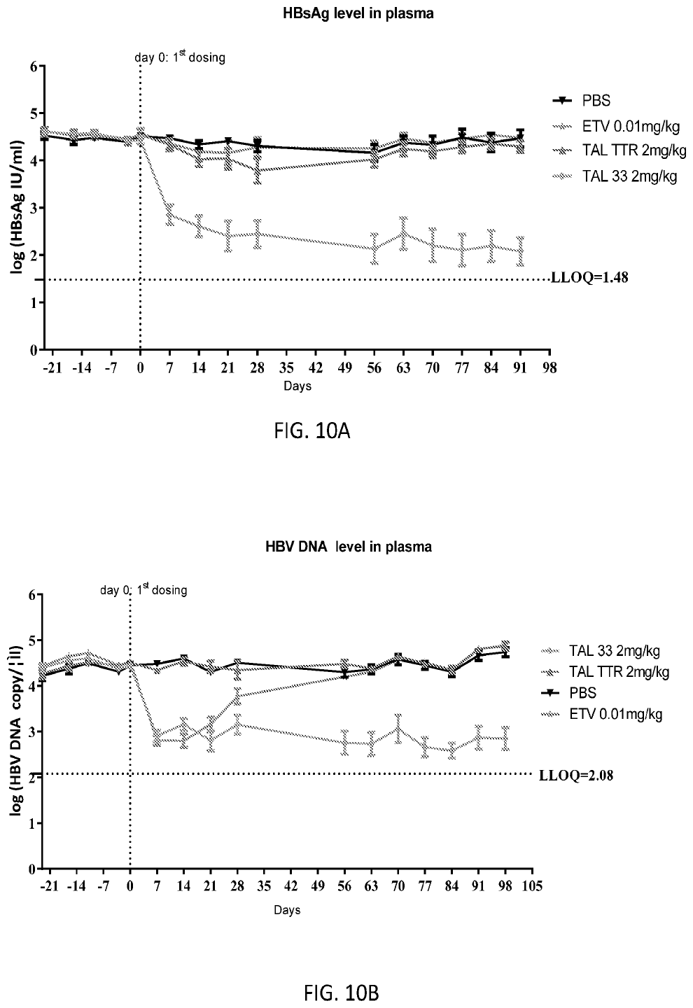

To compare nucleoside inhibitor therapeutics with LNP-TALEN treatment, the AAV-HBV mouse model was used. Mice with serum levels above 4 logs of HBsAg and HBV DNA were selected and treated with either the nucleoside inhibitor entecavir (ETV, 0.01 mg/kg), administered daily for seven days (QD), the HBV-targeting LNP-TALEN 33, administered as a single IV injection of 2 mg/kg, or a non-HBV targeting TALEN TTR, administered as a single IV injection of 2 mg/kg. Single injection of LNP-TALEN 33 reduced plasma HBsAg (FIG. 10A) and HBV DNA (FIG. 10B) levels permanently for over 91 days. No effect on HBV serum markers was detected with non-HBV targeting LNP-TALEN TTR treatment (FIG. 10A, 10B). ETV treatment only reduced plasma HBV DNA levels but not HBsAg levels during the treatment period (FIGS. 10A and 10B), with a viral rebound seen once the treatment was ended (day 7) (FIG. 10B).

SEQUENCE LISTING

| SEQ | | |
|---|---|---|
| ID NO: | Name | SEQUENCE |
| 1 | TALE 28 Left | TGCTGCTATGCCTCATCTTC |
| | DNA binding | |
| | target sequence | |
| 2 | TALE 28 Right | CTGGACTATCAAGGTA |
| | DNA binding | |
| | target sequence | |
| 3 | TALE 33 Left | TCCTGCTGCTATGCCTCAT |
| | DNA binding | |
| | target sequence | |
| 4 | TALE 33 Right | CTTCTGGACTATCAAGGTA |
| | DNA binding | |
| | target sequence | |
| 5 | TALE 28 DNA | TGCTGCTATGCCTCATCTTCTTGTTGGTTCTTCTGGACTATCAAGGTA |
| | binding target | |
| | sequence | |
| 6 | TALE 33 DNA | TCCTGCTGCTATGCCTCATCTTCTTATTGGTTCTTCTGGACTATCAAGGTA |
| | binding target | |
| | sequence | |
| 7 | TALE 28 Left | LTPEQVVAIASNNGGKQALETVQRLLPVLCQAHGLTPEQVVAIASHDGGKQALETVQRLLPVLCQAHGLTPEQVVAI |
| | DNA binding | ASNGGGKQALETVQRLLPVLCQAHGLTPEQVVAIASNNGGKQALETVQRLLPVLCQAHGLTPEQVVAIASHDGGKQ |
| | sequence | ALETVQRLLPVLCQAHGLTPEQVVAIASNGGGKQALETVQRLLPVLCQAHGLTPEQVVAIASNIGGKQALETVQRLL |
| | | PVLCQAHGLTPEQVVAIASNGGGKQALETVQRLLPVLCQAHGLTPEQVVAIASNNGGKQALETVQRLLPVLCQAHG |
| | | LTPEQVVAIASHDGGKQALETVQRLLPVLCQAHGLTPEQVVAIASHDGGKQALETVQRLLPVLCQAHGLTPEQVVAI |
| | | ASNGGGKQALETVQRLLPVLCQAHGLTPEQVVAIASHDGGKQALETVQRLLPVLCQAHGLTPEQVVAIASNIGGKQ |
| | | ALETVQRLLPVLCQAHGLTPEQVVAIASNGGGKQALETVQRLLPVLCQAHGLTPEQVVAIASHDGGKQALETVQRL |
| | | LPVLCQAHGLTPEQVVAIASNGGGKQALETVQRLLPVLCQAHGLTPEQVVAIASNGGGKQALETVQRLLPVLCQAH |
| | | GLTPEQVVAIASHDGG |
| 8 | TALE 28 Right | LTPEQVVAIASNIGGKQALETVQRLLPVLCQAHGLTPEQVVAIASHDGGKQALETVQRLLPVLCQAHGLTPEQVVAI |
| | DNA binding | ASHDGGKQALETVQRLLPVLCQAHGLTPEQVVAIASNGGGKQALETVQRLLPVLCQAHGLTPEQVVAIASNGGGKQ |
| | sequence | ALETVQRLLPVLCQAHGLTPEQVVAIASNNGGKQALETVQRLLPVLCQAHGLTPEQVVAIASNIGGKQALETVQRLL |
| | | PVLCQAHGLTPEQVVAIASNGGGKQALETVQRLLPVLCQAHGLTPEQVVAIASNIGGKQALETVQRLLPVLCQAHGL |
| | | TPEQVVAIASNNGGKQALETVQRLLPVLCQAHGLTPEQVVAIASNGGGKQALETVQRLLPVLCQAHGLTPEQVVAIA |
| | | SHDGGKQALETVQRLLPVLCQAHGLTPEQVVAIASHDGGKQALETVQRLLPVLCQAHGLTPEQVVAIASNIGGKQA |
| | | LETVQRLLPVLCQAHGLTPEQVVAIASNNGG |
| 9 | TALE 33 Left | LTPEQVVAIASHDGGKQALETVQRLLPVLCQAHGLTPEQVVAIASHDGGKQALETVQRLLPVLCQAHGLTPEQVVAI |
| | DNA binding | ASNGGGKQALETVQRLLPVLCQAHGLTPEQVVAIASNNGGKQALETVQRLLPVLCQAHGLTPEQVVAIASHDGGKQ |
| | sequence | ALETVQRLLPVLCQAHGLTPEQVVAIASNGGGKQALETVQRLLPVLCQAHGLTPEQVVAIASNNGGKQALETVQRL |
| | | LPVLCQAHGLTPEQVVAIASHDGGKQALETVQRLLPVLCQAHGLTPEQVVAIASNGGGKQALETVQRLLPVLCQAH |
| | | GLTPEQVVAIASNIGGKQALETVQRLLPVLCQAHGLTPEQVVAIASNGGGKQALETVQRLLPVLCQAHGLTPEQVVA |
| | | IASNNGGKQALETVQRLLPVLCQAHGLTPEQVVAIASHDGGKQALETVQRLLPVLCQAHGLTPEQVVAIASHDGGK |
| | | QALETVQRLLPVLCQAHGLTPEQVVAIASNGGGKQALETVQRLLPVLCQAHGLTPEQVVAIASHDGGKQALETVQR |
| | | LLPVLCQAHGLTPEQVVAIASNIGGKQALETVQRLLPVLCQAHGLTPEQVVAIASNGGG |
| 10 | TALE 33 Right | LTPEQVVAIASNIGGKQALETVQRLLPVLCQAHGLTPEQVVAIASHDGGKQALETVQRLLPVLCQAHGLTPEQVVAI |
| | DNA binding | ASHDGGKQALETVQRLLPVLCQAHGLTPEQVVAIASNGGGKQALETVQRLLPVLCQAHGLTPEQVVAIASNGGGKQ |
| | sequence | ALETVQRLLPVLCQAHGLTPEQVVAIASNNGGKQALETVQRLLPVLCQAHGLTPEQVVAIASNIGGKQALETVQRLL |
| | | PVLCQAHGLTPEQVVAIASNGGGKQALETVQRLLPVLCQAHGLTPEQVVAIASNIGGKQALETVQRLLPVLCQAHGL |
| | | TPEQVVAIASNNGGKQALETVQRLLPVLCQAHGLTPEQVVAIASNGGGKQALETVQRLLPVLCQAHGLTPEQVVAIA |
| | | SHDGGKQALETVQRLLPVLCQAHGLTPEQVVAIASHDGGKQALETVQRLLPVLCQAHGLTPEQVVAIASNIGGKQA |
| | | LETVQRLLPVLCQAHGLTPEQVVAIASNNGGKQALETVQRLLPVLCQAHGLTPEQVVAIASNIGGKQALETVQRLLP |
| | | VLCQAHGLTPEQVVAIASNIGGKQALETVQRLLPVLCQAHGLTPEQVVAIASNNGG |
| 11 | FokI Nuclease | QLVKSELEEKKSELRHKLKYVPHEYIELIEIARNSTQDRILEMKVMEFFMKVYGYRGKHLGGSRKPDGAIYTVGSPID |
| | | YGVIVDTKAYSGGYNLPIGQADEMQRYVEENQTRNKHINPNEWWKVYPSSVTEFKFLFVSGHFKGNYKAQLTRLN |
| | | HITNCNGAVLSVEELLIGGEMIKAGTLTLEEVRRKFNNGEINFRS |
| 12 | TEV 5′UTR | UCAACACAACAUAUACAAAACAAACGAAUCUCAAGCAAUCAAGCAUUCUACUUCUAUUGCAGCAAUUUAA |
| | | AUCAUUUCUUUUAAAGCAAAAGCAAUUUUCUGAAAAUUUUCACCAUUUACGAACGAUAG |
| 13 | AT1G58420 | AUUAUUACAUCAAAACAAAAAGCCGCCA |
| | 5′UTR | |
| 14 | ARC5-2 5′UTR | CUUAAGGGGGCGCUGCCUACGGAGGUGGCAGCCAUCUCCUUCUCGGCAUCAAGCUUACCAUGGUGCCCCAG |
| | | GCCCUGCUCUUGGUCCCGCUGCUGGUGUUCCCCCUCUGCUUCGGCAAGUUCCCCAUCUACACCAUCCCCGA |
| | | CAAGCUGGGGCCGUGGAGCCCCAUCGACAUCCACCACCUGUCCUGCCCCAACAACCUCGUGGUCGAGGACG |
| | | AGGGCUGCACCAACCUGAGCGGGUUCUCCUAC |
| 15 | HCV 5′UTR | UGAGUGUCGUACAGCCUCCAGGCCCCCCCCUCCCGGGAGAGCCAUAGUGGUCUGCGGAACCGGUGAGUACA |
| | | CCGGAAUUGCCGGGAAGACUGGGUCCUUUCUUGGAUAAACCCACUCUAUGCCCGGCCAUUUGGGCGUGCCC |
| | | CCGCAAGACUGCUAGCCGAGUAGUGUUGGGUUGCG |
| 16 | Human Albumin | AAUUAUUGGUUAAAGAAGUAUAUUAGUGCUAAUUUCCCUCCGUUUGUCCUAGCUUUUCUCUUCUGUCAAC |
| | 5′UTR | CCCACACGCCUUUGGCACA |
| 17 | EMCV 5′UTR | CUCCCUCCCCCCCCCCUAACGUUACUGGCCGAAGCCGCUUGGAAUAAGGCCGGUGUGCGUUUGUCUAUAUG |
| | | UUAUUUUCCACCAUAUUGCCGUCUUUUGGCAAUGUGAGGGCCCGGAAACCUGGCCCUGUCUUCUUGACGA |
| | | GCAUUCCUAGGGGUCUUUCCCCUCUCGCCAAAGGAAUGCAAGGUCUGUUGAAUGUCGUGAAGGAAGCAGU |
| | | UCCUCUGGAAGCUUCUUGAAGACAAACAACGUCUGUAGCGACCCUUUGCAGGCAGCGGAACCCCCCACCUG |
| | | GCGACAGGUGCCUCUGCGGCCAAAAGCCACGUGUAUAAGAUACACCUGCAAAGGCGGCACAACCCCAGUGC |
| | | CACGUUGUGAGUUGGAUAGUUGUGGAAAGAGUCAAAUGGCUCUCCUCAAGCGUAUUCAACAAGGGGCUGA |
| | | AGGAUGCCCAGAAGGUACCCCAUUGUAUGGGAUCUGAUCUGGGGCCUCGGUGCACAUGCUUUACGUGUGU |
| | | UUAGUCGAGGUUAAAAAACGUCUAGGCCCCCCGAACCACGGGGACGUGGUUUUCCUUUGAAAAACACGAU |
| | | GAUAAU |
| 18 | XBG 3′UTR | CUAGUGACUGACUAGGAUCUGGUUACCACUAAACCAGCCUCAAGAACACCCGAAUGGAGUCUCUAAGCUA |
| | | CAUAAUACCAACUUACACUUACAAAAUGUUGUCCCCCAAAAUGUAGCCAUUCGUAUCUGCUCCUAAUAAA |
| | | AAGAAAGUUUCUUCACAU |
| 19 | Human | UGCAAGGCUGGCCGGAAGCCCUUGCCUGAAAGCAAGAUUUCAGCCUGGAAGAGGGCAAAGUGGACGGGAG |
| | haptoglobin | UGGACAGGAGUGGAUGCGAUAAGAUGUGGUUUGAAGCUGAUGGGUGCCAGCCCUGCAUUGCUGAGUCAAU |
| | 3′UTR | CAAUAAAGAGCUUUCUUUUGACCCAU |
| 20 | Human | ACGCCGAAGCCUGCAGCCAUGCGACCCCACGCCACCCCGUGCCUCCUGCCUCCGCGCAGCCUGCAGCGGGAG |
| | Apolipoprotein E | ACCCUGUCCCCGCCCCAGCCGUCCUCCUGGGGUGGACCCUAGUUUAAUAAAGAUUCACCAAGUUUCACGCA |
| | 3′UTR | |
| 21 | HCV 3′UTR | UAGAGCGGCAAACCCUAGCUACACUCCAUAGCUAGUUUCUUUUUUUUUUGUUUUUUUUUUUUUUUUUUUU |
| | | UUUUUUUUUUUUUUUUUUUCCUUUCUUUUCCUUUUUUUUUCCUCUUUUCUUGGUGGCUCCAUCUUAGCC |
| | | CUAGUCACGGCUAGCUGUGAAAGGUCCGUGAGCCGCAUGACUGCAGAGAGUGCCGUAACUGGUCUCUCUG |
| | | CAGAUCAUGU |
| 22 | Mouse albumin | ACACAUCACAACCACAACCUUCUCAGGCUACCCUGAGAAAAAAAGACAUGAAGACUCAGGACUCAUCUUUU |
| | 3′UTR | CUGUUGGUGUAAAAUCAACACCCUAAGGAACACAAAUUUCUUUAAACAUUUGACUUCUUGUCUCUGUGCU |
| | | GCAAUUAAUAAAAAAUGGAAAGAAUCUAC |
| 23 | Human alpha | GCUGGAGCCUCGGUAGCCGUUCCUCCUGCCCGCUGGGCCUCCCAACGGGCCCUCCUCCCCUCCUUGCACCGG |
| | globin 3′UTR | CCCUUCCUGGUCUUUGAAUAAAGUCUGAGUGGGCAGCA |
| 24 | EMCV 3′UTR | UAGUGCAGUCACUGGCACAACGCGUUGCCCGGUAAGCCAAUCGGGUAUACACGGUCGUCAUACUGCAGAC |
| | | AGGGUUCUUCUACUUUGCAAGAUAGUCUAGAGUAGUAAAAUAAAUAGUAUAAG |
| 25 | TALEN 28 Left | MDYKDHDGDYKDHDIDYKDDDDKGGSGPKKKRKVGIHGVPAAVDLRTLGYSQQQQEKIKPKVRSTVAQHHEAL |
| | protein | VGHGFTHAHIVALSQHPAALGTVAVKYQDMIAALPEATHEAIVGVGKQWSGARALEALLTVAGELRGPPLQLDT |
| | | GQLLKIAKRGGVTAVEAVHAWRNALTGAPLNLTPEQVVAIASNNGGKQALETVQRLLPVLCQAHGLTPEQVVAI |
| | | ASHDGGKQALETVQRLLPVLCQAHGLTPEQVVAIASNGGGKQALETVQRLLPVLCQAHGLTPEQVVAIASNNGGK |
| | | QALETVQRLLPVLCQAHGLTPEQVVAIASHDGGKQALETVQRLLPVLCQAHGLTPEQVVAIASNGGGKQALETVQ |
| | | RLLPVLCQAHGLTPEQVVAIASNIGGKQALETVQRLLPVLCQAHGLTPEQVVAIASNGGGKQALETVQRLLPVLCQ |
| | | AHGLTPEQVVAIASNNGGKQALETVQRLLPVLCQAHGLTPEQVVAIASHDGGKQALETVQRLLPVLCQAHGLTPE |
| | | QVVAIASHDGGKQALETVQRLLPVLCQAHGLTPEQVVAIASNGGGKQALETVQRLLPVLCQAHGLTPEQVVAIAS |
| | | HDGGKQALETVQRLLPVLCQAHGLTPEQVVAIASNIGGKQALETVQRLLPVLCQAHGLTPEQVVAIASNGGGKQA |
| | | LETVQRLLPVLCQAHGLTPEQVVAIASHDGGKQALETVQRLLPVLCQAHGLTPEQVVAIASNGGGKQALETVQRL |
| | | LPVLCQAHGLTPEQVVAIASNGGGKQALETVQRLLPVLCQAHGLTPEQVVAIASHDGGRPALESIVAQLSRPDPAL |
| | | AALTNDHLVALACLGGRPALDAVKKGLPHAPALIKRTNRRIPERTSHRVAGSQLVKSELEEKKSELRHKLKYVPHE |
| | | YIELIEIARNSTQDRILEMKVMEFFMKVYGYRGKHLGGSRKPDGAIYTVGSPIDYGVIVDTKAYSGGYNLPIGQADE |
| | | MQRYVEENQTRNKHINPNEWWKVYPSSVTEFKFLFVSGHFKGNYKAQLTRLNHITNCNGAVLSVEELLIGGEMIK |
| | | AGTLTLEEVRRKENNGEINFRS |
| 26 | TALEN 28 Right | MDYKDHDGDYKDHDIDYKDDDDKGGSGPKKKRKVGIHGVPAAVDLRTLGYSQQQQEKIKPKVRSTVAQHHEAL |
| | protein | VGHGFTHAHIVALSQHPAALGTVAVKYQDMIAALPEATHEAIVGVGKQWSGARALEALLTVAGELRGPPLQLDT |
| | | GQLLKIAKRGGVTAVEAVHAWRNALTGAPLNLTPEQVVAIASNIGGKQALETVQRLLPVLCQAHGLTPEQVVAIA |
| | | SHDGGKQALETVQRLLPVLCQAHGLTPEQVVAIASHDGGKQALETVQRLLPVLCQAHGLTPEQVVAIASNGGGKQ |
| | | ALETVQRLLPVLCQAHGLTPEQVVAIASNGGGKQALETVQRLLPVLCQAHGLTPEQVVAIASNNGGKQALETVQR |
| | | LLPVLCQAHGLTPEQVVAIASNIGGKQALETVQRLLPVLCQAHGLTPEQVVAIASNGGGKQALETVQRLLPVLCQA |
| | | HGLTPEQVVAIASNIGGKQALETVQRLLPVLCQAHGLTPEQVVAIASNNGGKQALETVQRLLPVLCQAHGLTPEQV |
| | | VAIASNGGGKQALETVQRLLPVLCQAHGLTPEQVVAIASHDGGKQALETVQRLLPVLCQAHGLTPEQVVAIASHD |
| | | GGKQALETVQRLLPVLCQAHGLTPEQVVAIASNIGGKQALETVQRLLPVLCQAHGLTPEQVVAIASNNGGRPALES |
| | | IVAQLSRPDPALAALTNDHLVALACLGGRPALDAVKKGLPHAPALIKRTNRRIPERTSHRVAGSQLVKSELEEKKS |
| | | ELRHKLKYVPHEYIELIEIARNSTQDRILEMKVMEFFMKVYGYRGKHLGGSRKPDGAIYTVGSPIDYGVIVDTKAY |
| | | SGGYNLPIGQADEMQRYVEENQTRNKHINPNEWWKVYPSSVTEFKFLFVSGHFKGNYKAQLTRLNHITNCNGAVL |
| | | SVEELLIGGEMIKAGTLTLEEVRRKFNNGEINFRS |
| 27 | TALEN 33 Left | MDYKDHDGDYKDHDIDYKDDDDKGGSGPKKKRKVGIHGVPAAVDLRTLGYSQQQQEKIKPKVRSTVAQHHEAL |
| | protein | VGHGFTHAHIVALSQHPAALGTVAVKYQDMIAALPEATHEAIVGVGKQWSGARALEALLTVAGELRGPPLQLDT |
| | | GQLLKIAKRGGVTAVEAVHAWRNALTGAPLNLTPEQVVAIASHDGGKQALETVQRLLPVLCQAHGLTPEQVVAI |
| | | ASHDGGKQALETVQRLLPVLCQAHGLTPEQVVAIASNGGGKQALETVQRLLPVLCQAHGLTPEQVVAIASNNGGK |
| | | QALETVQRLLPVLCQAHGLTPEQVVAIASHDGGKQALETVQRLLPVLCQAHGLTPEQVVAIASNGGGKQALETVQ |
| | | RLLPVLCQAHGLTPEQVVAIASNNGGKQALETVQRLLPVLCQAHGLTPEQVVAIASHDGGKQALETVQRLLPVLC |
| | | QAHGLTPEQVVAIASNGGGKQALETVQRLLPVLCQAHGLTPEQVVAIASNIGGKQALETVQRLLPVLCQAHGLTPE |
| | | QVVAIASNGGGKQALETVQRLLPVLCQAHGLTPEQVVAIASNNGGKQALETVQRLLPVLCQAHGLTPEQVVAIAS |
| | | HDGGKQALETVQRLLPVLCQAHGLTPEQVVAIASHDGGKQALETVQRLLPVLCQAHGLTPEQVVAIASNGGGKQ |
| | | ALETVQRLLPVLCQAHGLTPEQVVAIASHDGGKQALETVQRLLPVLCQAHGLTPEQVVAIASNIGGKQALETVQRL |
| | | LPVLCQAHGLTPEQVVAIASNGGGRPALESIVAQLSRPDPALAALTNDHLVALACLGGRPALDAVKKGLPHAPALI |
| | | KRTNRRIPERTSHRVAGSQLVKSELEEKKSELRHKLKYVPHEYIELIEIARNSTQDRILEMKVMEFFMKVYGYRGK |
| | | HLGGSRKPDGAIYTVGSPIDYGVIVDTKAYSGGYNLPIGQADEMQRYVEENQTRNKHINPNEWWKVYPSSVTEFK |
| | | FLFVSGHFKGNYKAQLTRLNHITNCNGAVLSVEELLIGGEMIKAGTLTLEEVRRKFNNGEINFRS |
| 28 | TALEN 33 Right | MDYKDHDGDYKDHDIDYKDDDDKGGSGPKKKRKVGIHGVPAAVDLRTLGYSQQQQEKIKPKVRSTVAQHHEAL |
| | protein | VGHGFTHAHIVALSQHPAALGTVAVKYQDMIAALPEATHEAIVGVGKQWSGARALEALLTVAGELRGPPLQLDT |
| | | GQLLKIAKRGGVTAVEAVHAWRNALTGAPLNLTPEQVVAIASNIGGKQALETVQRLLPVLCQAHGLTPEQVVAIA |
| | | SHDGGKQALETVQRLLPVLCQAHGLTPEQVVAIASHDGGKQALETVQRLLPVLCQAHGLTPEQVVAIASNGGGKQ |
| | | ALETVQRLLPVLCQAHGLTPEQVVAIASNGGGKQALETVQRLLPVLCQAHGLTPEQVVAIASNNGGKQALETVQR |
| | | LLPVLCQAHGLTPEQVVAIASNIGGKQALETVQRLLPVLCQAHGLTPEQVVAIASNGGGKQALETVQRLLPVLCQA |
| | | HGLTPEQVVAIASNIGGKQALETVQRLLPVLCQAHGLTPEQVVAIASNNGGKQALETVQRLLPVLCQAHGLTPEQV |
| | | VAIASNGGGKQALETVQRLLPVLCQAHGLTPEQVVAIASHDGGKQALETVQRLLPVLCQAHGLTPEQVVAIASHD |
| | | GGKQALETVQRLLPVLCQAHGLTPEQVVAIASNIGGKQALETVQRLLPVLCQAHGLTPEQVVAIASNNGGKQALE |
| | | TVQRLLPVLCQAHGLTPEQVVAIASNIGGKQALETVQRLLPVLCQAHGLTPEQVVAIASNIGGKQALETVQRLLPV |
| | | LCQAHGLTPEQVVAIASNNGGRPALESIVAQLSRPDPALAALTNDHLVALACLGGRPALDAVKKGLPHAPALIKRT |
| | | NRRIPERTSHRVAGSQLVKSELEEKKSELRHKLKYVPHEYIELIEIARNSTQDRILEMKVMEFFMKVYGYRGKHLG |
| | | GSRKPDGAIYTVGSPIDYGVIVDTKAYSGGYNLPIGQADEMQRYVEENQTRNKHINPNEWWKVYPSSVTEFKFLFV |
| | | SGHFKGNYKAQLTRLNHITNCNGAVLSVEELLIGGEMIKAGTLTLEEVRRKFNNGEINFRS |
| 29 | Forward primer | CCTAGGACCCCTTCTCGTGT |
| 30 | Reverse primer | ACTGAGCCAGGAGAAACGGG |
| 31 | SV40 Large T- | PKKKRKV |
| | Antigen NLS | |
| 32 | nucleoplasmin | KRPAATKKAGQAKKKK |
| | NLS | |
| 33 | EGL-13 NLS | MSRRRKANPTKLSENAKKLAKEVEN |
| 34 | c-Myc proto- | PAAKRVKLD |
| | oncoprotein NLS | |
| 35 | TUS-protein NLS | KLKIKRPVK |
| 36 | FLAG tag | DYKDDDDK |
| 37 | FLAG tag | EYKEEEEK |
| 38 | hexa-histidine | HHHHHH |
| 39 | myc tag | EQKLISEEDL |
| 40 | influenza HA tag | YPYDVPDYA |
| 41 | AcV5 tag | SWKDASGWS |
| 42 | ALFA-tag | SRLEEELRRRLTE |
| 43 | AviTag | GLNDIFEAQKIEWHE |
| 44 | E-tag | GAPVPYPDPLEPR |
| 45 | S-tag | KETAAAKFERQHMDS |
| 46 | Strep-tag | WSHPQFEK |
| 47 | T7 tag | MASMTGGQQMG |
| 48 | Ty 1 tag | EVHTNQDPLD |
| 49 | V5 tag | GKPIPNPLLGLDST |
| 50 | VSV-G tag | YTDIEMNRLGK |
| 51 | Xpress tag | DLYDDDDK |
| 52 | TALE 28 Left | VDLRTLGYSQQQQEKIKPKVRSTVAQHHEALVGHGFTHAHIVALSQHPAALGTVAVKYQDMIAALPEATHEAIVG |
| | DNA binding | VGKQWSGARALEALLTVAGELRGPPLQLDTGQLLKIAKRGGVTAVEAVHAWRNALTGAPLNLTPEQVVAIASNN |
| | sequence with N- | GGKQALETVQRLLPVLCQAHGLTPEQVVAIASHDGGKQALETVQRLLPVLCQAHGLTPEQVVAIASNGGGKQALE |
| | terminal | TVQRLLPVLCQAHGLTPEQVVAIASNNGGKQALETVQRLLPVLCQAHGLTPEQVVAIASHDGGKQALETVQRLLP |
| | | VLCQAHGLTPEQVVAIASNGGGKQALETVQRLLPVLCQAHGLTPEQVVAIASNIGGKQALETVQRLLPVLCQAHG |
| | | LTPEQVVAIASNGGGKQALETVQRLLPVLCQAHGLTPEQVVAIASNNGGKQALETVQRLLPVLCQAHGLTPEQVV |
| | | AIASHDGGKQALETVQRLLPVLCQAHGLTPEQVVAIASHDGGKQALETVQRLLPVLCQAHGLTPEQVVAIASNGG |
| | | GKQALETVQRLLPVLCQAHGLTPEQVVAIASHDGGKQALETVQRLLPVLCQAHGLTPEQVVAIASNIGGKQALET |
| | | VQRLLPVLCQAHGLTPEQVVAIASNGGGKQALETVQRLLPVLCQAHGLTPEQVVAIASHDGGKQALETVQRLLPV |
| | | LCQAHGLTPEQVVAIASNGGGKQALETVQRLLPVLCQAHGLTPEQVVAIASNGGGKQALETVQRLLPVLCQAHGL |
| | | TPEQVVAIASHDGG |
| 53 | TALE 28 Right | VDLRTLGYSQQQQEKIKPKVRSTVAQHHEALVGHGFTHAHIVALSQHPAALGTVAVKYQDMIAALPEATHEAIVG |
| | DNA binding | VGKQWSGARALEALLTVAGELRGPPLQLDTGQLLKIAKRGGVTAVEAVHAWRNALTGAPLNLTPEQVVAIASNIG |
| | sequence with N- | GKQALETVQRLLPVLCQAHGLTPEQVVAIASHDGGKQALETVQRLLPVLCQAHGLTPEQVVAIASHDGGKQALET |
| | terminal | VQRLLPVLCQAHGLTPEQVVAIASNGGGKQALETVQRLLPVLCQAHGLTPEQVVAIASNGGGKQALETVQRLLPV |
| | | LCQAHGLTPEQVVAIASNNGGKQALETVQRLLPVLCQAHGLTPEQVVAIASNIGGKQALETVQRLLPVLCQAHGL |
| | | TPEQVVAIASNGGGKQALETVQRLLPVLCQAHGLTPEQVVAIASNIGGKQALETVQRLLPVLCQAHGLTPEQVVAI |
| | | ASNNGGKQALETVQRLLPVLCQAHGLTPEQVVAIASNGGGKQALETVQRLLPVLCQAHGLTPEQVVAIASHDGGK |
| | | QALETVQRLLPVLCQAHGLTPEQVVAIASHDGGKQALETVQRLLPVLCQAHGLTPEQVVAIASNIGGKQALETVQ |
| | | RLLPVLCQAHGLTPEQVVAIASNNGG |
| 54 | TALE 33 Left | VDLRTLGYSQQQQEKIKPKVRSTVAQHHEALVGHGFTHAHIVALSQHPAALGTVAVKYQDMIAALPEATHEAIVG |
| | DNA binding | VGKQWSGARALEALLTVAGELRGPPLQLDTGQLLKIAKRGGVTAVEAVHAWRNALTGAPLNLTPEQVVAIASHD |
| | sequence with N- | GGKQALETVQRLLPVLCQAHGLTPEQVVAIASHDGGKQALETVQRLLPVLCQAHGLTPEQVVAIASNGGGKQALE |
| | terminal | TVQRLLPVLCQAHGLTPEQVVAIASNNGGKQALETVQRLLPVLCQAHGLTPEQVVAIASHDGGKQALETVQRLLP |
| | | VLCQAHGLTPEQVVAIASNGGGKQALETVQRLLPVLCQAHGLTPEQVVAIASNNGGKQALETVQRLLPVLCQAHG |
| | | LTPEQVVAIASHDGGKQALETVQRLLPVLCQAHGLTPEQVVAIASNGGGKQALETVQRLLPVLCQAHGLTPEQVV |
| | | AIASNIGGKQALETVQRLLPVLCQAHGLTPEQVVAIASNGGGKQALETVQRLLPVLCQAHGLTPEQVVAIASNNGG |
| | | KQALETVQRLLPVLCQAHGLTPEQVVAIASHDGGKQALETVQRLLPVLCQAHGLTPEQVVAIASHDGGKQALETV |
| | | QRLLPVLCQAHGLTPEQVVAIASNGGGKQALETVQRLLPVLCQAHGLTPEQVVAIASHDGGKQALETVQRLLPVL |
| | | CQAHGLTPEQVVAIASNIGGKQALETVQRLLPVLCQAHGLTPEQVVAIASNGGG |
| 55 | TALE 33 Right | VDLRTLGYSQQQQEKIKPKVRSTVAQHHEALVGHGFTHAHIVALSQHPAALGTVAVKYQDMIAALPEATHEAIVG |
| | DNA binding | VGKQWSGARALEALLTVAGELRGPPLQLDTGQLLKIAKRGGVTAVEAVHAWRNALTGAPLNLTPEQVVAIASNIG |
| | sequence with N- | GKQALETVQRLLPVLCQAHGLTPEQVVAIASHDGGKQALETVQRLLPVLCQAHGLTPEQVVAIASHDGGKQALET |
| | terminal | VQRLLPVLCQAHGLTPEQVVAIASNGGGKQALETVQRLLPVLCQAHGLTPEQVVAIASNGGGKQALETVQRLLPV |
| | | LCQAHGLTPEQVVAIASNNGGKQALETVQRLLPVLCQAHGLTPEQVVAIASNIGGKQALETVQRLLPVLCQAHGL |
| | | TPEQVVAIASNGGGKQALETVQRLLPVLCQAHGLTPEQVVAIASNIGGKQALETVQRLLPVLCQAHGLTPEQVVAI |
| | | ASNNGGKQALETVQRLLPVLCQAHGLTPEQVVAIASNGGGKQALETVQRLLPVLCQAHGLTPEQVVAIASHDGGK |
| | | QALETVQRLLPVLCQAHGLTPEQVVAIASHDGGKQALETVQRLLPVLCQAHGLTPEQVVAIASNIGGKQALETVQ |
| | | RLLPVLCQAHGLTPEQVVAIASNNGGKQALETVQRLLPVLCQAHGLTPEQVVAIASNIGGKQALETVQRLLPVLCQ |
| | | AHGLTPEQVVAIASNIGGKQALETVQRLLPVLCQAHGLTPEQVVAIASNNGG |

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains.

The invention illustratively described herein suitably may be practiced in the absence of any element or elements, limitation or limitations which is not specifically disclosed herein. Thus, for example, in each instance herein any of the terms “comprising”, “consisting essentially of’ and “consisting of’ may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Other embodiments are within the following claims.

We claim:

1. A method for treating hepatitis infection in a subject in need thereof, comprising administering to the subject a combination of mRNA molecules comprising:
(1) a first mRNA molecule comprising a polynucleotide sequence encoding a first transcription activator like effector nuclease (TALEN) monomer comprising a first TALE DNA binding domain and a first nuclease catalytic domain, wherein the first TALE DNA binding domain is capable of binding to a first half-site sequence of a target nucleic acid sequence within an HBV genome; and
(2) a second mRNA molecule comprising a polynucleotide sequence encoding a second TALEN monomer comprising a second TALE DNA binding domain and a second nuclease catalytic domain, wherein the second TALE DNA binding domain is capable of binding to a second half-site sequence of the target nucleic acid sequence;
wherein the first TALEN monomer and the second TALEN monomer are capable of forming a dimer that cleaves the target nucleic acid sequence when the first TALE DNA binding domain binds to the first half-site sequence and the second TALE DNA binding domain binds to the second half-site sequence,
preferably, the first nuclease catalytic domain is a first FokI nuclease catalytic domain and the second nuclease catalytic domain is a second FokI nuclease catalytic domain.

2. The method of claim 1, wherein the target nucleic acid sequence is within the sequence that encodes HBsAg and HBV polymerase (pol), preferably,
(a) the first half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 1, and the second half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 2; or
(b) the first half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 3, and the second half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 4, or
(c) the target nucleic acid sequence comprises the polynucleotide sequence at least 90% identical, such as at least 90%, 91%, 92%, 93%, 94%, 95%, 96%, 97%, 98%, 99% or 100% identical, to the nucleic acid sequence of SEQ ID NO: 5 or SEQ ID NO: 6.

3. The method of claim 1 or 2, wherein the first and second mRNA molecules each further comprise one or more, preferably all, of a 5′ cap, a 5′-UTR, a sequence encoding a nuclear localization signal, a sequence encoding an N-terminal domain, a sequence encoding a C-terminal domain, a 3′-UTR, and a poly adenosine tail.

4. The method of any one of claims 1-3, wherein the first TALE DNA binding domain comprises an amino acid sequence at least 90% identical to SEQ ID NO: 7 or SEQ ID NO: 9, the second TALE DNA binding domain comprises an amino acid sequence at least 90% identical to SEQ ID NO: 8 or SEQ ID NO: 10, respectively, and the first and second FokI nuclease catalytic domain each comprise an amino acid sequence at least 90% identical to SEQ ID NO: 11.

5. The method of any one of claims 1-4, wherein the HBV genome is of one or more of genotypes A, B, C, D, E, F, G, H, I and J.

6. The method of any one of claims 1-5, further comprising administering to the subject a second therapeutic composition, preferably comprising an anti-HBV agent.

7. The method of any one of claims 1-6, wherein the subject has an HBV infection, preferably a chronic HBV infection.

8. The method of any one of claims 1-7, wherein the subject is co-infected with HBV and HDV.

9. A composition comprising a combination of mRNA molecules encapsulated in lipid nanoparticles comprising:
(1) a first mRNA molecule comprising a polynucleotide sequence encoding a first transcription activator like effector nuclease (TALEN) monomer comprising a first TALE DNA binding domain and a first nuclease catalytic domain, wherein the first TALE DNA binding domain is capable of binding to a first half-site sequence of a target nucleic acid sequence within an HBV genome; and
(2) a second mRNA molecule comprising a polynucleotide sequence encoding a second TALEN monomer comprising a second TALE DNA binding domain and a second nuclease catalytic domain, wherein the second TALE DNA binding domain is capable of binding to a second half-site sequence of the target nucleic acid sequence;
wherein the first TALEN monomer and the second TALEN monomer are capable of forming a dimer that cleaves the target nucleic acid sequence when the first TALE DNA binding domain binds to the first half-site sequence and the second TALE DNA binding domain binds to the second half-site sequence,
preferably, the first nuclease catalytic domain is a first FokI nuclease catalytic domain and the second nuclease catalytic domain is a second FokI nuclease catalytic domain.

10. The composition of claim 9, wherein the target nucleic acid sequence is within a sequence that encodes HBsAg and HBV polymerase (pol), preferably,
(a) the first half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 1, and the second half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 2; or
(b) the first half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 3, and the second half-site sequence of the target nucleic acid sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 4, or
(c) the target nucleic acid sequence comprises the polynucleotide sequence at least 90% identical, such as at least 90%, 91%, 92%, 93%, 94%, 95%, 96%, 97%, 98%, 99% or 100% identical, to the nucleic acid sequence of SEQ ID NO: 5 or SEQ ID NO: 6.

11. The composition of claim 9 or claim 10, wherein the first and second mRNA molecules each further comprise one or more, preferably all, of a 5′ cap, a 5′-UTR, a sequence encoding a nuclear localization signal, a sequence encoding an N-terminal domain, a sequence encoding a C-terminal domain, a 3′-UTR, and a poly adenosine tail.

12. The composition of any one of claims 9-11, wherein the first TALE DNA binding domain comprises an amino acid sequence at least 90% identical to SEQ ID NO: 7 or SEQ ID NO: 9, the second TALE DNA binding domain comprises an amino acid sequence at least 90% identical to SEQ ID NO: 8 or SEQ ID NO: 10, respectively, and the first and second FokI nuclease catalytic domain each comprise an amino acid sequence at least 90% identical to SEQ ID NO: 11.

13. The composition of any one of claims 9-12, wherein the HBV genome is of one or more of genotypes A, B, C, D, E, F, G, H, I and J.

14. The composition of any one of claims 9-13, wherein the lipid nanoparticles encapsulating the combination of mRNA molecules comprise a cationic lipid and at least one other lipid selected from the group consisting of anionic lipids, zwitterionic lipids, neutral lipids, steroids, polymer conjugated lipids, phospholipids, glycolipids, and combinations thereof.

15. A combination of:
(1) a first nucleic acid, preferably a first mRNA molecule, comprising a polynucleotide sequence encoding a first transcription activator like effector nuclease (TALEN) monomer comprising a first TALE DNA binding domain and a first FokI nuclease catalytic domain, wherein the first TALE DNA binding domain is capable of binding to a first half-site sequence of a target nucleic acid sequence within an HBV genome, and the first half-site sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 1; and
(2) a second nucleic acid, preferably a second mRNA molecule, comprising a polynucleotide sequence encoding a second transcription activator like effector nuclease (TALEN) monomer comprising a second TALE DNA binding domain and a second FokI nuclease catalytic domain, wherein the second TALE DNA binding domain is capable of binding to a second half-site sequence of the target nucleic acid sequence, and the second half-site sequence comprises a polynucleotide sequence at least about 90% identical, such as at least 90%, 91%, 92%, 93%, 94%, 95%, 96%, 97%, 98%, 99% or 100% identical, to the nucleic acid sequence of SEQ ID NO: 2;
wherein the first TALEN monomer and the second TALEN monomer are capable of forming a dimer that cleaves the target nucleic acid sequence when the first TALE DNA binding domain binds to the first half-site sequence and the second TALE DNA binding domain binds to the second half-site sequence.

16. The combination of claim 15, wherein the HBV genome is of one or more of genotypes A, B, C, D, E, F, G, H, I and J.

17. A composition comprising the combination of claim 15 or 16, wherein the first and second nucleic acids are separately or jointly encapsulated in lipid nanoparticles.

18. A nucleic acid molecule encoding at least one of the first mRNA molecule and the second mRNA molecule of claim 15.

19. An isolated host cell comprising the nucleic acid molecule of claim 18.

20. The isolated host cell of claim 19, wherein the host cell is a hepatocyte.

21. A pharmaceutical composition comprising the composition of any one of claims 9-14 or 17, the combination of claim 15 or 16, the nucleic acid molecule of claim 18, or the isolated host cell of claim 19 or 20, and a pharmaceutically acceptable carrier.

22. A method of cleaving a target nucleic acid sequence in an HBV genome, comprising contacting the HBV genome with the composition of any one of claims 9-14 or 17, the combination of claim 15 or 16, the nucleic acid molecule of claim 18, the isolated host cell of claim 19 or 20, or the pharmaceutical composition of claim 21.

23. A method for inducing gene editing of a target nucleic acid sequence in an HBV genome, comprising contacting the HBV genome with the composition of any one of claims 9-14 or 17, the combination of claim 15 or 16, the nucleic acid molecule of claim 18, the isolated host cell of claim 19 or 20, or the pharmaceutical composition of claim 21.

24. The method of claim 22 or 23, wherein the HBV genome is of one or more of genotypes A, B, C, D, E, F, G, H, I and J.

25. A method for treating a hepatitis infection in a subject in need thereof, comprising administering to the subject the pharmaceutical composition according to claim 21.

26. A method for reducing infection and/or replication of HBV in a subject, comprising administering to the subject the pharmaceutical composition according to claim 21.

27. The method of claim 25 or 26, further comprising administering to the subject a second therapeutic composition, preferably comprising an anti-HBV agent.

28. The method of any one of claims 25-27, wherein the subject has an HBV infection, preferably a chronic HBV infection.

29. The method of any one of claims 25-28, wherein the subject is co-infected with HBV and HDV.

30. The method of any one of claims 25-29, wherein an expression level of one or more of HBsAg, HBeAg, HBV DNA, HBV cccDNA, or integrated HBV DNA is reduced in the subject.

31. The method of claim 30, wherein the expression level is a hepatocyte level, a nuclear or cellular level, a liver level, a serum level, or a plasma level.

32. A method of producing a TALEN comprising transcribing the nucleic acid molecule of claim 18, in vitro or in vivo.

33. The pharmaceutical composition of claim 21 for use in treating a hepatitis B virus (HBV)-induced disease in a subject in need thereof, preferably wherein the subject has chronic HBV infection.

34. The pharmaceutical composition of claim 33, wherein the HBV-induced disease is selected from the group consisting of advanced fibrosis, cirrhosis and hepatocellular carcinoma (HCC), optionally in combination with another therapeutic agent, preferably another anti-HBV agent.
